SEQUENCE LISTING (41 sequences)

SEQ ID NO: 1 - DNA, 21 bp, Artificial Sequence
  Synthesized oligonucleotide and RNAi agent
cuuacgcugaguacuucgatt

SEQ ID NO: 2 - DNA, 21 bp, Artificial Sequence
  Synthesized oligonucleotide and RNAi agent
ucgaaguacucagcguaagtt

SEQ ID NO: 3 - DNA, 21 bp, Artificial Sequence
  Synthesized oligonucleotide and RNAi agent
cuuacgcugaguacuucgatt

SEQ ID NO: 4 - DNA, 21 bp, Artificial Sequence
  Synthesized oligonucleotide and RNAi agent
ucgaaguacucagcguaagtt

SEQ ID NO: 5 - DNA, 21 bp, Artificial Sequence
  Synthesized oligonucleotide and RNAi agent
cuuacgcugaguacuucgatt

SEQ ID NO: 6 - DNA, 21 bp, Artificial Sequence
  Synthesized oligonucleotide and RNAi agent
ucgaaguacucagcguaagtt

SEQ ID NO: 7 - DNA, 21 bp, Artificial Sequence
  Synthesized oligonucleotide and RNAi agent
cuuacgcugaguacuucgatt

SEQ ID NO: 8 - DNA, 21 bp, Artificial Sequence
  Synthesized oligonucleotide and RNAi agent
ucgaaguacucagcguaagtt

SEQ ID NO: 9 - DNA, 21 bp, Artificial Sequence
  Synthesized oligonucleotide and RNAi agent
ggaucaucucaagucuuactt

SEQ ID NO: 10 - DNA, 21 bp, Artificial Sequence
  Synthesized oligonucleotide and RNAi agent
guaagacuugagaugaucctt

SEQ ID NO: 11 - DNA, 21 bp, Artificial Sequence
  Synthesized oligonucleotide and RNAi agent
ggaucaucucaagucuuactt

SEQ ID NO: 12 - DNA, 21 bp, Artificial Sequence
  Synthesized oligonucleotide and RNAi agent
guaagacuugagaugaucctt

SEQ ID NO: 13 - protein, 29 aa, Artificial Sequence
  Exemplary endosomolytic peptide
AALEALAEALEALAEALEALAEAAAAGGC

SEQ ID NO: 14 - protein, 30 aa, Artificial Sequence
  Exemplary endosomolytic peptide
AALAEALAEALAEALAEALAEALAAAAGGC

SEQ ID NO: 15 - protein, 15 aa, Artificial Sequence
  Exemplary endosomolytic peptide
ALEALAEALEALAEA

SEQ ID NO: 16 - protein, 22 aa, Artificial Sequence
  Exemplary endosomolytic peptide
GLFEAIEGFIENGWEGMIWDYG

SEQ ID NO: 17 - protein, 23 aa, Artificial Sequence
  Exemplary endosomolytic peptide
GLFGAIAGFIENGWEGMIDGWYG

SEQ ID NO: 18 - protein, 48 aa, Artificial Sequence
  Exemplary endosomolytic peptide
GLFEAIEGFIENGWEGMIDGWYGCGLFEAIEGFIENGWEGMIDGWYGC

SEQ ID NO: 19 - protein, 44 aa, Artificial Sequence
  Exemplary endosomolytic peptide
GLFEAIEGFIENGWEGMIDGGCGLFEAIEGFIENGWEGMIDGGC

SEQ ID NO: 20 - protein, 35 aa, Artificial Sequence
  Exemplary endosomolytic peptide
GLFGALAEALAEALAEHLAEALAEALEALAAGGSC

SEQ ID NO: 21 - protein, 34 aa, Artificial Sequence
  Exemplary endosomolytic peptide
GLFEAIEGFIENGWEGLAEALAEALEALAAGGSC

SEQ ID NO: 22 - protein, 41 aa, Artificial Sequence
  Exemplary endosomolytic peptide
GLFEAIEGFIENGWEGLIDGKGLFEAIEGFIENGWEGLIDG

SEQ ID NO: 23 - protein, 16 aa, Artificial Sequence
  Exemplary cell permeation peptide
RQIKIWFQNRRMKWKK

SEQ ID NO: 24 - protein, 14 aa, Artificial Sequence
  Exemplary cell permeation peptide
GRKKRRQRRRPPQC

SEQ ID NO: 25 - protein, 27 aa, Artificial Sequence
  Exemplary cell permeation peptide
GALFLGWLGAAGSTMGAWSQPKKKRKV

SEQ ID NO: 26 - protein, 18 aa, Artificial Sequence
  Exemplary cell permeation peptide
LLIILRRRIRKQAHAHSK

SEQ ID NO: 27 - protein, 26 aa, Artificial Sequence
  Exemplary cell permeation peptide
GWTLNSAGYLLKINLKALAALAKKIL

SEQ ID NO: 28 - protein, 18 aa, Artificial Sequence
  Exemplary cell permeation peptide
KLALKLALKALKAALKLA

SEQ ID NO: 29 - protein, 9 aa, Artificial Sequence
  Exemplary cell permeation peptide
RRRRRRRRR

SEQ ID NO: 30 - protein, 10 aa, Artificial Sequence
  Exemplary cell permeation peptide
KFFKFFKFFK

SEQ ID NO: 31 - protein, 37 aa, Artificial Sequence
  Exemplary cell permeation peptide
LLGDFFRKSKEKIGKEFKRIVQRIKDFLRNLVPRTES

SEQ ID NO: 32 - protein, 31 aa, Artificial Sequence
  Exemplary cell permeation peptide
SWLSKTAKKLENSAKKRISEGIAIAIQGGPR

SEQ ID NO: 33 - protein, 30 aa, Artificial Sequence
  Exemplary cell permeation peptide
ACYCRIPACIAGERRYGTCIYQGRLWAFCC

SEQ ID NO: 34 - protein, 36 aa, Artificial Sequence
  Exemplary cell permeation peptide
DHYNCVSSGGQCLYSACPIFTKIQGTCYRGKAKCCK

SEQ ID NO: 35 - protein, 12 aa, Artificial Sequence
  Exemplary cell permeation peptide
RKCRIVVIRVCR

SEQ ID NO: 36 - protein, 42 aa, Artificial Sequence
  Exemplary cell permeation peptide
RRRPRPPYLPRPRPPPFFPPRLPPRIPPGFPPRFPPRFPGKR

SEQ ID NO: 37 - protein, 13 aa, Artificial Sequence
  Exemplary cell permeation peptide
ILPWKWPWWPWRR

SEQ ID NO: 38 - protein, 16 aa, Artificial Sequence
  Exemplary hydrophobic MTS-containing peptide, RFGF
AAVALLPAVLLALLAP

SEQ ID NO: 39 - protein, 11 aa, Artificial Sequence
  Exemplary hydrophobic MTS-containing peptide, RFGF analogue
AALLPVLLAAP

SEQ ID NO: 40 - protein, 13 aa, Artificial Sequence
  Exemplary cell membrane delivery peptide, sequences from the HIV Tat protein
GRKKRRQRRRPPQ

SEQ ID NO: 41 - protein, 16 aa, Artificial Sequence
  Exemplary cell membrane delivery peptide, the Drosophila Antennapedia protein
RQIKIWFQNRRMKWKK

(19) United States
(12) Patent Application Publication
(10) Pub. No.: US 2011/0130440 A1
(43) Pub. Date: Jun. 2, 2011

(54) NON-NATURAL RIBONUCLEOTIDES, AND METHODS OF USE THEREOF

(75) Inventors: Muthiah Manoharan, Cambridge, MA (US); Kallanthottathil G. Rajeev, Cambridge, MA (US)

(73) Assignee: ALNYLAM PHARMACEUTICALS, INC., Cambridge, MA (US)

(21) Appl. No.: 12/934,323

(22) PCT Filed: Mar. 26, 2009
(86) PCT No.: PCT/US09/38425

§ 371 Date: Dec. 29, 2010

Related U.S. Application Data
(60) Provisional application No. 61/039,574, filed on Mar. 26, 2008.
(60) Provisional application No. 61/040,414, filed on Mar. 28, 2008.
(60) Provisional application No. 61/105,307, filed on Oct. 14, 2008.

(51) Int. Cl. A61K 31/7105; C07H 19/23; C07H 7/06; C07H 19/06; C07H 21/00; C07H 21/02; C12N 5/071

(52) U.S. Cl. 514/44 A; 536/27.1; 536/26.1; 536/29.2; 536/28.54; 536/27.13; 536/26.7; 536/23.1; 536/24.5; 435/375

(57) ABSTRACT

One aspect of the present invention relates to modified nucleosides and oligonucleotides comprising such modified nucleosides. Another aspect of the invention relates to a method of inhibiting the expression of a gene in call, the method comprising (a) contacting an oligonucleotide of the invention with the cell; and (b) maintaining the cell from step (a) for a time sufficient to obtain degradation of the mRNA of the target gene.

### CROSS-REFERENCE TO RELATED APPLICATIONS

This application claims priority to PCT Application No. PCT/US09/38425, filed Mar. 26, 2009, which claims the benefit of priority to U.S. Provisional Patent Application No. 61/039,574, filed Mar. 26, 2008; U.S. Provisional Patent Application No. 61/040,414, filed Mar. 28, 2008; U.S. Provisional Patent Application No. 61/105,307, filed Oct. 14, 2008, all of which are hereby incorporated by reference in their entirety.

### TECHNICAL FIELD

The invention relates to modified oligonucleotide such as modified RNA interference agents.

### BACKGROUND

Oligonucleotides and their analogs have been developed for various uses in molecular biology, including use as probes, primers, linkers, adapters, and gene fragments. In a number of these applications, the oligonucleotides specifically hybridize to a target nucleic acid sequence. Hybridization is the sequence specific hydrogen bonding of oligonucleotides via Watson-Crick and/or Hoogsteen base pairs to RNA or DNA. The bases of such base pairs are said to be complementary to one another.

Double-stranded RNA molecules (dsRNA) can block gene expression by virtue of a highly conserved regulatory mechanism known as RNA interference (RNAi). Briefly, the RNA III Dicer enzyme processes dsRNA into small interfering RNA (also sometimes called short interfering RNA or siRNA) of approximately 22 nucleotides. One strand of the siRNA (the “antisense strand”) then serves as a guide sequence to induce cleavage of messenger RNAs (mRNAs) including a nucleotide sequence which is at least partially complementary to the sequence of the antisense strand by an RNA-induced silencing complex, RISC. The antisense strand is not cleaved or otherwise degraded in this process, and the RISC including the antisense strand can subsequently affect the cleavage of further mRNAs.

It is greatly desired that oligonucleotides be able to be synthesized to have customized properties which are tailored for desired uses. Thus a number of chemical modifications have been introduced into oligonucleotides to increase their usefulness in diagnostics, as research reagents and as therapeutic entities. (For example, pseudouridine derivatives and lipid-containing oligonucleotides, as discussed below.) Such modifications include those designed to increase binding to a target strand (i.e., increase their melting temperatures, Tₘ), to assist in identification of the oligonucleotide or an oligonucleotide-target complex, to increase cell penetration, to stabilize against nucleases and other enzymes that degrade or interfere with the structure or activity of the oligonucleotides, to provide a mode of disruption (a terminating event) once sequence-specifically bound to a target, and to improve the pharmacokinetic properties of the oligonucleotide.

Even given the advances which have already been made in the art, there remains a need for new modifications designed to, for example, increase binding to a target strand (i.e., increase their melting temperatures, Tₘ), to assist in identification of the oligonucleotide or an oligonucleotide-target complex, to increase cell penetration, to stabilize against nucleases and other enzymes that degrade or interfere with the structure or activity of the oligonucleotides, to provide a mode of disruption (a terminating event) once sequence-specifically bound to a target, and to improve the pharmacokinetic properties of the oligonucleotide.

### SUMMARY

In one aspect, the present invention provides an oligonucleotide comprising at least one modified nucleoside selected from the group consisting of

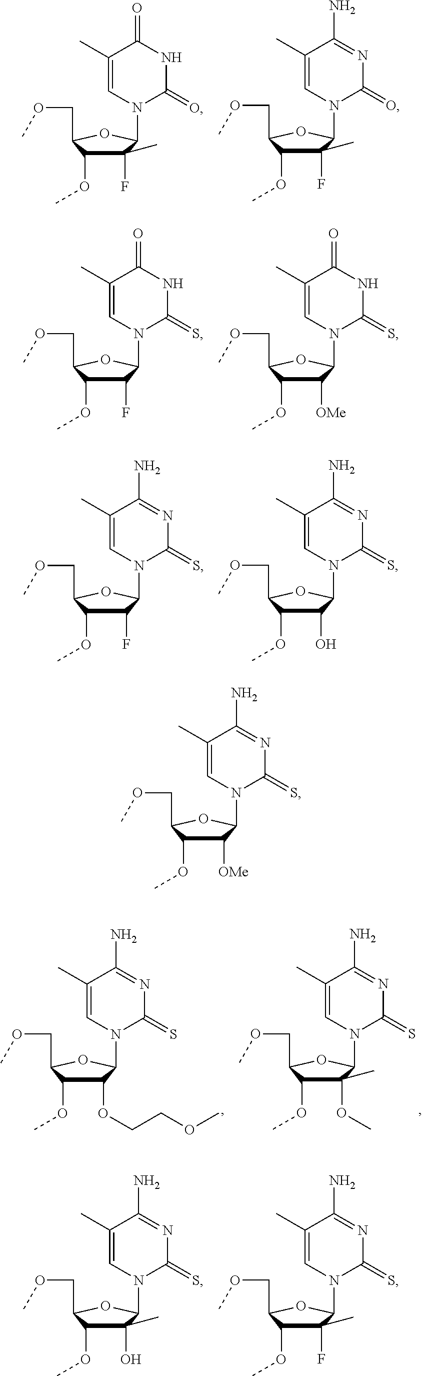

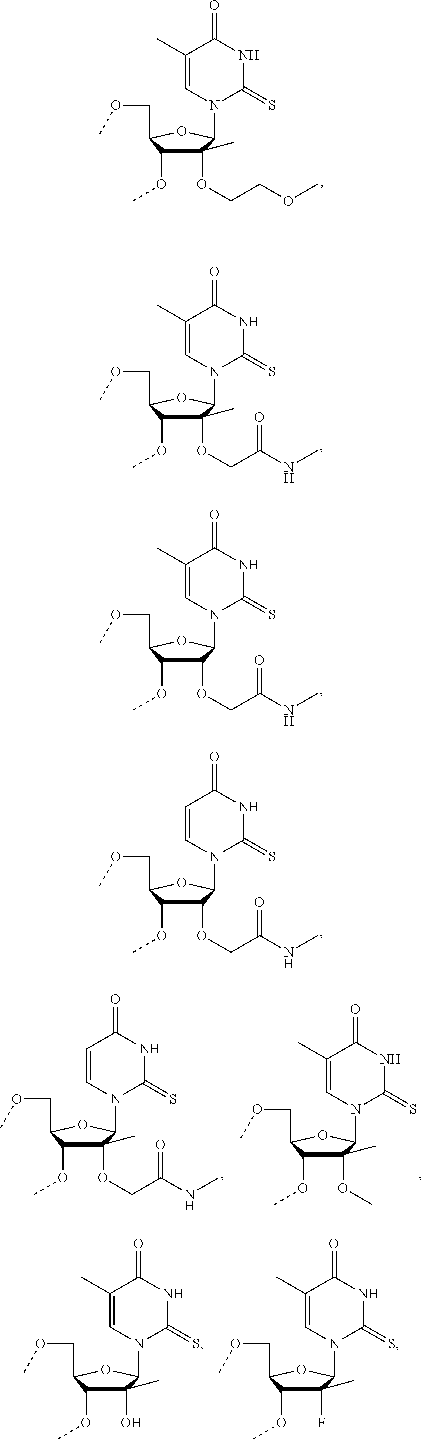

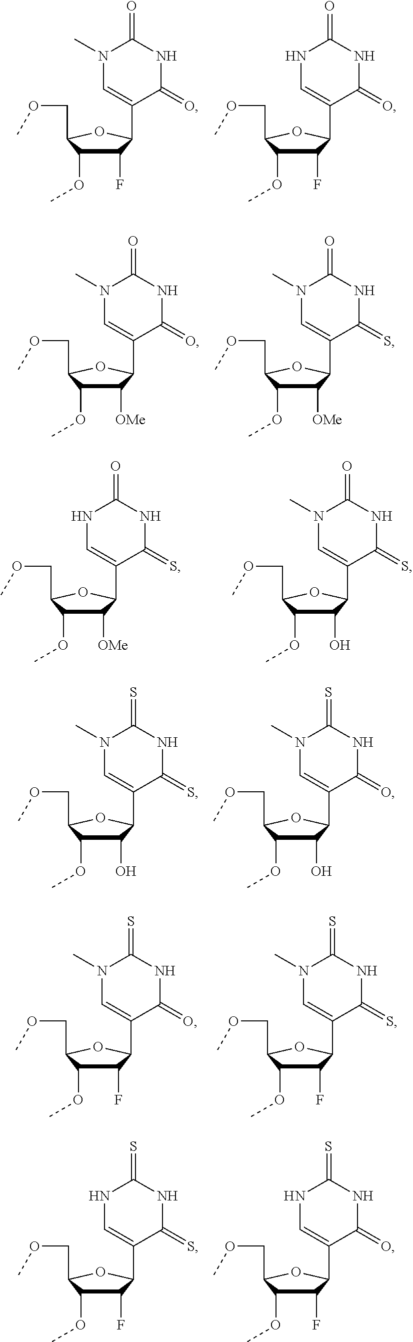

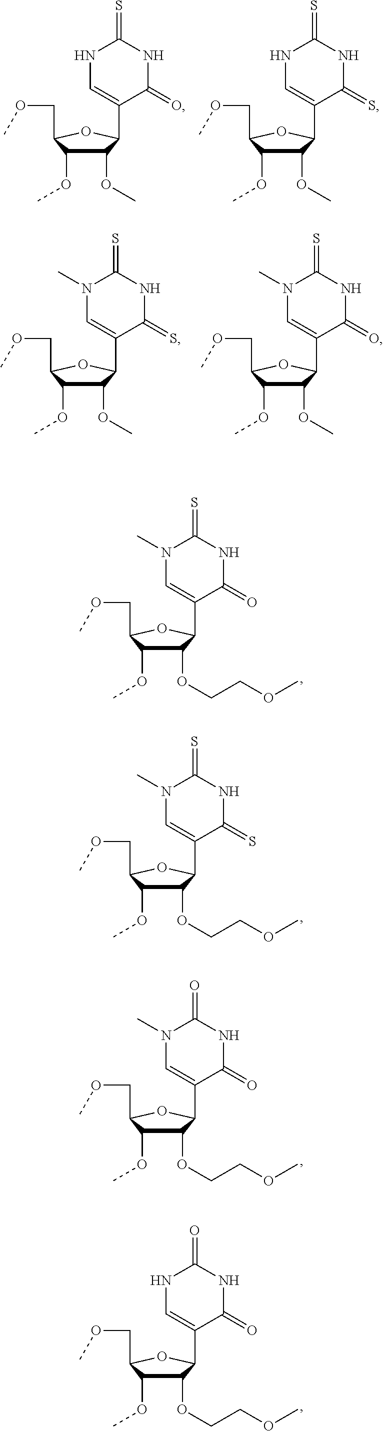

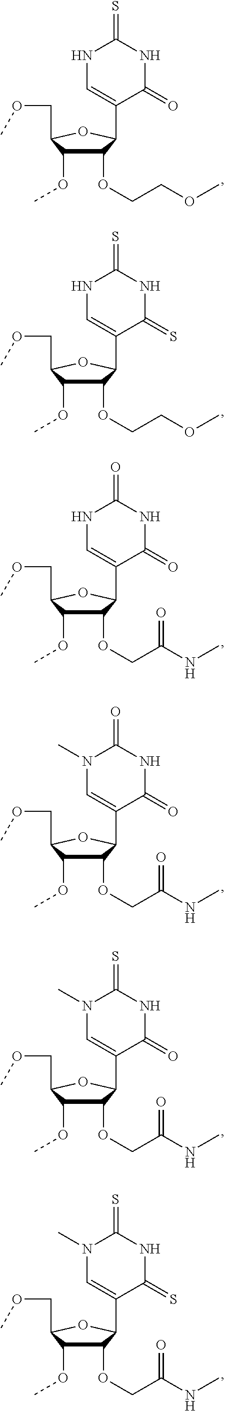

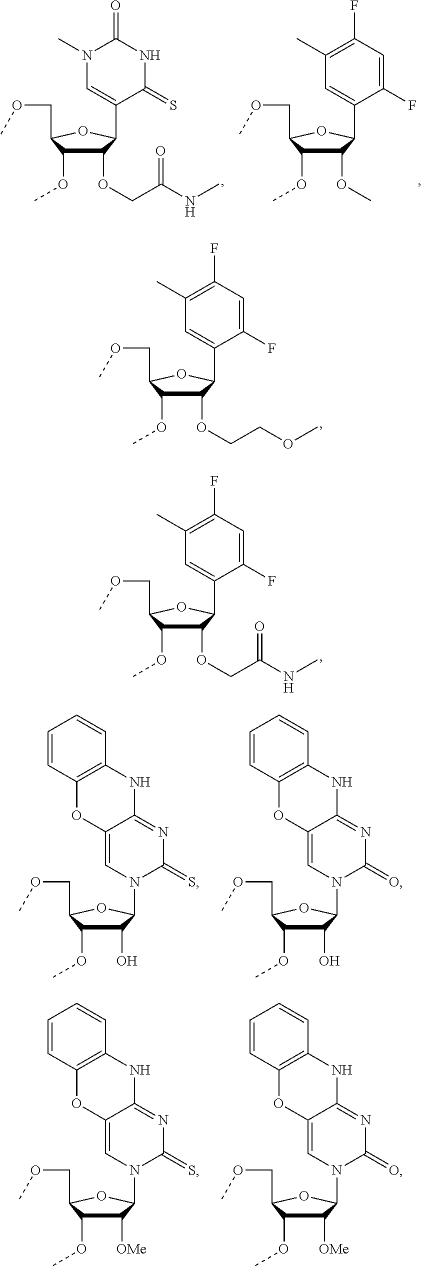

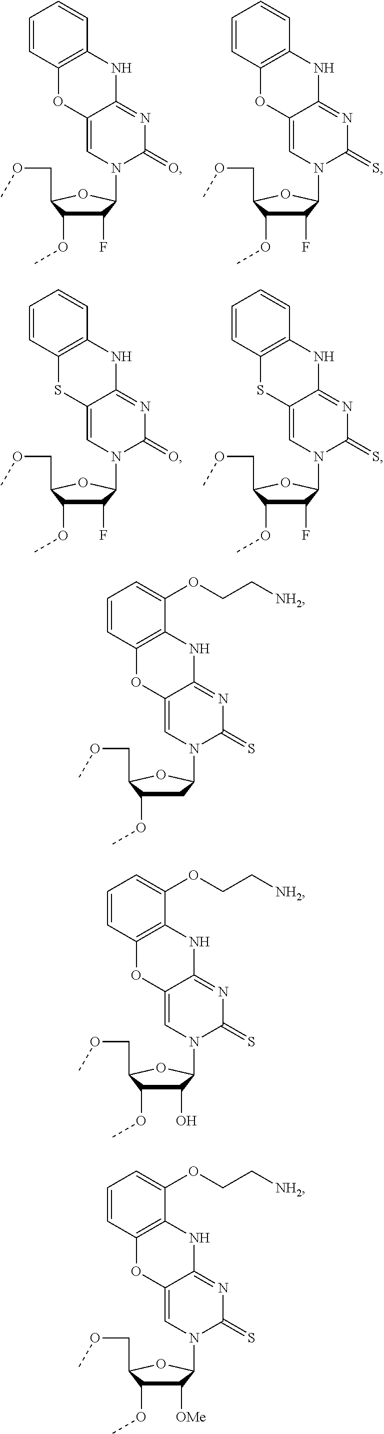

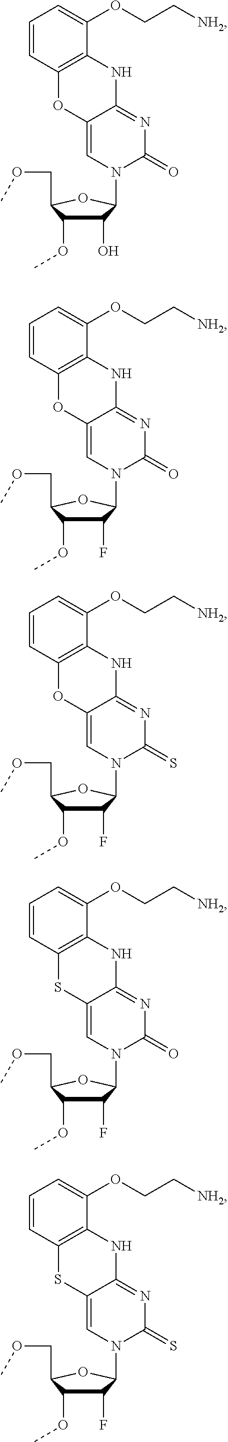

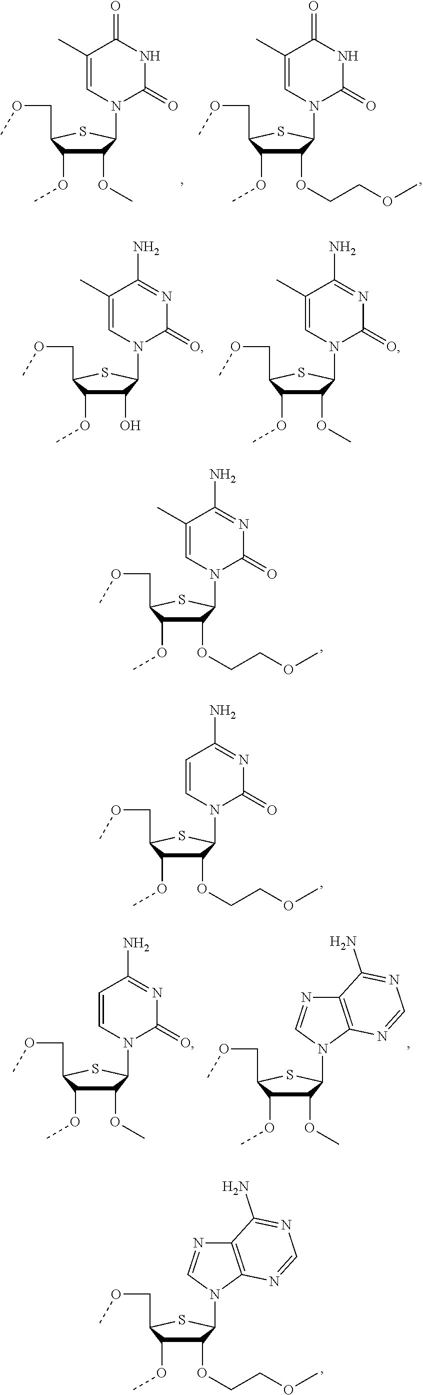

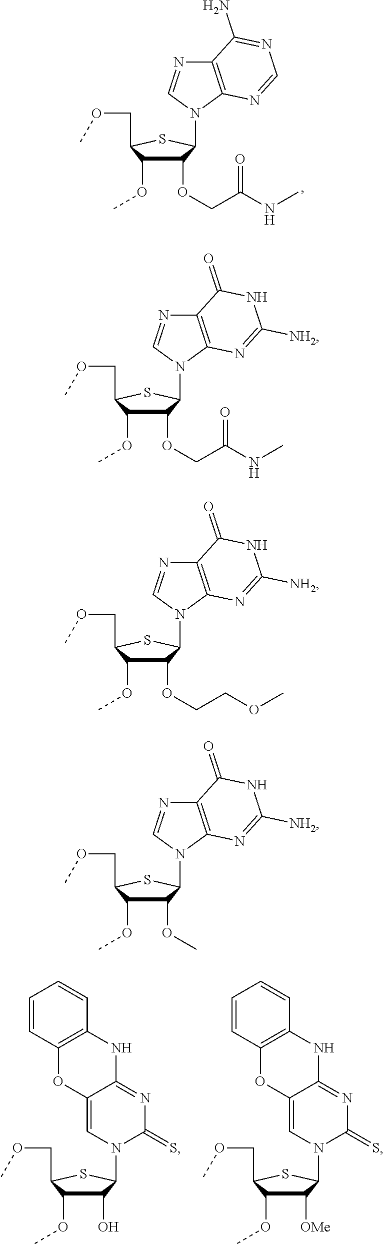

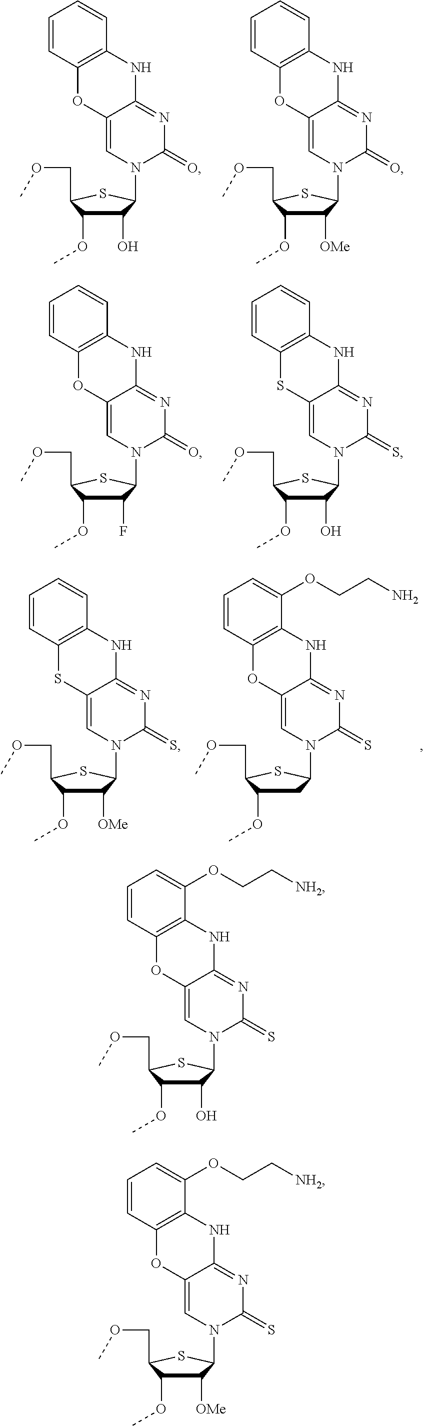

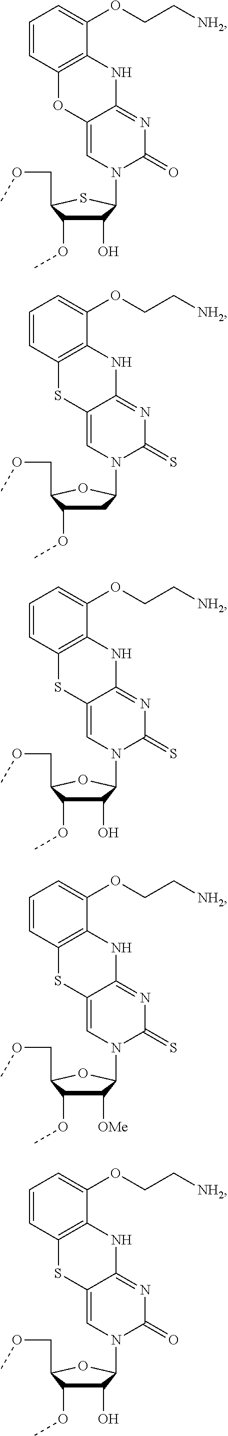

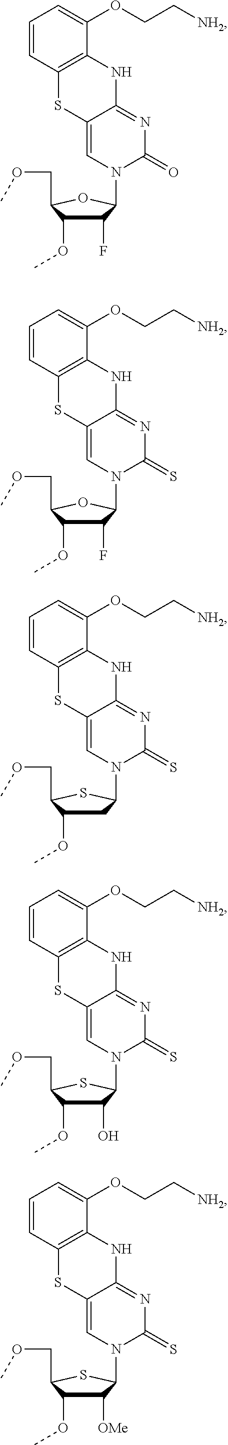

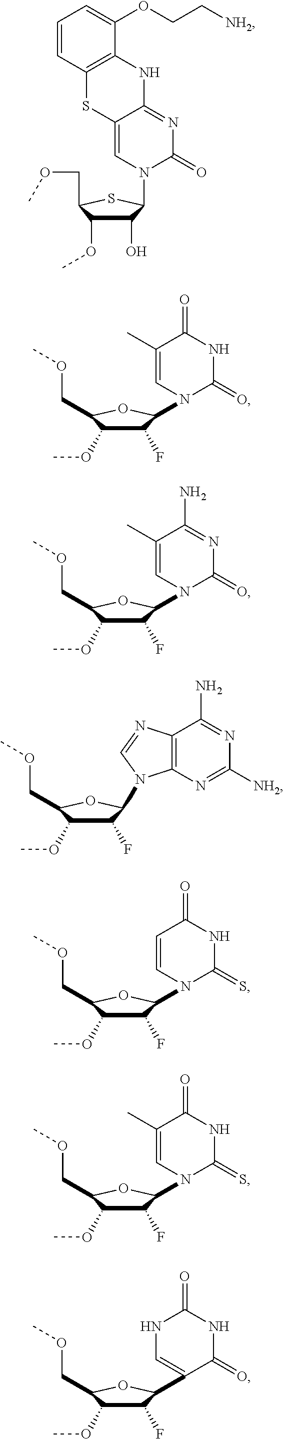

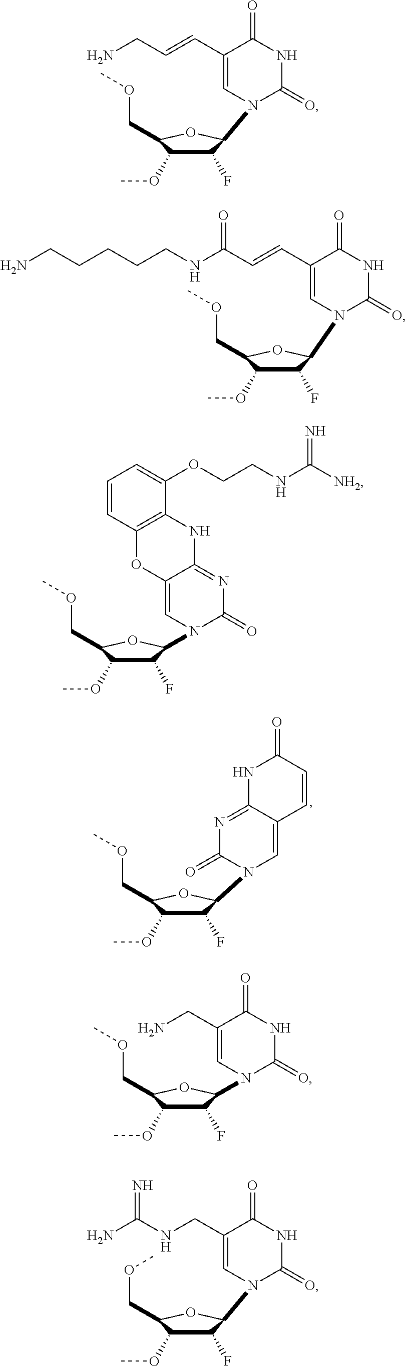

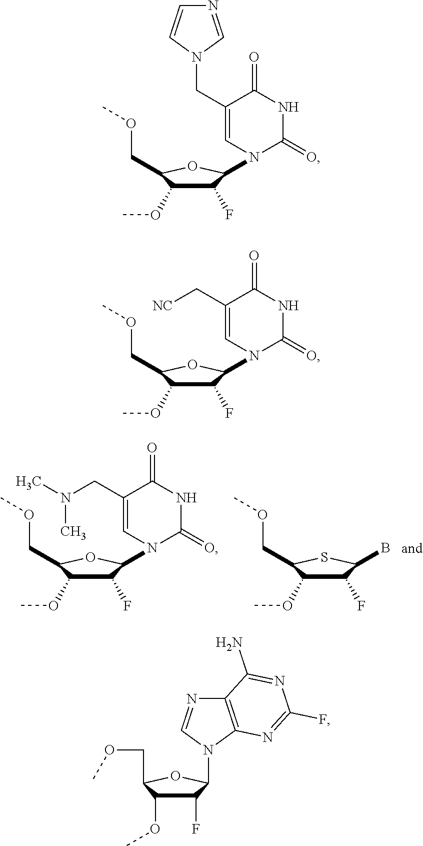

wherein B is an optionally substituted nucleobase and—is H, protecting group, nucleotide, oligonucleotide, ligand or a ligand carrying monomer, provided that—is nucleotide or oligonucleotide at least once.

Another aspect of the invention relates to a method of inhibiting the expression of a gene in call, the method comprising (a) contacting an oligonucleotide of the invention with the cell; and (b) maintaining the cell from step (a) for a time sufficient to obtain degradation of the mRNA of the target gene.

### BRIEF DESCRIPTION OF THE FIGURES

FIGS. 1-10 depict high affinity modified nucleosides.

FIGS. 11 and 12 depict prophetic reaction schemes for the preparation of some of the compounds of the present invention.

FIG. 13 depicts a synthesis of a 2′,2-difluoro-2′-deoxyadanosine.

FIGS. 14 and 15 depict a synthesis of phosphoramidite and controlled pore glass support of N¹-(N-methyl-3-propionamide)-pseudouridine.

FIGS. 16 and 17 depict a synthesis of controlled pore glass support of N¹-(cholesteryl-alkyl)-pseudouridine.

FIG. 18 depicts a synthesis of N¹- and N¹,N³-(2-cyanoethyl)-pseudouridines (FIG. 18A); and a synthesis of N¹-(3-phthalimido-porpyl)-pseudouridines (FIG. 18B).

FIG. 19 depicts the preparation of phosphoramidite and controlled pore glass support of substituted-pseudouridines.

FIG. 20 depicts the preparation of phosphoramidite and controlled pore glass support of 2′-substituted N¹-alkylated pseudouridines.

FIG. 21 depicts the preparation of phosphoramidite and controlled pore glass support of 2′-deoxy-2′-fluoro-4′-thio-5-methyl uracil and cytosine.

FIG. 22 depicts routes to the preparation of phosphoramidite and controlled pore glass support of selected modified nucleosides.

FIG. 23 depicts routes to the preparation of phosphoramidite and controlled pore glass support of selected modified nucleosides.

FIG. 24 depicts routes to the preparation of phosphoramidite and controlled pore glass support of selected nucleosides and oligonucleotides.

FIG. 25 depicts routes to the preparation of phosphoramidite and controlled pore glass support of 5′-O-(4,4′-dimethoxitrityl)-2′-O-(tert-butyldimethylsilyl)-1-N-methylpseudo-uridine.

FIGS. 26A and 26B show photographs of results of the serum stability assays

FIG. 27 depicts results of the dual luciferase gene silencing assays.

### DETAILED DESCRIPTION

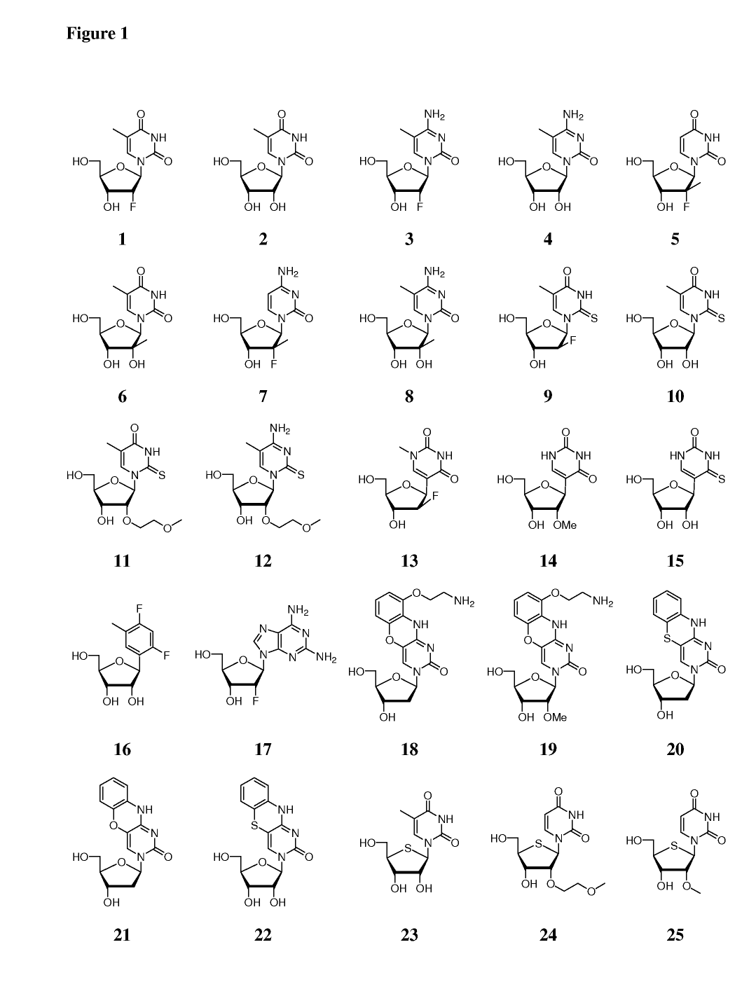

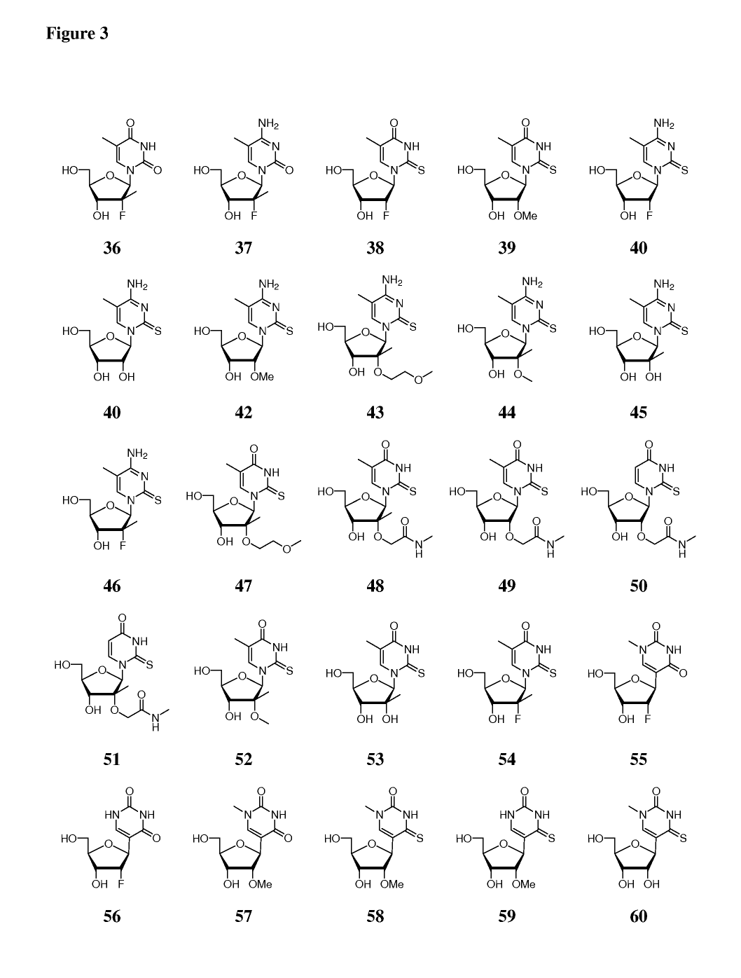

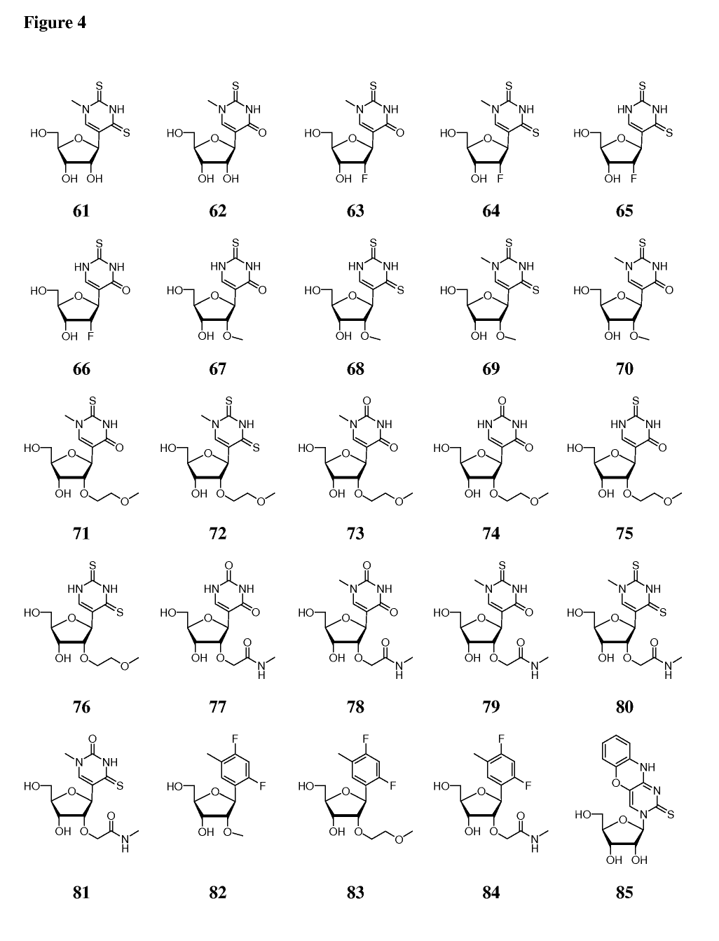

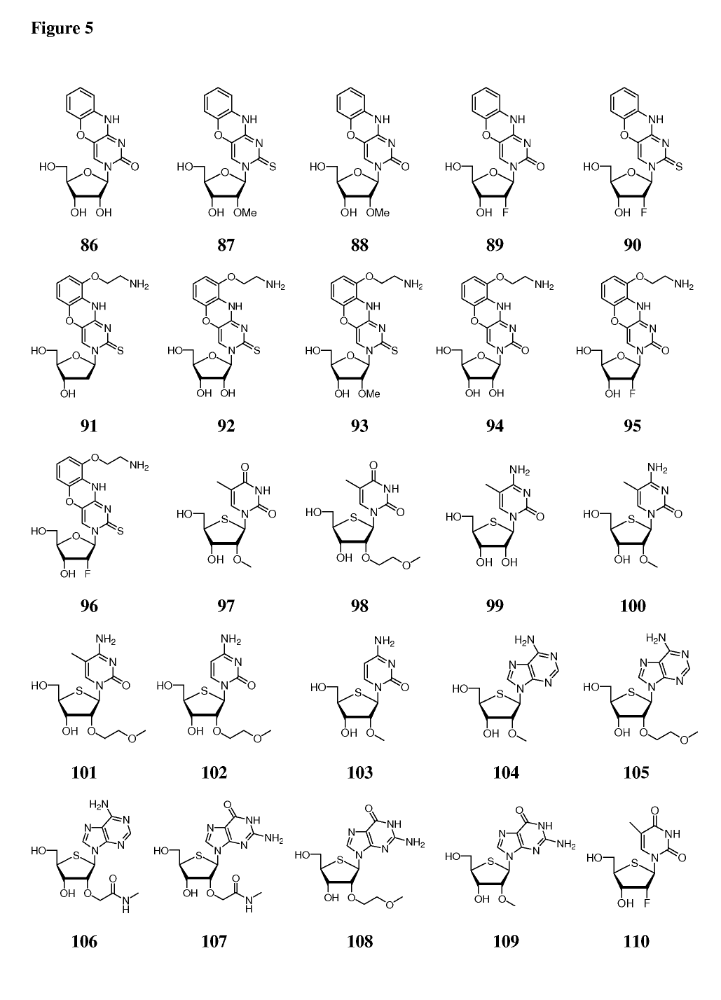

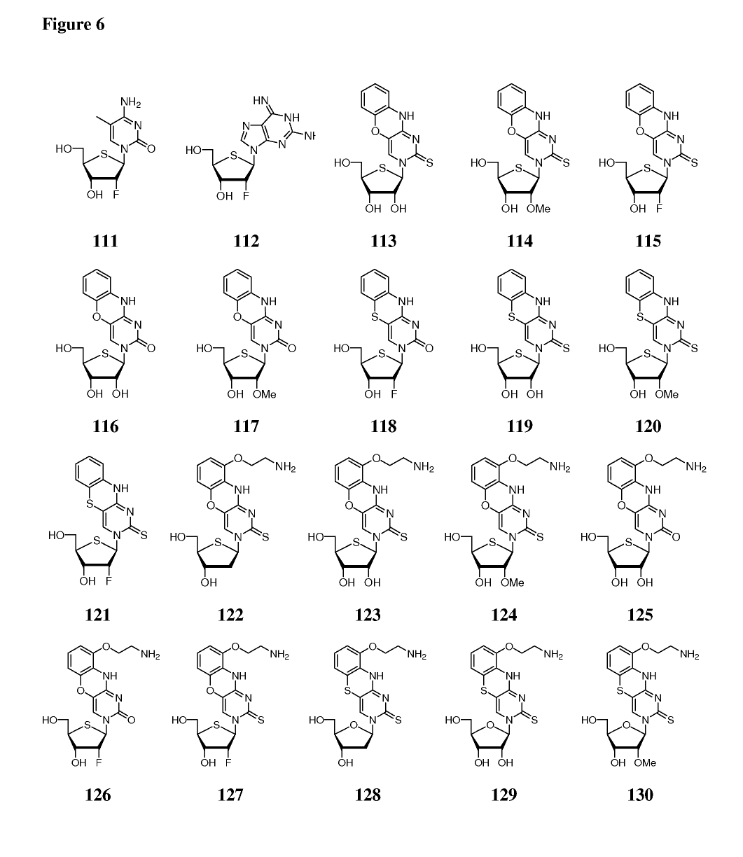

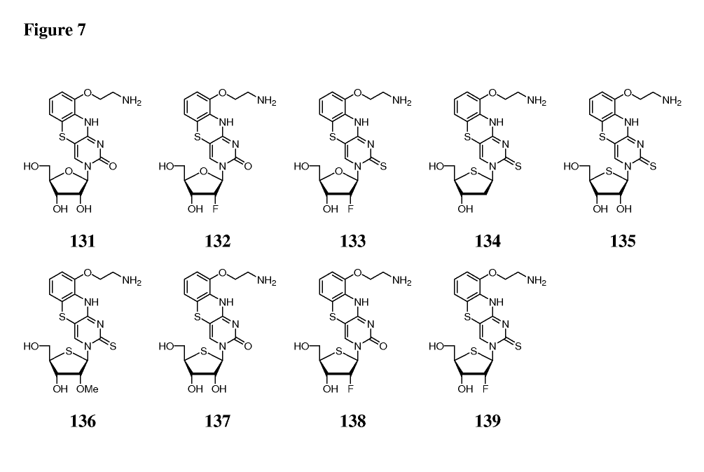

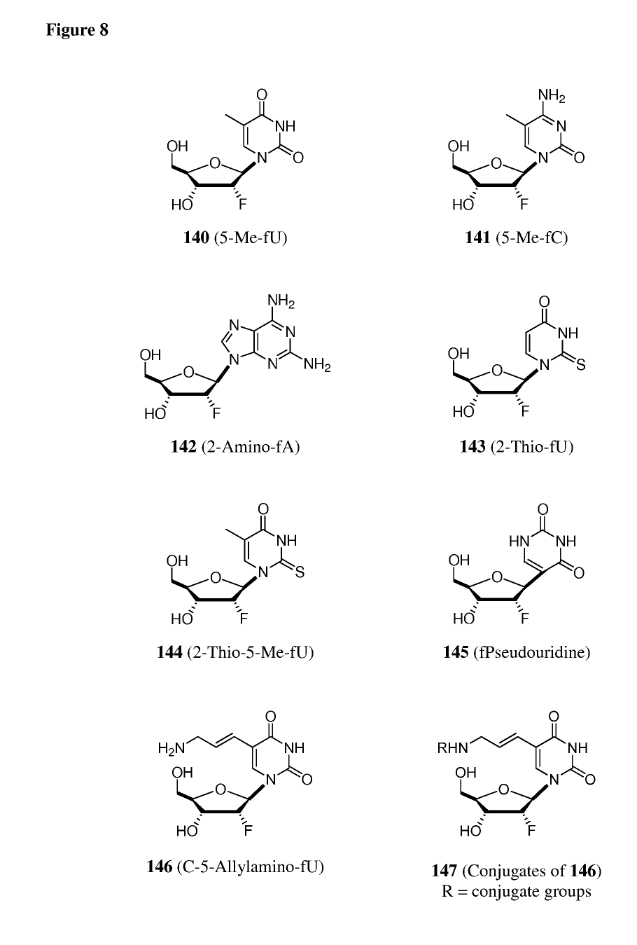

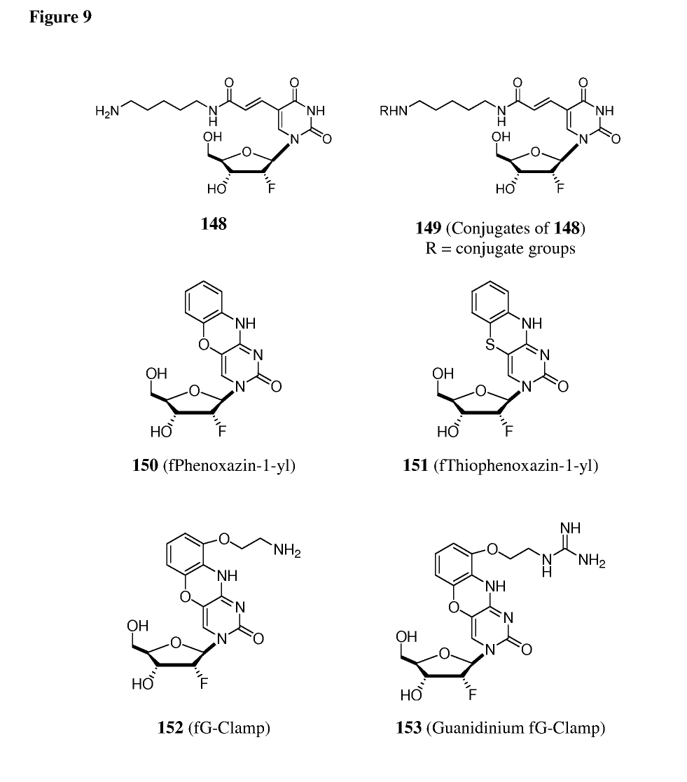

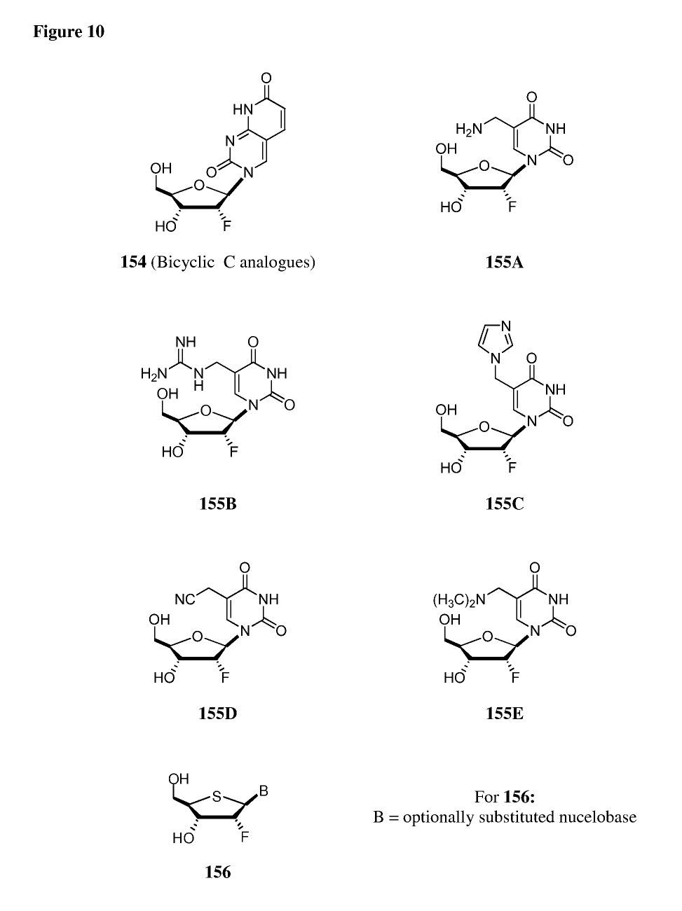

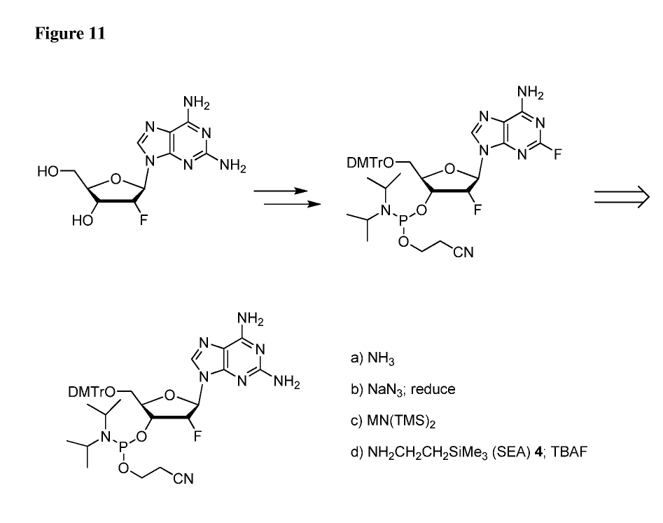

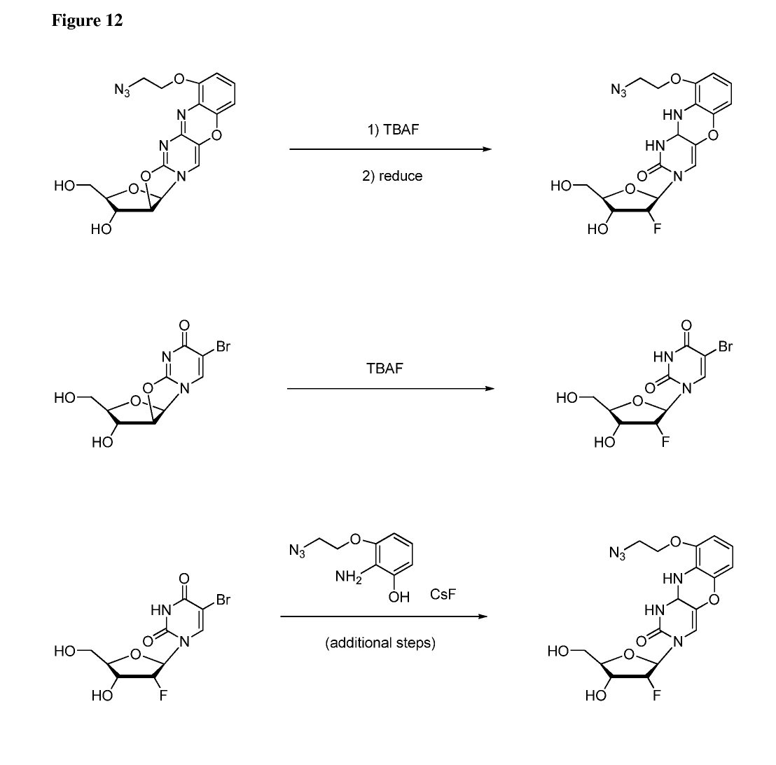

In one aspect, the invention provides modified nucleosides shown in FIGS. 1-10.

In another aspect, the invention provides oligonucleotides comprising modified nucleosides, as well as methods for inhibiting the expression of a target gene in a cell, tissue or a mammal using these oligonucleotides.

In one embodiment, the modified nucleoside is chosen from a group consisting of

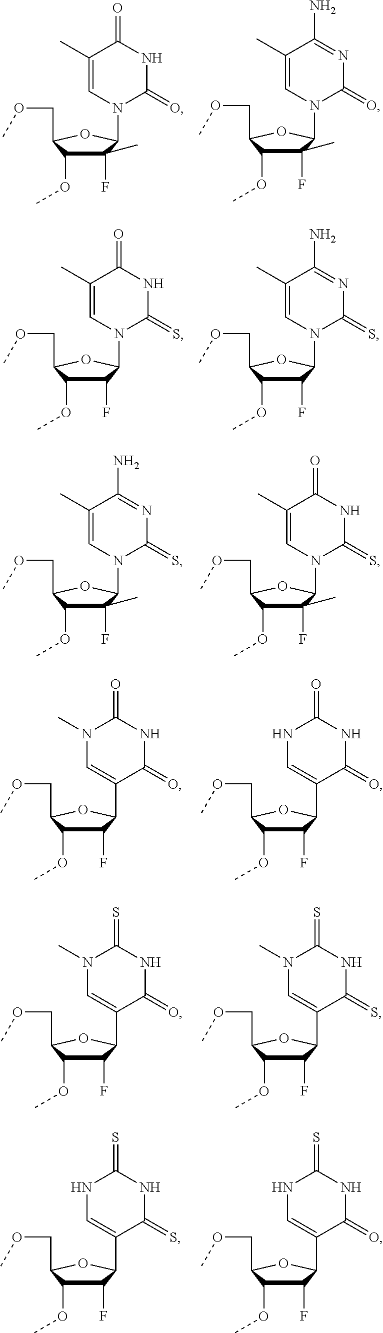

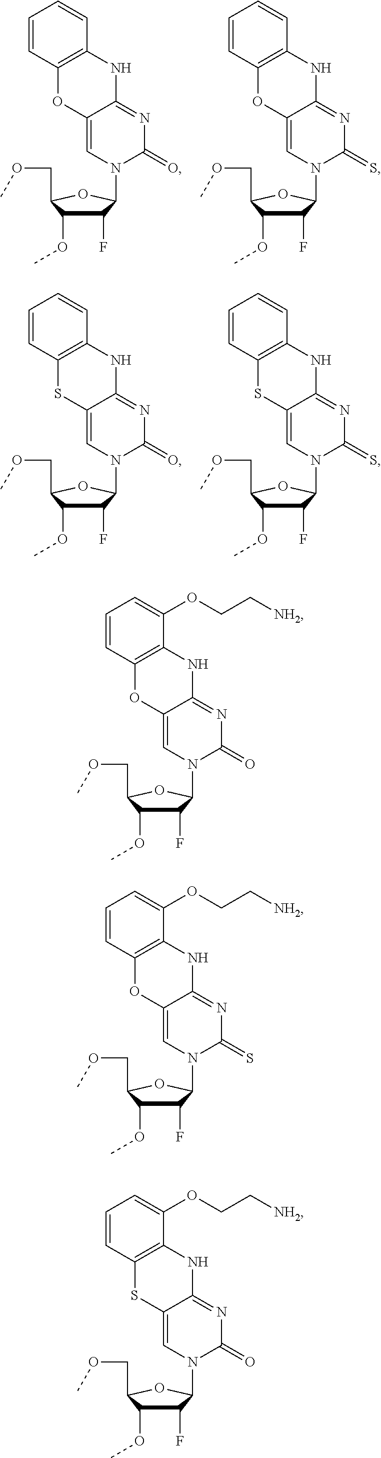

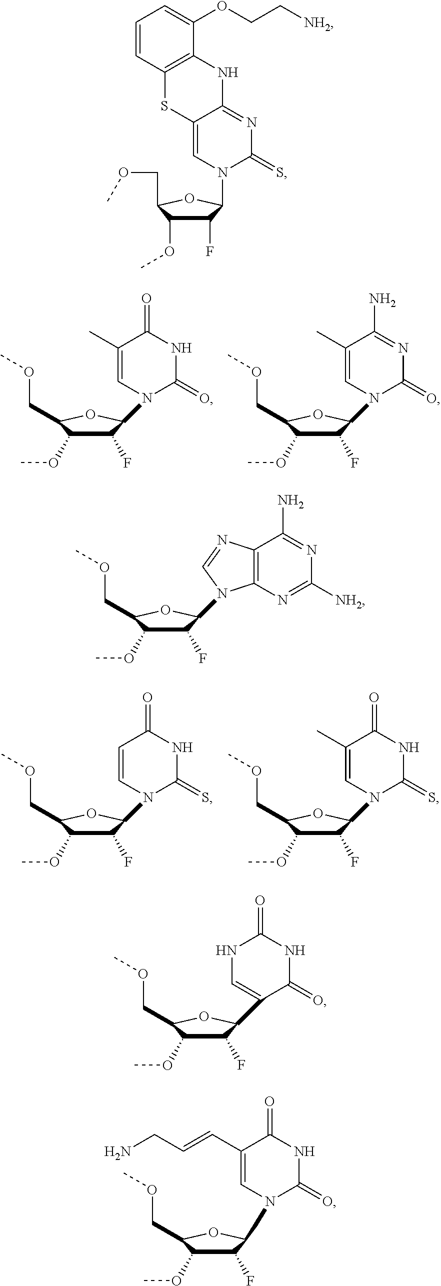

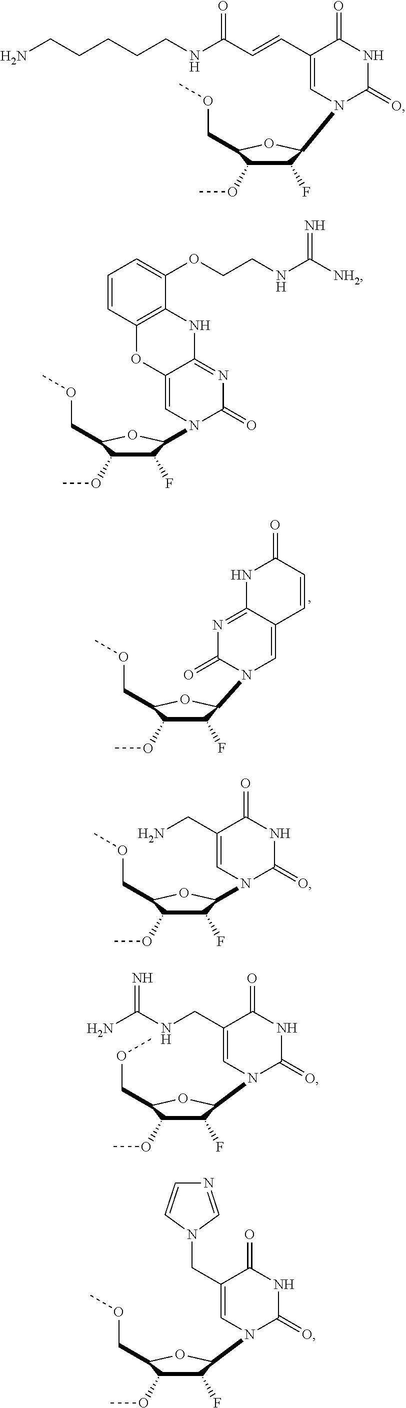

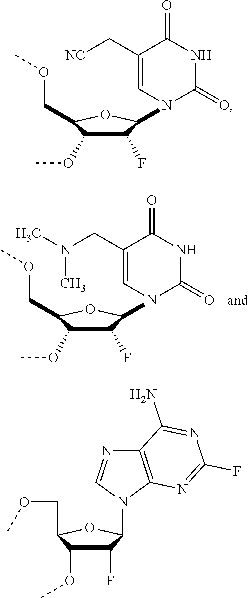

wherein—is H, protecting group, nucleotide, oligonucleotide, ligand or a ligand carrying monomer, provided that—is nucleotide or oligonucleotide at least once.

In one embodiment, the modified nucleoside is

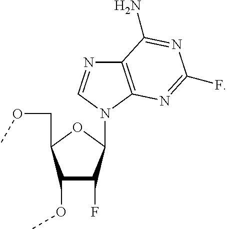

In one embodiment, the modified nucleoside is

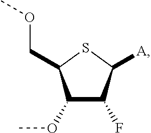

wherein A is optionally substituted nucleobase and—is H, protecting group, nucleotide, oligonucleotide, ligand or a ligand carrying monomer, provided that—is nucleotide or oligonucleotide at least once.

In one embodiment, the optionally substituted nucleobase for the modified nucleoside is chosen from a group consisting of 2-(amino)adenine, 5-(methyl)cytosine, 2-(thio)uracil,5-(methyl)-2-(thio)uracil, 4-(thio)uracil, 5-(methyl)-4-(thio)uracil, 5-(methyl)-2,4-(dithio)uracil, 5-(methyl)uracil, 5-(guanidiniumalkyl)uracil, 5-(halo)uracil, dihydrouracil, 5-uracil (i.e., pseudouracil), 2-(thio)pseudouracil,2,4-(thio)pseudouracil,2,4-(dithio)psuedouracil, 5-(methyl)pseudouracil, 5-(methyl)-2-(thio)pseudouracil, 5-(methyl)-4-(thio)pseudouracil, 5-(methyl)-2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl and 7-(guanidiniumalkylhydroxy)-1-(aza)-2-(thio)-3-(aza)-phenthiazin-1-yl.

In one embodiment, the modified nucleoside is

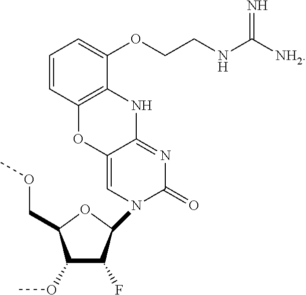

A preferred embodiment of the invention relates to a nucleoside having the formula:

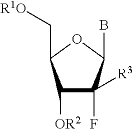

wherein:

R¹ represents —H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;

R² represents R′, —F, —(C₁-C₆)alkyl, —(C₂-C₆)allyl, —(C(R³)₂)—OR³, —(C(R³)₂)—SR³, —(C(R³)₂)ₙN(R³)₂, —(C(R³)₂)ₙC(O)N(R³)₂, —(C(R³)₂)ₙO(C₁-C₆)alkyl, —(C(R³)₂)ₙS(C₁-C₆)alkyl, —(C(R³)₂)ₙO(C(R³)₂)ₙN((C₁-C₆)alkyl)₂, —(C(R³)₂)ₙO(C₁-C₆)alkyl)₂, —C(O)R³, —C(O)R³C(O)H, —C(O)R³C(O)OH, —C(O)R³C(O)R³, —C(O)R³C(O)NR³—PO₂, —P(OR³)₂, —P(N(R³)₂)₂, —P(OR³)N(R³)₂, or a linker.

R³ represents independently for each occurrence hydrogen or a substituted or unsubstituted C₁-C₄ alkyl. Substituents for the substituted alkyl include functional groups known to those of skill in the art, including, halogens, cyano, acetyl groups. The substitutents may be attached to the backbone of the alkyl group on the terminal alkyl carbon.

M represents independently for each occurrence an alkali metal or a transition metal with an overall charge of +1; and n is an integer from 1-4.

B may represent any of the following nucleobases:

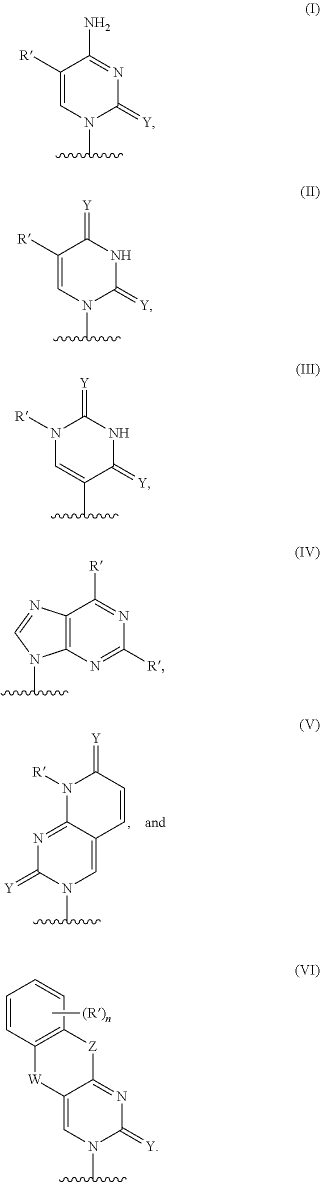

For each of formulas (I), (II), (III), (IV), (V), and (VI), each Y independently represents O or S; W and Z are each independently absent or independently represent NR″, CH₂, O or S; n is 1, 2, 3 or 4; and each R′ independently represents hydrogen, alkyl, alkenyl, alkynyl, halogen (such as F, Cl, or I), amine (including N(R″)₂, where R″ is hydrogen or alkyl), amide, carboxylic acid, ester, carbonyl (including C(O)R″, wherein R″ is hydrogen or alkyl), carboxyl, cyano, azido, aryl, heteroaryl, heterocyclic, cycloalkyl, hydroxyl, hydroxyalkyl, aminoalkyl, alkyoxy, and guanidine, wherein each of the above groups may optionally be substituted with one or more alkyl, alkenyl, alkynyl, halogen, amine, amide, carboxylic acid, ester, carbonyl, carboxyl, cyano, azido, aryl, heteroaryl, heterocyclic, cycloalkyl, hydroxyl, hydroxyalkyl, aminoalkyl, alkyoxy, or guanidine groups.

Preferably, B represents a nucleobase selected from the group consisting of:

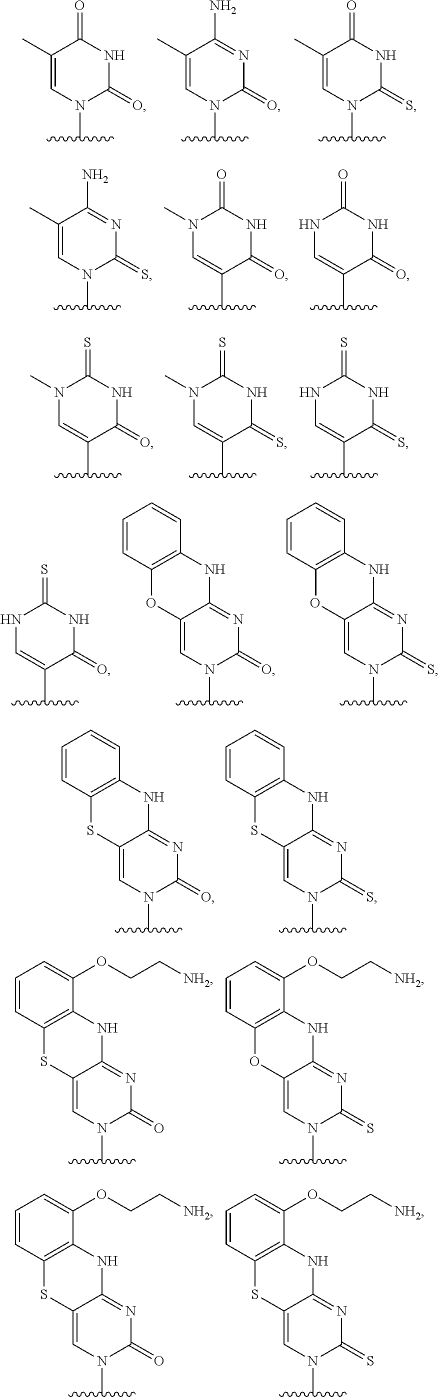

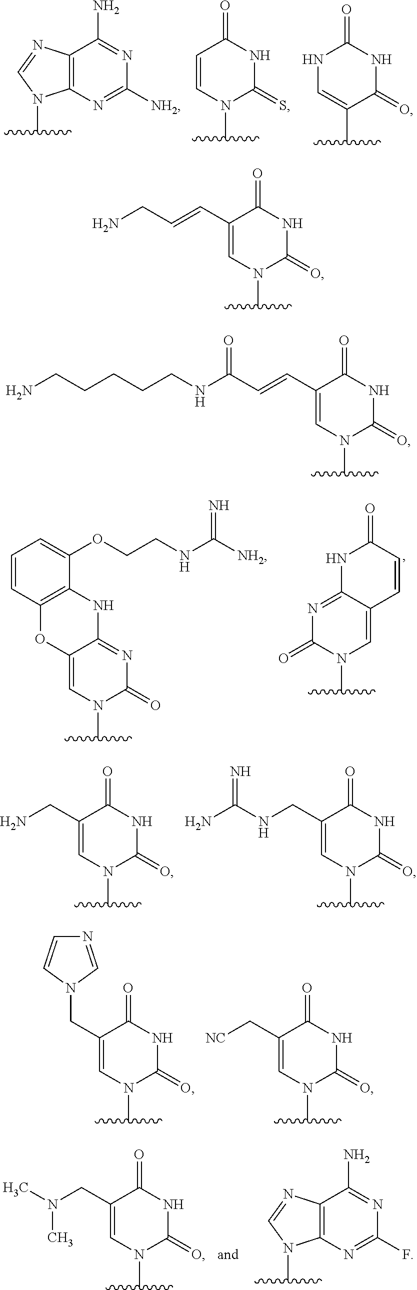

More preferably, B represents (i)

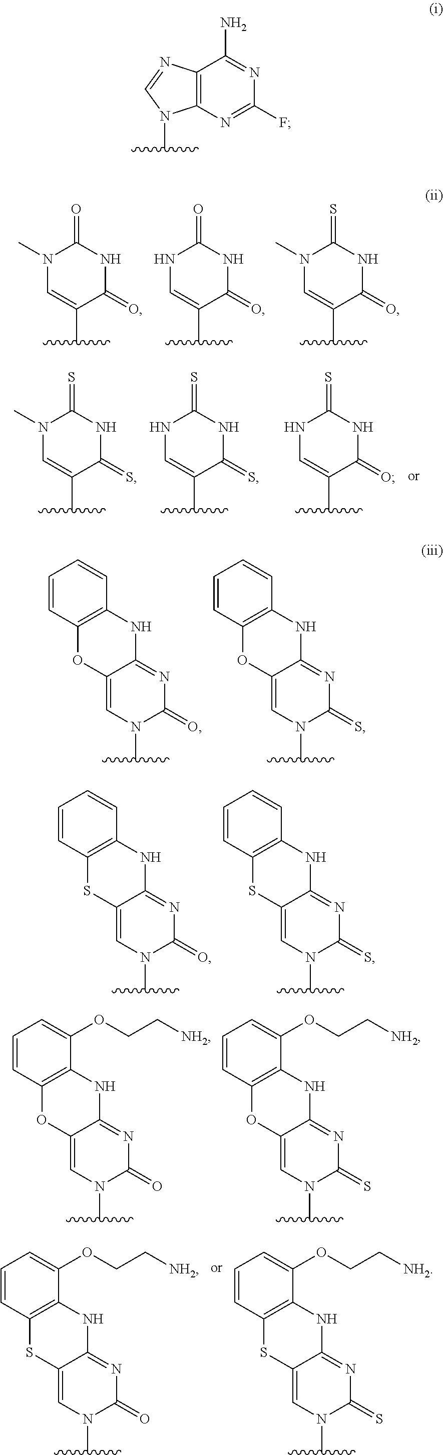

Another preferred embodiment is directed to nucleosides where R³ is an alkyl group, such as methyl, and the nucleobase is

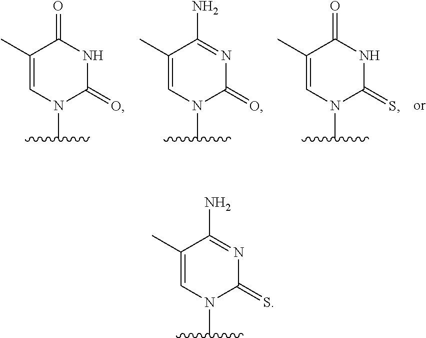

The invention also relates to an oligonucleotide comprising the above-noted nucleoside. The oligonucleotide may contain at least one non-phosphodiester backbone linkage. Suitable non-phosphodiester backbone linkages include phosphorothioate, phosphorodithioate, alkyl-phosphonate and phosphoramidate backbone linkage. The oligonucleotide may be an RNAi agent, an antisense, an antagomir, a microRNA, a pre-microRNA, an antimir, a ribozyme or an aptamer.

The invention also relates an RNAi agent comprising at least one nucleoside. The RNAi agent may be double stranded. In various embodiments, only one strand, such as the sense strand or the antisense strand containes the modified nucleoside. In other embodiments, both strands contain at least one nucleoside. The nucleoside may be the same or different in the two strands. The RNAi agent may also be single stranded. As known in the art, the RNAi agent may contain a hairpin structure. Other aspects relating the nucleosides, nucleotides, oligonucleotides, RNAi agents or methods of inhibiting gene expression are discussed throughout this disclosure and apply to the preferred compounds discussed above.

More than one modified nucleoside can be present in the oligonucleotide, for example an oligonucleotide may comprise 1, 2, 3, 4, 5, 6, 7, 8, 9 or 10 modified nucleosides described above. When more than one modified nucleosides are present in an oligonucleotide, all modified nucleosides may be the same or all different, or some same and some different or a combination thereof.

The modified nucleosides can be placed at any position in the oligonucleotide. By way of example, a modified nucleoside may only occur at a 3′ or 5′ terminal position, may only occur in the internal region, may only occur in a terminal regions, e.g. at a position on a terminal nucleotide or in the last 2, 3, 4, 5, or 10 nucleotides of an oligonucleotide. A modified nucleoside may occur in a double strand region, a single strand region, or in both. A modified nucleoside may occur only in the double strand region of an oligonucleotide or may only occur in a single strand region of an oligonucleotide.

When the oligonucleotide is an RNAi agent, e.g. a double stranded RNAi agent, the modified nucleoside may only be present in the sense strand or may only be present in the antisense strand. In one embodiment, both the sense and the antisense strands comprise at least one modified nucleoside described above. In one embodiment, only the sense strand comprises at least one modified nucleoside described above. In one embodiment, only the antisense strand comprises at least one modified nucleoside described above.

The backbone linkages between the modified nucleoside described above and rest of the oligonucleotide can be the canonical phosphodiester backbone linkage or a non-phosphodiester backbone linkage described below. In one embodiment, the linkages are chosen from a group consisting of phosphorothioate, phosphorodithioate, alkyl phosphonate and phosphoramidate backbone linkage. The oxygen at 5′-position of the modified nucleoside can be linked to the 2′-, 3′- or 5′-position of a nucleotide in the oligonucleotide. The oxygen at 3′-position of the modified nucleoside can be linked to the 2′-, 3′- or 5′-position of a nucleotide in the oligonucleotide.

In addition to the modified nucleosides, the oligonucleotides of the invention can further comprise one or more other modifications in the oligonucleotides. These further modifications include for example, but not limited to, modifications to the ribose sugar, modifications of the backbone phosphodiester linkages and modifications of the nucleobases.

In one embodiment, the oligonucleotide further comprises at least one nucleotide with a sugar modification. When more than one nucleotides with a sugar modification are present, all sugar modification may be the same or all different or a combination thereof.

In one embodiment, at least 2%, at least 5%, at least 7.5%, at least 10%, at least 20%, at least 30%, at least 40%, at least 50%, at least 60%, at least 70%, at least 80%, at least 90% or at least 95% of the nucleotides in the oligonucleotide comprise a sugar modification.

In one modification, all nucleotides in the oligonucleotide comprise a sugar modification.

In one embodiment, all the pyrimidine nucleotides in the oligonucleotide comprise a sugar modification.

In one embodiment, all the purine nucleotides in the oligonucleotide comprise a sugar modification.

In one embodiment, alternating nucleotides in the oligonucleotide comprise a sugar modification. In one embodiment, the first nucleotide on the 5′-end of the oligonucleotide comprises a sugar modification and then alternating nucleotides comprise a sugar modification. In one embodiment, the first nucleotide on the 5′-end of the oligonucleotide does not comprise a sugar modification and then alternating nucleotides comprise a sugar modification.

In one embodiment, when the oligonucleotide is double-stranded, juxtaposed nucleosides in the opposite strands both comprise a sugar modification.

In one embodiment, when the oligonucleotide is double-stranded, of the juxtaposed nucleosides in the opposite strands only one comprises a sugar modification.

In one embodiment, all the pyrimidine in the dinucleotide sequence motif 5′-pyrimidine-purine-3′ comprise a sugar modification.

In one embodiment, the sugar modification is at the 2′-position.

In one embodiment, the sugar modification is chosen from a group consisting of 2′-β-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH2-(4′-C) (LNA) and 2′-O—CH₂CH₂-(4′-C) (ENA).

In one embodiment, the oligonucleotide comprises at least one non-phosphodiester backbone linkage. The non-phosphodiester backbone may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini.

In one embodiment, non-phosphodiester backbone linkage is selected from a group consisting of phosphorothioate, phosphorodithioate, alkyl-phosphonate and phosphoramidate backbone linkage.

In one embodiment, the oligonucleotide comprises at least one ligand conjugate.

In one embodiment, the oligonucleotide comprises two or more ligand conjugates.

In one embodiment, the oligonucleotide is a double-stranded oligonucleotide.

In one embodiment, only one strand comprises the modified nucleoside.

In one embodiment, both strands comprise the modified nucleoside.

In one embodiment, the modified nucleoside is the same in the two strands.

In one embodiment, the modified nucleoside is different in the two strands.

In one embodiment, the oligonucleotide is a single-stranded oligonucleotide.

In one embodiment, the oligonucleotide has a hairpin structure.

In one embodiment, the oligonucleotide is an RNAi agent, an antisense, an antagomir, a microRNA, a pre-microRNA, an antimir, a ribozyme or an aptamer oligonucleotide.

In one embodiment, the RNAi agent is double stranded and only the sense strand comprises the modified nucleoside.

In one embodiment, the RNAi agent is double stranded and only the antisense strand comprises the modified nucleoside.

In one embodiment, the RNAi agent is double-stranded and both the sense and the antisness strands comprise at least one modified nucleoside.

In one embodiment, the modified nucleoside is the same in both the sense and the antisness strands.

In one embodiment, the sense and the antisense strands comprise different modified nucleosides.

### Oligonucleotides

In the context of this invention, the term “oligonucleotide” refers to a polymer or oligomer of nucleotide or nucleoside monomers consisting of naturally occurring bases, sugars and intersugar (backbone) linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The nucleic acids used herein can be single-stranded or double-stranded. A single stranded oligonucleotide may have double stranded regions and a double stranded oligonucleotide may have regions of single-stranded regions. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include, e.g., ssRNA, antisense oligonucleotides, ribozymes, microRNAs, aptamers, antagomirs, supermir, miRNA mimic, U1 adaptor, triplex-forming oligonucleotides and single-stranded RNAi agents.

Oligonucleotides of the present invention may be of various lengths. In particular embodiments, oligonucleotides may range from about 10 to 100 nucleotides in length. In various related embodiments, oligonucleotides, single-stranded, double-stranded, and triple-stranded, may range in length from about 10 to about 50 nucleotides, from about 20 to about 50 nucleotides, from about 15 to about 30 nucleotides, from about 20 to about 30 nucleotides in length.

The oligonucleotides of the invention may comprise any oligonucleotide modification described herein and below. In certain instances, it may be desirable to modify one or both strands of a dsRNA. In some cases, the two strands will include different modifications. Multiple different modifications can be included on each of the strands. The modifications on a given strand may differ from each other, and may also differ from the various modifications on other strands. For example, one strand may have a modification, e.g., a modification described herein, and a different strand may have a different modification, e.g., a different modification described herein. In other cases, one strand may have two or more different modifications, and the another strand may include a modification that differs from the at least two modifications on the other strand.

### Double-Stranded Oligonucleotides

In one embodiment, the invention provides double-stranded ribonucleic acid (dsRNA) molecules for inhibiting the expression of the target gene (alone or in combination with a second dsRNA for inhibiting the expression of a second target gene) in a cell or mammal, wherein the dsRNA comprises an antisense strand comprising a region of complementarity which is complementary to at least a part of an mRNA formed in the expression of the target gene, and wherein the region of complementarity is less than 30 nucleotides in length, generally 19-24 nucleotides in length, and wherein said dsRNA, upon contact with a cell expressing said target gene, inhibits the expression of said target gene. The dsRNA comprises two RNA strands that are sufficiently complementary to hybridize to form a duplex structure. Generally, the duplex structure is between 15 and 30, more generally between 18 and 25, yet more generally between 19 and 24, and most generally between 19 and 21 base pairs in length. In certain embodiments, longer dsRNAs of between 25 and 30 base pairs in length are preferred. In certain embodiments, shorter dsRNAs of between 10 and 15 base pairs in length are preferred. In another embodiment, the dsRNA is at least 21 nucleotides long and includes a sense RNA strand and an antisense RNA strand, wherein the antisense RNA strand is 25 or fewer nucleotides in length, and the duplex region of the dsRNA is 18-25 nucleotides in length, e.g., 19-24 nucleotides in length.

Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s).

In a preferred embodiment, the target gene is a human target gene.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above the dsRNAs of the invention can comprise at least one strand of a length of minimally 21 nt. It can be reasonably expected that shorter dsRNAs comprising a known sequence minus only a few nucleotides on one or both ends may be similarly effective as compared to the dsRNAs of the lengths described above. Hence, dsRNAs comprising a partial sequence of at least 15, 16, 17, 18, 19, 20, or more contiguous nucleotides, and differing in their ability to inhibit the expression of the target gene by not more than 5, 10, 15, 20, 25, or 30% inhibition from a dsRNA comprising the full sequence, are contemplated by the invention. Further dsRNAs that cleave within the target sequence can readily be made using the target gene sequence and the target sequence provided.

Double-stranded and single-stranded oligonucleotides that are effective in inducing RNA interference are also referred to as siRNA, RNAi agent and/or iRNA agent. These RNA interference inducing oligonucleotides associate with a cytoplasmic multi-protein complex known as RNAi-induced silencing complex (RISC). In many embodiments, single-stranded and double stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g. a target mRNA.

The present invention further includes RNAi agents that target within the sequence targeted by one of the agents of the present invention. As used herein a second RNAi agent is said to target within the sequence of a first RNAi agent if the second RNAi agent cleaves the message anywhere within the mRNA that is complementary to the antisense strand of the first RNAi agent. Such a second agent will generally consist of at least 15 contiguous nucleotides coupled to additional nucleotide sequences taken from the region contiguous to the selected sequence in the target gene.

The dsRNA of the invention can contain one or more mismatches to the target sequence. In a preferred embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of the target gene, the dsRNA generally does not contain any mismatch within the central 13 nucleotides. The methods described within the invention can be used to determine whether a dsRNA containing a mismatch to a target sequence is effective in inhibiting the expression of the target gene. Consideration of the efficacy of dsRNAs with mismatches in inhibiting expression of the target gene is important, especially if the particular region of complementarity in the target gene is known to have polymorphic sequence variation within the population.

In certain embodiment, the sense-strand comprises a mismatch to the antisense strand. In some embodiments, the mismatch is at the 5 nucleotides from the 3′-end, for example 5, 4, 3, 2, or 1 nucleotide from the end of the region of complementarity. In some embodiments, the mismatch is located in the target cleavage site region. In one embodiment, the sense strand comprises no more than 1, 2, 3, 4 or 5 mismatches to the antisense strand. In preferred embodiments, the sense strand comprises no more than 3 mismatches to the antisense strand.

In certain embodiments, the sense strand comprises a nucleobase modification, e.g. an optionally substituted natural or non-natural nucleobase, a universal nucleobase, in the target cleavage site region.

The “target cleavage site” herein means the backbone linkage in the target gene, e.g. target mRNA, or the sense strand that is cleaved by the RISC mechanism by utilizing the iRNA agent. And the “target cleavage site region” comprises at least one or at least two nucleotides on both side of the cleavage site. For the sense strand, the target cleavage site is the backbone linkage in the sense strand that would get cleaved if the sense strand itself was the target to be cleaved by the RNAi mechanism. The target cleavage site can be determined using methods known in the art, for example the 5′-RACE assay as detailed in Soutschek et al., Nature (2004) 432, 173-178. As is well understood in the art, the cleavage site region for a conical double stranded RNAi agent comprising two 21-nucleotides long strands (wherein the strands form a double stranded region of 19 consecutive basepairs having 2-nucleotide single stranded overhangs at the 3′-ends), the cleavage site region corresponds to postions 9-12 from the 5′-end of the sense strand.

In one embodiment, at least one end of the dsRNA has a single-stranded nucleotide overhang of 1 to 4, generally 1 or 2 nucleotides. In one embodiment, the single-stranded overhang has the sequence 5′-GCNN-3′, wherein N is independently for each occurrence, A, G, C, U, dT, dU or absent. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Generally, the antisense strand of the dsRNA has a nucleotide overhang at the 3′-end, and the 5′-end is blunt.

In one embodiment, the antisense strand of the dsRNA has 1-10 nucleotides overhangs each at the 3′ end and the 5′ end over the sense strand. In one embodiment, the sense strand of the dsRNA has 1-10 nucleotides overhangs each at the 3′ end and the 5′ end over the antisense strand.

The dsRNAs of the invention may comprise any oligonucleotide modification described herein and below. In certain instances, it may be desirable to modify one or both strands of a dsRNA. In some cases, the two strands will include different modifications. Multiple different modifications can be included on each of the strands. The modifications on a given strand may differ from each other, and may also differ from the various modifications on other strands. For example, one strand may have a modification, e.g., a modification described herein, and a different strand may have a different modification, e.g., a different modification described herein. In other cases, one strand may have two or more different modifications, and the another strand may include a modification that differs from the at least two modifications on the other strand.

In one embodiment, the dsRNA is chemically modified to enhance stability. In one preferred embodiment, one or more of the nucleotides in the overhang is replaced with a nucleoside thiophosphate.

The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of an dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNAduplex. Activation of RNase H, therefore, results in cleavage of the RNA target, thereby greatly enhancing the efficiency of dsRNA inhibition of gene expression.

The present invention also includes dsRNAs wherein the two strands are linked together. The two strands can be linked together by a polynucleotide linker such as (dT)n; wherein n is 4-10, and thus forming a hairpin. The two strands can also be linked together by a non-nucleosidic linker, e.g. a linker described herein. It will be appreciated by one of skill in the art that any oligonucleotide chemical modifications or variations describe herein can be used in the polynucleotide linker.

Hairpin RNAi agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In certain embodiments, the overhangs are 2-3 nucleotides in length.

The RNAi agents of the invention can target more than one RNA region. For example, an RNAi agent can include a first and second sequence that are sufficiently complementary to each other to hybridize. The first sequence can be complementary to a first target RNA region and the second sequence can be complementary to a second target RNA region. The first and second sequences of the RNAi agent can be on different RNA strands, and the mismatch between the first and second sequences can be less than 50%, 40%, 30%, 20%, 10%, 5%, or 1%. The first and second sequences of the RNAi agent can be on the same RNA strand, and in a related embodiment more than 50%, 60%, 70%, 80%, 90%, 95%, or 1% of the RNAi agent can be in bimolecular form. The first and second sequences of the RNAi agent can be fully complementary to each other.

The first target RNA region can be encoded by a first gene and the second target RNA region can encoded by a second gene, or the first and second target RNA regions can be different regions of an RNA from a single gene. The first and second sequences can differ by at least 1 nucleotide.

The first and second target RNA regions can be on transcripts encoded by first and second sequence variants, e.g., first and second alleles, of a gene. The sequence variants can be mutations, or polymorphisms, for example. The first target RNA region can include a nucleotide substitution, insertion, or deletion relative to the second target RNA region, or the second target RNA region can a mutant or variant of the first target region.

The first and second target RNA regions can comprise viral or human RNA regions. The first and second target RNA regions can also be on variant transcripts of an oncogene or include different mutations of a tumor suppressor gene transcript. In addition, the first and second target RNA regions can correspond to hot-spots for genetic variation.

The double stranded oligonucleotides can be optimized for RNA interference by increasing the propensity of the duplex to disassociate or melt (decreasing the free energy of duplex association), in the region of the 5′ end of the antisense strand This can be accomplished, e.g., by the inclusion of modifications or modified nucleosides which increase the propensity of the duplex to disassociate or melt in the region of the 5′ end of the antisense strand. It can also be accomplished by inclusion of modifications or modified nucleosides or attachment of a ligand that increases the propensity of the duplex to disassociate of melt in the region of the 5′ end of the antisense strand. While not wishing to be bound by theory, the effect may be due to promoting the effect of an enzyme such as helicase, for example, promoting the effect of the enzyme in the proximity of the 5′ end of the antisense strand.

Modifications which increase the tendency of the 5′ end of the antisense strand in the duplex to dissociate can be used alone or in combination with other modifications described herein, e.g., with modifications which decrease the tendency of the 3′ end of the antisense in the duplex to dissociate Likewise, modifications which decrease the tendency of the 3′ end of the antisense in the duplex to dissociate can be used alone or in combination with other modifications described herein, e.g., with modifications which increase the tendency of the 5′ end of the antisense in the duplex to dissociate.

Nucleic acid base pairs can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; I:C is preferred over G:C (I=inosine); mismatches, e.g., non-canonical or other than canonical pairings are preferred over canonical (A:T, A:U, G:C) pairings; pairings which include a universal base are preferred over canonical pairings.

It is preferred that pairings which decrease the propensity to form a duplex are used at 1 or more of the positions in the duplex at the 5′ end of the antisense strand. The terminal pair (the most 5′ pair in terms of the antisense strand), and the subsequent 4 base pairing positions (going in the 3′ direction in terms of the antisense strand) in the duplex are preferred for placement of modifications to decrease the propensity to form a duplex. More preferred are placements in the terminal most pair and the subsequent 3, 2, or 1 base pairings. It is preferred that at least 1, and more preferably 2, 3, 4, or 5 of the base pairs from the 5′-end of antisense strand in the duplex be chosen independently from the group of: A:U, G:U, I:C, mismatched pairs, e.g., non-canonical or other than canonical pairings or pairings which include a universal base. In a preferred embodiment at least one, at least 2, or at least 3 base-pairs include a universal base.

Modifications or changes which promote dissociation are preferably made in the sense strand, though in some embodiments, such modifications/changes will be made in the antisense strand.

Nucleic acid base pairs can also be ranked on the basis of their propensity to promote stability and inhibit dissociation or melting (e.g., on the free energy of association or dissociation of a particular pairing, the simplest approach is to examine the pairs on an individual pair basis, though next neighbor or similar analysis can also be used). In terms of promoting duplex stability: G:C is preferred over A:U, Watson-Crick matches (A:T, A:U, G:C) are preferred over non-canonical or other than canonical pairings, analogs that increase stability are preferred over Watson-Crick matches (A:T, A:U, G:C), e.g. 2-amino-A:U is preferred over A:U, 2-thio U or 5 Me-thio-U:A, are preferred over U:A, G-clamp (an analog of C having 4 hydrogen bonds):G is preferred over C:G, guanadinium-G-clamp:G is preferred over C:G, psuedo uridine:A, is preferred over U:A, sugar modifications, e.g., 2′ modifications, e.g., 2′F, ENA, or LNA, which enhance binding are preferred over non-modified moieties and can be present on one or both strands to enhance stability of the duplex.

It is preferred that pairings which increase the propensity to form a duplex are used at 1 or more of the positions in the duplex at the 3′ end of the antisense strand. The terminal pair (the most 3′ pair in terms of the antisense strand), and the subsequent 4 base pairing positions (going in the 5′ direction in terms of the antisense strand) in the duplex are preferred for placement of modifications to decrease the propensity to form a duplex. More preferred are placements in the terminal most pair and the subsequent 3, 2, or 1 base pairings. It is preferred that at least 1, and more preferably 2, 3, 4, or 5 of the pairs of the recited regions be chosen independently from the group of: G:C, a pair having an analog that increases stability over Watson-Crick matches (A:T, A:U, G:C), 2-amino-A:U, 2-thio U or 5 Me-thio-U:A, G-clamp (an analog of C having 4 hydrogen bonds):G, guanadinium-G-clamp:G, psuedo uridine:A, a pair in which one or both subunits has a sugar modification, e.g., a 2′ modification, e.g., 2′F, ENA, or LNA, which enhance binding. In some embodiments, at least one, at least, at least 2, or at least 3, of the base pairs promote duplex stability.

In a preferred embodiment the at least one, at least 2, or at least 3, of the base pairs are a pair in which one or both subunits has a sugar modification, e.g., a 2′ modification, e.g., 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA) and 2′-O—CH₂CH₂-(4′-C) (ENA), which enhances binding.

G-clamps and guanidinium G-clamps are discussed in the following references: Holmes and Gait, “The Synthesis of 2′-O-Methyl G-Clamp Containing Oligonucleotides and Their Inhibition of the HIV-1 Tat-TAR Interaction,” Nucleosides, Nucleotides & Nucleic Acids, 22:1259-1262, 2003; Holmes et al., “Steric inhibition of human immunodeficiency virus type-1 Tat-dependent trans-activation in vitro and in cells by oligonucleotides containing 2′-O-methyl G-clamp ribonucleoside analogues,” Nucleic Acids Research, 31:2759-2768, 2003; Wilds, et al., “Structural basis for recognition of guanosine by a synthetic tricyclic cytosine analogue: Guanidinium G-clamp,” Helvetica Chimica Acta, 86:966-978, 2003; Rajeev, et al., “High-Affinity Peptide Nucleic Acid Oligomers Containing Tricyclic Cytosine Analogues,” Organic Letters, 4:4395-4398, 2002; Ausin, et al., “Synthesis of Amino- and Guanidino-G-Clamp PNA Monomers,” Organic Letters, 4:4073-4075, 2002; Maier et al., “Nuclease resistance of oligonucleotides containing the tricyclic cytosine analogues phenoxazine and 9-(2-aminoethoxy)-phenoxazine (“G-clamp”) and origins of their nuclease resistance properties,” Biochemistry, 41:1323-7, 2002; Flanagan, et al., “A cytosine analog that confers enhanced potency to antisense oligonucleotides,” Proceedings Of The National Academy Of Sciences Of The United States Of America, 96:3513-8, 1999.

As is discussed above, an oligonucleotide can be modified to both decrease the stability of the antisense 5′ end of the duplex and increase the stability of the antisense 3′ end of the duplex. This can be effected by combining one or more of the stability decreasing modifications in the antisense 5′ end of the duplex with one or more of the stability increasing modifications in the antisense 3′ end of the duplex.

### Single-Stranded Oligonucleotides

The single-stranded oligonucleotides of the present invention also comprise nucleotide sequence that is substantially complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. The region of complementarity is less than 30 nucleotides in length, and at least 15 nucleotides in length. Generally, the single stranded oligonucleotides are 10 to 25 nucleotides in length (e.g., 11, 12, 13, 14, 15, 16, 18, 19, 20, 21, 22, 23, or 24 nucleotides in length). In one embodiment the strand is 25-30 nucleotides. Single strands having less than 100% complementarity to the target mRNA, RNA or DNA are also embraced by the present invention. These single-stranded oligonucleotides are also referred to as antisense, antagomir and antimir oligonucleotides.

The single-stranded oligonucleotide can hybridize to a complementary RNA, and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H. Degradation of the target RNA prevents translation.

Single-stranded oligonucleotides, including those described and/or identified as microRNAs or mirs which may be used as targets or may serve as a template for the design of oligonucleotides of the invention are taught in, for example, Esau, et al. US Publication No. 2005/0261218 (U.S. Ser. No. 10/909,125) entitled “Oligomeric compounds and compositions for use in modulation small non-coding RNAs” the entire contents of which is incorporated herein by reference. It will be appreciated by one of skill in the art that any oligonucleotide chemical modifications or variations describe herein also apply to single stranded oligonucleotides.

MicroRNAs (miRNAs or mirs) are a highly conserved class of small RNA molecules that are transcribed from DNA in the genomes of plants and animals, but are not translated into protein. Pre-microRNAs are processed into miRNAs. Processed microRNAs are single stranded 17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111.

Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety. Antagomir RNAs may be synthesized using standard solid phase oligonucleotide synthesis protocols. See U.S. patent application Ser. Nos. 11/502,158 and 11/657,341 (the disclosure of each of which are incorporated herein by reference).

An antagomir can include ligand-conjugated monomer subunits and monomers for oligonucleotide synthesis. Exemplary monomers are described in U.S. application Ser. No. 10/916,185, filed on Aug. 10, 2004. An antagomir can have a ZXY structure, such as is described in PCT Application No. PCT/US2004/07070 filed on Mar. 8, 2004. An antagomir can be complexed with an amphipathic moiety. Exemplary amphipathic moieties for use with oligonucleotide agents are described in PCT Application No. PCT/US2004/07070, filed on Mar. 8, 2004.

Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000). Aptamers may be RNA or DNA based. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

miRNA mimics. miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs). miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

Supermir. A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. An supermir featured in the invention can have secondary structure, but it is substantially single-stranded under physiological conditions. An supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

Antimir or miRNA inhibitor. The terms “antimir” “microRNA inhibitor”, “miR inhibitor”, or “inhibitor” are synonymous and refer to oligonucleotides or modified oligonucleotides that interfere with the ability of specific miRNAs. In general, the inhibitors are nucleic acid or modified nucleic acids in nature including oligonucleotides comprising RNA, modified RNA, DNA, modified DNA, locked nucleic acids (LNAs), or any combination of the above. Modifications include 2′ modifications (including 2′-0 alkyl modifications and 2′ F modifications) and internucleotide modifications (e.g. phosphorothioate modifications) that can affect delivery, stability, specificity, intracellular compartmentalization, or potency. In addition, miRNA inhibitors can comprise conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors comprise contain one or more sequences or portions of sequences that are complementary or partially complementary with the mature strand (or strands) of the miRNA to be targeted, in addition, the miRNA inhibitor may also comprise additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

Viral miRNAs. In small-sized viral genomes, miRNAs offer an efficient strategy for specific inactivation of host cell defense factors. miRNAs have been cloned from herpes viruses, Epstein-Ban virus, human cytomegalovirus, Kaposi's sarcoma-associated virus and are predicted in the genomes of double-stranded DNA (dsDNA) viruses such as herpes simplex virus 1 and 2, variola and vaccinia virus, molluscum contagiosum virus, and human adenoviruses and in the genomes of the single-stranded RNA viruses, measles virus and yellow fever virus. Clear evidence of the importance of miRNA in the viral life cycle has been shown for the simian virus 40 (SV40). The miRNAs from the circular dsDNA SV40 are perfectly complementary to the early viral mRNAs coding for T antigen. The miRNAs accumulate late in infection and reduce the expression of viral T antigens. The cells with miRNAs are less sensitive than cells without miRNA to lysis by cytotoxic T cells and trigger less cytokine production by such cells.

Viral suppression of silencing. Given the huge number of host miRNAs and the potential for complementarity with viral genomes, viruses have an incentive to interfere with the silencing pathway. There is evidence that some viruses inhibit the RNAi pathway. Primate foamy virus type 1 (PFV-1) expresses a protein that sequesters siRNAs. Adenovirus-infected cells accumulate polymerase III transcripts known as virus-associated RNAs (VA RNAs). The VA RNAs appear to inhibit the RNAi pathway through binding of Dicer as well as through competition for the nuclear export factor.

In contrast, hepatitis C virus (HCV) exploits a cellular miRNA to maintain viral abundance. The liver-specific miRNA, miR-122, discussed above in the section on antagomirs is required for high levels of HCV replication. In Huh7 cells containing replicating HCV genomes, the sequestration of miR-122 with antagomirs resulted in a reduction in the amount of HCV RNA. There are two potential binding sites for miR-122 in the HCV RNA: one in the 3′ UTR and the other in the 5′ UTR. Surprisingly, experiments showed that a direct interaction occurs between miR-122 and the 5′ UTR site of HCV RNA. The regulation is likely to occur during replication, rather than during translation or by interference with RNA stability. The binding of miR-122 might allow a conformational rearrangement in the 5′ UTR of the HCV RNA that allows replication to proceed or components of the miRISC that are recruited by miR-122 might be required for viral replication. Current treatments for HCV are often ineffective and a compound directed against conserved sequences of a cellular target such as miR-122 could be attractive. The work described above that dissected the role of miR-122 in cellular metabolism is a first step toward development of an miR-directed therapeutic.

A recent study with HSV-1 showed that the latency-associated transcript (LAT) gene is responsible for survival of HSV-1 in infected neurons. The microRNA generated from the exon 1 region of the HSV-1 LAT gene (miR-LAT) down-regulates two important genes: transforming-growth factor-13 (TGF-β) and SMAD3. Both genes are involved in the TGF-β pathway and can either inhibit cell proliferation or induce cell death. Antagomir approaches to inhibition of miR-LAT could be a viable therapeutic approach for abolishing HSV-1 in neurons.

### U1 Adaptor

U1 adaptor inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant MoI Biol 49:77-95). Nucleotides 2-11 of the 5′ end of U1 snRNA base pair bind with the 5'ss of the pre mRNA. In one embodiment, oligonucleotides of the invention are U1 adaptors. In one embodiment, the U1 adaptor can be administered in combination with at least one other iRNA agent.

### Other Oligonucleotides

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest., 2000, 106: 1071-1075, which is expressly incorporated by reference herein, in its entirety.

### Oligonucleotide Modifications

Unmodified oligonucleotides may be less than optimal in some applications, e.g., unmodified oligonucleotides can be prone to degradation by e.g., cellular nucleases. Nucleases can hydrolyze nucleic acid phosphodiester bonds. However, chemical modifications to one or more of the above oligonucleotide components can confer improved properties, and, e.g., can render oligonucleotides more stable to nucleases.

Modified nucleic acids and nucleotide surrogates can include one or more of:

(i) alteration, e.g., replacement, of one or both of the non-linking phosphate oxygens and/or of one or more of the linking phosphate oxygens in the phosphodiester backbone linkage.

(ii) alteration, e.g., replacement, of a constituent of the ribose sugar, e.g., of the 2′ hydroxyl on the ribose sugar;

(iii) wholesale replacement of the phosphate moiety with “dephospho” linkers;

(iv) modification or replacement of a naturally occurring base with a non-natural base;

(v) replacement or modification of the ribose-phosphate backbone;

(vi) modification of the 3′ end or 5′ end of the oligonucleotide, e.g., removal, modification or replacement of a terminal phosphate group or conjugation of a moiety, e.g., a fluorescently labeled moiety, to either the 3′ or 5′ end of oligonucleotide; and

(vii) modification of the sugar (e.g., six membered rings).

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

As oligonucleotides are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications may be incorporated in a single oligonucleotide or even at a single nucleoside within an oligonucleotide.

In some cases the modification will occur at all of the subject positions in the oligonucleotide but in many, and in fact in most cases it will not. By way of example, a modification may only occur at a 3′ or 5′ terminal position, may only occur in the internal region, may only occur in a terminal regions, e.g. at a position on a terminal nucleotide or in the last 2, 3, 4, 5, or 10 nucleotides of an oligonucleotide. A modification may occur in a double strand region, a single strand region, or in both. A modification may occur only in the double strand region of an oligonucleotide or may only occur in a single strand region of an oligonucleotide. E.g., a phosphorothioate modification at a non-bridging oxygen position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

A modification described herein may be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is particularly preferred, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Specific modifications are discussed in more detail below.

### The Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following: S, Se, BR₃ (R is hydrogen, alkyl, aryl), C (i.e. an alkyl group, an aryl group, etc.), H, NR₂ (R is hydrogen, alkyl, aryl), or OR (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotides diastereomers. Thus, while not wishing to be bound by theory, modifications to both non-bridging oxygens, which eliminate the chiral center, e.g. phosphorodithioate formation, may be desirable in that they cannot produce diastereomer mixtures. Thus, the non-bridging oxygens can be independently any one of S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replcament with carbon is preferred. When the bridging oxygen is the 5′-oxygen of a nucleoside, replcament with nitrogen is preferred.

### Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non-phosphodiester backbone linkage.”

### Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the mophilino, cyclobutyl, pyrrolidine and peptide nucleic acid (PNA) nucleoside surrogates. A preferred surrogate is a PNA surrogate.

### Sugar Modifications

An oligonucleotide can include modification of all or some of the sugar groups of the nucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, 0(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the 1′ position on the sugar, e.g., alpha-nucleosides. Oligonucleotides can also include “abasic” sugars, which lack a nucleobase at C-1′. These abasic sugars can also be further containing modifications at one or more of the constituent sugar atoms. Oligonucleotides can also contain one or more sugars that are in the L form, e.g. L-nucleosides.

Preferred substitutents are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-β-DMAOE), 2′-O-dimethylaminopropyl (2′-O-DMAP) and 2′-O-dimethylaminoethyloxyethyl (2′-O-DMAEOE).

### Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a dsRNA, this array can substitute for a hairpin RNA loop in a hairpin-type RNA agent.

Terminal modifications useful for modulating activity include modification of the 5′ end with phosphate or phosphate analogs. E.g., in preferred embodiments antisense strands of dsRNAs, are 5′ phosphorylated or include a phosphoryl analog at the 5′ prime terminus. 5′-phosphate modifications include those which are compatible with RISC mediated gene silencing. Modifications at the 5′-terminal end can also be useful in stimulating or inhibiting the immune system of a subject. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-beta-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH2-), 5′-alkyletherphosphonates (R=alkylether=methoxymethyl (MeOCH₂—), ethoxymethyl, etc., e.g. RP(OH)(O)—O-5′-). Other embodiments include replacement of oxygen/sulfur with BH₃, BH₃⁻ and/or Se.

Terminal modifications can also be useful for monitoring distribution, and in such cases the preferred groups to be added include fluorophores, e.g., fluorscein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

### Nucleobases

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, or any O-alkylated or N-alkylated derivatives thereof;

Further purines and pyrimidines include those disclosed in U.S. Pat. No. 3,687,808, hereby incorporated by reference, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

### Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

Placement within an Oligonucleotide

Some modifications may preferably be included on an oligonucleotide at a particular location, e.g., at an internal position of a strand, or on the 5′ or 3′ end of an oligonucleotide. A preferred location of a modification on an oligonucleotide, may confer preferred properties on the agent. For example, preferred locations of particular modifications may confer optimum gene silencing properties, or increased resistance to endonuclease or exonuclease activity.

One or more nucleotides of an oligonucleotide may have a 2′-5′ linkage. One or more nucleotides of an oligonucleotide may have inverted linkages, e.g. 3′-3′, 5′-5′,2′-2′ or 2′-3′ linkages.

A double-stranded oligonucleotide may include at least one 5′-uridine-adenine-3′ (5′-UA-3′) dinucleotide wherein the uridine is a 2′-modified nucleotide, or a terminal 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

### GENERAL REFERENCES

The oligoribonucleotides and oligoribonucleosides used in accordance with this invention may be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

### Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

### Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

### Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleo sides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 11972, 1991. Carbamate replacements are described in Stirchak, E. P. Nucleic Acids Res. 1989, 17, 6129.

### Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. No. 5,539,083.

### Terminal Modification References

Terminal modifications are described in Manoharan, M. et al. Antisense and Nucleic Acid Drug Development 12, 103-128 (2002) and references therein.

### Bases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references are disclosed in the above section on base modifications

### Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art may additionally or alternatively be employed. It is also known to use similar techniques to prepare other oligonucleotides, such as the phosphorothioates, phosphorodithioates and alkylated derivatives. The double-stranded oligonucleotide compounds of the invention may be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides may be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having .beta.-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

### Ligands

A wide variety of entities can be coupled to the oligonucleotides of the present invention. Preferred moieties are ligands, which are coupled, preferably covalently, either directly or indirectly via an intervening tether.

In preferred embodiments, a ligand alters the distribution, targeting or lifetime of the molecule into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ligands providing enhanced affinity for a selected target are also termed targeting ligands.

Some ligands can have endosomolytic properties. The endosomolytic ligands promote the lysis of the endosome and/or transport of the composition of the invention, or its components, from the endosome to the cytoplasm of the cell. The endosomolytic ligand may be a polyanionic peptide or peptidomimetic which shows pH-dependent membrane activity and fusogenicity. In certain embodiments, the endosomolytic ligand assumes its active conformation at endosomal pH. The “active” conformation is that conformation in which the endosomolytic ligand promotes lysis of the endosome and/or transport of the composition of the invention, or its components, from the endosome to the cytoplasm of the cell. Exemplary endosomolytic ligands include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic component may contain a chemical group (e.g., an amino acid) which will undergo a change in charge or protonation in response to a change in pH. The endosomolytic component may be linear or branched. Exemplary primary sequences of peptide based endosomolytic ligands are shown in Table 1.

TABLE 1
List of peptides with endosomolytic activity.

| Name | Sequence (N to C) | Ref. |
|---|---|---|
| GALA | AALEALAEALEALAEALEALAEAAAAGGC | 1 |
| EALA | AALAEALAEALAEALAEALAEALAAAAGGC | 2 |
| | ALEALAEALEALAEA | 3 |
| INF-7 | GLFEAIEGFIENGWEGMIWDYG | 4 |
| Inf HA-2 | GLFGAIAGFIENGWEGMIDGWYG | 5 |
| diINF-7 | GLF EAI EGFI ENGW EGMI DGWYGC | 5 |
| | GLF EAI EGFI ENGW EGMI DGWYGC | |
| diINF3 | GLF EAI EGFI ENGW EGMI DGGC | 6 |
| | GLF EAI EGFI ENGW EGMI DGGC | |
| GLF | GLFGALAEALAEALAEHLAEALAEALEALAAGGSC | 6 |
| GALA-INF3 | GLFEAIEGFIENGWEGLAEALAEALEALAAGGSC | 6 |
| INF-5 | GLF EAI EGFI ENGW EGnI DG K | 4 |
| | GLF EAI EGFI ENGW EGnI DG | |

n, norleucine
References
1. Subbarao et al., Biochemistry, 1987, 26: 2964-2972.
2. Vogel et al., J. Am. Chem. Soc., 1996, 118: 1581-1586
3. Turk, M. J., Reddy, J. A. et al. (2002). Characterization of a novel pH-sensitive peptide that enhances drug release from folate-targeted liposomes at endosomal pHs. Biochim. Biophys. Acta 1559, 56-68.
4. Plank, C. Oberhauser, B. Mechtler, K. Koch, C. Wagner, E. (1994). The influence of endosome-disruptive peptides on gene transfer using synthetic virus-like gene transfer systems, J. Biol. Chem. 269 12918-12924.
5. Mastrobattista, E., Koning, G. A. et al. (2002). Functional characterization of an endosome-disruptive peptide and its application in cytosolic delivery of immunoliposome-entrapped proteins. J. Biol. Chem. 277, 27135-43.
6. Oberhauser, B., Plank, C. et al. (1995). Enhancing endosomal exit of nucleic acids using pH-sensitive viral fusion peptides. Deliv. Strategies Antisense Oligonucleotide Ther. 247-66.

Ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer. Table 2 shows some examples of targeting ligands and their associated receptors.

TABLE 2
Targeting Ligands and their associated receptors

| Liver Cells | Ligand | Receptor |
|---|---|---|
| 1) Parenchymal | Galactose | ASGP-R |
| Cell (PC) | | (Asiologlycoprotein |
| (Hepatocytes) | | receptor) |
| | Gal NAc | ASPG-R |
| | (n-acetyl-galactosamine) | Gal NAc Receptor |
| | Lactose | |
| | Asialofetuin | ASPG-r |
| 2) Sinusoidal | Hyaluronan | Hyaluronan receptor |
| Endothelial | | |
| Cell (SEC) | | |
| | Procollagen | Procollagen receptor |
| | Negatively charged | Scavenger receptors |
| | molecules | |
| | Mannose | Mannose receptors |
| | N-acetyl Glucosamine | Scavenger receptors |
| | Immunoglobulins | Fc Receptor |
| | LPS | CD14 Receptor |
| | Insulin | Receptor mediated |
| | | transcytosis |
| | Transferrin | Receptor mediated |
| | | transcytosis |
| | Albumins | Non-specific |
| | Sugar-Albumin | |
| | conjugates | |
| | Mannose-6-phosphate | Mannose-6-phosphate |
| | | receptor |
| 3) Kupffer | Mannose | Mannose receptors |
| Cell (KC) | | |
| | Fucose | Fucose receptors |
| | Albumins | Non-specific |
| | Mannose-albumin | |
| | conjugates | |

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-κB.

The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the oligonucleotide into the cell by activating an inflammatory response, for example. Exemplary ligands that would have such an effect include tumor necrosis factor alpha (TNFalpha), interleukin-1 beta, or gamma interferon.

In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HAS, low density lipoprotein (LDL) and high-density lipoprotein (HDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long (see Table 3, for example).

TABLE 3
Exemplary Cell Permeation Peptides.

| Cell Permeation | | |
|---|---|---|
| Peptide | Amino acid Sequence | Reference |
| Penetratin | RQIKIWFQNRRMKWKK | Derossi et al., J. Biol. |
| | | Chem. 269: 10444, 1994 |
| Tat fragment | GRKKRRQRRRPPQC | Vives et al., J. Biol. Chem., |
| (48-60) | | 272: 16010, 1997 |
| Signal Sequence- | GALFLGWLGAAGSTMGAWSQPKKKR | Chaloin et al., Biochem. |
| based peptide | KV | Biophys. Res. Commun., |
| | | 243: 601, 1998 |
| PVEC | LLIILRRRIRKQAHAHSK | Elmquist et al., Exp. Cell |
| | | Res., 269: 237, 2001 |
| Transportan | GWTLNSAGYLLKINLKALAALAKKIL | Pooga et al., FASEB J., |
| | | 12: 67, 1998 |
| Amphiphilic | KLALKLALKALKAALKLA | Oehlke et al., Mol. Ther., |
| model peptide | | 2: 339, 2000 |
| Arg₉ | RRRRRRRRR | Mitchell et al., J. Pept. Res., |
| | | 56: 318, 2000 |
| Bacterial cell wall | KFFKFFKFFK | |
| permeating | | |
| LL-37 | LLGDFFRKSKEKIGKEFKRIVQRIKDFL | |
| | RNLVPRTES | |
| Cecropin P1 | SWLSKTAKKLENSAKKRISEGIAIAIQG | |
| | GPR | |
| α-defensin | ACYCRIPACIAGERRYGTCIYQGRLWA | |
| | FCC | |
| b-defensin | DHYNCVSSGGQCLYSACPIFTKIQGTC | |
| | YRGKAKCCK | |
| Bactenecin | RKCRIVVIRVCR | |
| PR-39 | RRRPRPPYLPRPRPPPFFPPRLPPRIPPGF | |
| | PPRFPPRFPGKR-NH2 | |
| Indolicidin | ILPWKWPWWPWRR-NH2 | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIVVFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a tumor cell, such as an endothelial tumor cell or a breast cancer tumor cell (Zitzmann et al., Cancer Res., 62:5139-43, 2002). An RGD peptide can facilitate targeting of an iRNA agent to tumors of a variety of other tissues, including the lung, kidney, spleen, or liver (Aoki et al., Cancer Gene Therapy 8:783-787, 2001). Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing αᵥβ₃ (Haubner et al., Jour. Nucl. Med., 42:326-336, 2001).

Peptides that target markers enriched in proliferating cells can be used, e.g., RGD containing peptides and peptidomimetics can target cancer cells, in particular cells that exhibit an Iᵥθ₃ integrin. Thus, one could use RGD peptides, cyclic peptides containing RGD, RGD peptides that include D-amino acids, as well as synthetic RGD mimics. In addition to RGD, one can use other moieties that target the Iᵥ-θ₃ integrin ligand. Generally, such ligands can be used to control proliferating cells and angiogeneis. Preferred conjugates of this type lignads that targets PECAM-1, VEGF, or other cancer gene, e.g., a cancer gene described herein.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In one embodiment, a targeting peptide can be an amphipathic α-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an apatamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycarboxylates, polyacations, masked oligo or poly cations or anions, acetals, polyacetals, ketals/polyketyals, orthoesters, polymers with masked or unmasked cationic or anionic charges, dendrimers with masked or unmasked cationic or anionic charges.

PK modulator stands for pharmacokinetic modulator. PK modulator include lipophiles, bile acids, steroids, phospholipid analogues, peptides, protein binding agents, PEG, vitamins etc. Examplary PK modulator include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable to the present invention as ligands (e.g. as PK modulating ligands).

In addition, aptamers that bind serum components (e.g. serum proteins) are also amenable to the present invention as PK modulating ligands.

Other ligand conjugates amenable to the invention are described in copending applications U.S. Ser. No. 10/916,185, filed Aug. 10, 2004; U.S. Ser. No. 10/946,873, filed Sep. 21, 2004; U.S. Ser. No. 10/833,934, filed Aug. 3, 2007; U.S. Ser. No. 11/115,989 filed Apr. 27, 2005 and U.S. Ser. No. 11/944,227 filed Nov. 21, 2007, which are incorporated by reference in their entireties for all purposes.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. In preferred embodiments, the ligand is attached to the oligonucleotides via an intervening tether, e.g. a carrier described herein. The ligand or tethered ligand may be present on a monomer when said monomer is incorporated into the growing strand. In some embodiments, the ligand may be incorporated via coupling to a “precursor” monomer after said “precursor” monomer has been incorporated into the growing strand. For example, a monomer having, e.g., an amino-terminated tether (i.e., having no associated ligand), e.g., TAP-(CH₂)ₙNH₂ may be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction may be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands. In some embodiments, a double-stranded iRNA agent contains a ligand conjugated to the sense strand. In other embodiments, a double-stranded iRNA agent contains a ligand conjugated to the antisense strand.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; and 6,559,279, each of which is herein incorporated by reference.

### Linkers

In certain embodiments, the covalent linkages between the oligonucleotide and other components, e.g. a ligand or a ligand carrying monomer may be mediated by a linker. This linker may be cleavable or non-cleavable, depending on the application. In certain embodiments, a cleavable linker may be used to release the nucleic acid after transport to the desired target. The intended nature of the conjugation or coupling interaction, or the desired biological effect, will determine the choice of linker group.

The term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In one embodiment, the linker is —[(P-Q-R)_{q}—X—(P′-Q′-R′)_{q′}]_{q″}-T—, wherein:

P, R, T, P′ and R′ are each independently for each occurrence absent, CO, NH, O, S, OC(O), NHC(O), CH₂, CH₂NH, CH₂O; NHCH(R^{a})C(O), —C(O)—CH(R^{a})—NH—, C(O)-(optionally substituted alkyl)-NH—, CH═N—O,

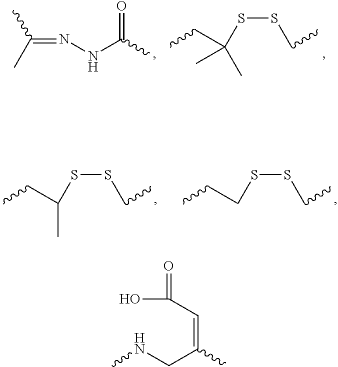

or heterocyclyl;

Q and Q′ are each independently for each occurrence absent, —(CH₂)ₙ—, —C(R¹⁰⁰)(R²⁰⁰)(CH₂)ₙ—,—(CH₂)ₙC(R¹⁰⁰)(R²⁰⁰)—, —(CH₂CH₂O)ₘCH₂CH₂—, or —(CH₂CH₂O)ₘCH₂CH₂NH—;

X is absent or a cleavable linking group;

R^{a} is H or an amino acid side chain;

R¹⁰⁰ and R²⁰⁰ are each independently for each occurrence H, CH₃, OH, SH or N(R^{X})₂;

R^{X} is independently for each occurrence H, methyl, ethyl, propyl, isopropyl, butyl or benzyl;

q, q′ and q″ are each independently for each occurrence 0-20 and wherein the repeating unit can be the same or different;

n is independently for each occurrence 1-20; and

m is independently for each occurrence 0-50.

In one embodiment, the linker comprises at least one cleavable linking group.

In certain embodiments, the linker is a branched linker. The branchpoint of the branched linker may be at least trivalent, but may be a tetravalent, pentavalent or hexavalent atom, or a group presenting such multiple valencies. In certain embodiments, the branchpoint is, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or a glycerol derivative.

### Cleavable Linking Groups

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids. These candidates can be evaluated using methods analogous to those described above.

### Formulations

For ease of exposition the formulations, compositions and methods in this section are discussed largely with regard to RNAi agents. It may be understood, however, that these formulations, compositions and methods can be practiced with other oligonucleotides of the invention, e.g., antisense, antagomir, aptamer and ribozyme, and such practice is within the invention.

A formulated RNAi composition can assume a variety of states. In some examples, the composition is at least partially crystalline, uniformly crystalline, and/or anhydrous (e.g., less than 80, 50, 30, 20, or 10% water). In another example, the RNAi is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the RNAi composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An RNAi preparation can be formulated in combination with another agent, e.g., another therapeutic agent or an agent that stabilizes the RNAi agent, e.g., a protein that complex with RNAi agent to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the RNAi preparation includes another RNAi agent, e.g., a second RNAi that can mediated RNAi with respect to a second gene, or with respect to the same gene. Still other preparation can include at least 3, 5, ten, twenty, fifty, or a hundred or more different RNAi species. Such RNAi agents can mediate RNAi with respect to a similar number of different genes.

In one embodiment, the RNAi preparation includes at least a second therapeutic agent (e.g., an agent other than RNA or DNA). For example, an RNAi composition for the treatment of a viral disease, e.g., HIV, might include a known antiviral agent (e.g., a protease inhibitor or reverse transcriptase inhibitor). In another example, an RNAi agent composition for the treatment of a cancer might further comprise a chemotherapeutic agent.

Exemplary formulations are discussed below:

### Liposomes

The oligonucleotides of the invention, e.g. antisense, antagomir, aptamer, ribozyme and RNAi agent can be formulated in liposomes. As used herein, a liposome is a structure having lipid-containing membranes enclosing an aqueous interior. Liposomes may have one or more lipid membranes. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 Liposomes with several nonconcentric membranes, i.e., several smaller vesicles contained within a larger vesicle, are termed multivesicular vesicles.

Liposomes may further include one or more additional lipids and/or other components such as cholesterol. Other lipids may be included in the liposome compositions for a variety of purposes, such as to prevent lipid oxidation, to stabilize the bilayer, to reduce aggregation during formation or to attach ligands onto the liposome surface. Any of a number of lipids may be present, including amphipathic, neutral, cationic, and anionic lipids. Such lipids can be used alone or in combination.

Additional components that may be present in a lipsomes include bilayer stabilizing components such as polyamide oligomers (see, e.g., U.S. Pat. No. 6,320,017), peptides, proteins, detergents, lipid-derivatives, such as PEG conjugated to phosphatidylethanolamine, PEG conjugated to phosphatidic acid, PEG conjugated to ceramides (see, U.S. Pat. No. 5,885,613), PEG conjugated dialkylamines and PEG conjugated 1,2-diacyloxypropan-3-amines.

Liposome can include components selected to reduce aggregation of lipid particles during formation, which may result from steric stabilization of particles which prevents charge-induced aggregation during formation. Suitable components that reduce aggregation include, but are not limited to, polyethylene glycol (PEG)-modified lipids, monosialoganglioside Gml, and polyamide oligomers (“PAO”) such as (described in U.S. Pat. No. 6,320,017). Exemplary suitable PEG-modified lipids include, but are not limited to, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Particularly preferred are PEG-modified diacylglycerols and dialkylglycerols. Other compounds with uncharged, hydrophilic, steric-barrier moieties, which prevent aggregation during formation, like PEG, Gml, or ATTA, can also be coupled to lipids to reduce aggregation during formation. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids). It should be noted that aggregation preventing compounds do not necessarily require lipid conjugation to function properly. Free PEG or free ATTA in solution may be sufficient to prevent aggregation. If the liposomes are stable after formulation, the PEG or ATTA can be dialyzed away before administration to a subject.

Neutral lipids, when present in the liposome composition, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in liposomes described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ are preferred. In another group of embodiments, lipids with mono or diunsaturated fatty acids with carbon chain lengths in the range of C₁₄ to C₂₂ are used. Additionally, lipids having mixtures of saturated and unsaturated fatty acid chains can be used. Preferably, the neutral lipids used in the present invention are DOPE, DSPC, POPC, DMPC, DPPC or any related phosphatidylcholine. The neutral lipids useful in the present invention may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Cationic lipids, when present in the liposome composition, can be any of a number of lipid species which carry a net positive charge at about physiological pH. Such lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N²-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N, N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), 5-carboxyspermylglycine diocaoleyamide (“DOGS”), and dipalmitoylphosphatidylethanolamine 5-carboxyspermyl-amide (“DPPES”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other cationic lipids suitable for lipid particle formation are described in WO98/39359, WO96/37194. Other cationic lipids suitable for liposome formation are described in U.S. Provisional applications No. 61/018,616 (filed Jan. 2, 2008), No. 61/039,748 (filed Mar. 26, 2008), No. 61/047,087 (filed Apr. 22, 2008) and No. 61/051,528 (filed May 21-2008), all of which are incorporated by reference in their entireties for all purposes.

Anionic lipids, when present in the liposome composition, can be any of a number of lipid species which carry a net negative charge at about physiological pH. Such lipids include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and O-acyloxyacids, can also be used. Additionally, such amphipathic lipids can be readily mixed with other lipids, such as triglycerides and sterols.

Also suitable for inclusion in the liposome compostions of the present invention are programmable fusion lipids. Liposomes containing programmable fusion lipids have little tendency to fuse with cell membranes and deliver their payload until a given signal event occurs. This allows the liposome to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the liposome membrane over time. By the time the liposome is suitably distributed in the body, it has lost sufficient cloaking agent so as to be fusogenic. With other signal events, it is desirable to choose a signal that is associated with the disease site or target cell, such as increased temperature at a site of inflammation.

A liposome can also include a targeting moiety, e.g., a targeting moiety that is specific to a cell type or tissue. Targeting of liposomes with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fl (1995). Other targeting moieties, such as ligands, cell surface receptors, glycoproteins, vitamins (e.g., riboflavin), aptamers and monoclonal antibodies, can also be used. The targeting moieties can include the entire protein or fragments thereof. Targeting mechanisms generally require that the targeting agents be positioned on the surface of the liposome in such a manner that the targeting moiety is available for interaction with the target, for example, a cell surface receptor.

In one approach, a targeting moiety, such as receptor binding ligand, for targeting the liposome is linked to the lipids forming the liposome. In another approach, the targeting moiety is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)). A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002). Other lipids conjugated with targeting moieties are described in U.S. provisional application No. 61/127,751 (filed May 14, 2008) and PCT application No. PCT/US2007/080331 (filed Oct. 3, 2007), all of which are incorporated by reference in their entireties for all purposes.

A liposome composition of the invention can be prepared by a variety of methods that are known in the art. See e.g., U.S. Pat. No. 4,235,871, No. 4,897,355 and No. 5,171,678; published PCT applications WO 96/14057 and WO 96/37194; Felgner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

For example, a liposome composition of the invention can be prepared by first dissolving the lipid components of a liposome in a detergent so that micelles are formed with the lipid component. The detergent can have a high critical micelle concentration and maybe nonionic. Exemplary detergents include, but are not limited to, cholate, CHAPS, octylglucoside, deoxycholate and lauroyl sarcosine. The RNAi agent preparation e.g., an emulsion, is then added to the micelles that include the lipid components. After condensation, the detergent is removed, e.g., by dialysis, to yield a liposome containing the RNAi agent. If necessary a carrier compound that assists in condensation can be added during the condensation reaction, e.g., by controlled addition. For example, the carrier compound can be a polymer other than a nucleic acid (e.g., spermine or spermidine). To favor condensation, pH of the mixture can also be adjusted.

In another example, liposomes of the present invention may be prepared by diffusing a lipid derivatized with a hydrophilic polymer into preformed liposome, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the RNAi agent is first dispersed by sonication in a lysophosphatidylcholine or other low CMC surfactant (including polymer grafted lipids). The resulting micellar suspension of RNAi agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323.

Other suitable formulations for RNAi agents are described in PCT application No. PCT/US2007/080331 (filed Oct. 3, 2007) and U.S. Provisional applications No. 61/018,616 (filed Jan. 2, 2008), No. 61/039,748 (filed Mar. 26, 2008), No. 61/047,087 (filed Apr. 22, 2008) and No. 61/051,528 (filed May 21-2008), all of which are incorporated by reference in their entireties for all purposes.

### Micelles and Other Membranous Formulations

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

As defined herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C6-C20. Examples are polyethylene-glycolized fatty glycerides and polyethylene glycols.

Exemplary amphiphilic carriers include, but are not limited to, lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Mixed micelle formulation suitable for delivery through transdermal membranes may be prepared by mixing an aqueous solution of the RNAi composition, an alkali metal C₈ to C₂₂ alkyl sulphate, and an amphiphilic carrier. The amphiphilic carrier may be added at the same time or after addition of the alkali metal alkyl sulphate. Mixed micelles will form with substantially any kind of mixing of the ingredients but vigorous mixing in order to provide smaller size micelles.

In one method a first micelle composition is prepared which contains the RNAi composition and at least the alkali metal alkyl sulphate. The first micelle composition is then mixed with at least three amphiphilic carriers to form a mixed micelle composition. In another method, the micelle composition is prepared by mixing the RNAi composition, the alkali metal alkyl sulphate and at least one of the amphiphilic carriers, followed by addition of the remaining micelle amphiphilic carriers, with vigorous mixing.

Phenol and/or m-cresol may be added to the mixed micelle composition to stabilize the formulation and protect against bacterial growth. Alternatively, phenol and/or m-cresol may be added with the amphiphilic carriers. An isotonic agent such as glycerin may also be added after formation of the mixed micelle composition.

For delivery of the micelle formulation as a spray, the formulation can be put into an aerosol dispenser and the dispenser is charged with a propellant, such as hydrogen-containing chlorofluorocarbons, hydrogen-containing fluorocarbons, dimethyl ether, diethyl ether and HFA 134a (1,1,1,2 tetrafluoroethane).

### Emulsions

The oligonucleotides of the present invention may be prepared and formulated as emulsions. Emulsions are typically heterogenous systems of one liquid dispersed in another in the form of droplets (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials is also included in emulsion formulations and contributes to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of FLiPs are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Lipid Particles

It has been shown that cholesterol-conjugated sRNAis bind to HDL and LDL lipoprotein particles which mediate cellular uptake upon binding to their respective receptors. Both high-density lipoproteins (HDL) and low density lipoproteins (LDL) play a critical role in cholesterol transport. HDL directs sRNAi delivery into liver, gut, kidney and steroidogenic organs, whereas LDL targets sRNAi primarily to liver (Wolfrum et al. Nature Biotechnology Vol. 25 (2007)). Thus in one aspect the invention provides formulated lipid particles (FLiPs) comprising (a) an oligonucleotide of the invention, e.g., antisense, antagomir, aptamer, ribozyme and an RNAi agent, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated.

The stoichiometry of oligonucleotide to the lipid component may be 1:1. Alternatively the stoichiometry may be 1:many, many:1 or many:many, where many is greater than 2.

The FLiP may comprise triacylglycerol, phospholipids, glycerol and one or several lipid-binding proteins aggregated, admixed or associated via a lipophilic linker molecule with a single- or double-stranded oligonucleotide, wherein said FLiP has an affinity to heart, lung and/or muscle tissue. Surprisingly, it has been found that due to said one or several lipid-binding proteins in combination with the above mentioned lipids, the affinity to heart, lung and/or muscle tissue is very specific. These FLiPs may therefore serve as carrier for oligonucleotides. Due to their affinity to heart, lung and muscle cells, they may specifically transport the oligonucleotides to these tissues. Therefore, the FLiPs according to the present invention may be used for many severe heart, lung and muscle diseases, for example myocarditis, ischemic heart disease, myopathies, cardiomyopathies, metabolic diseases, rhabdomyosarcomas.

One suitable lipid component for FLiP is Intralipid. Intralipid® is a brand name for the first safe fat emulsion for human use. Intralipid® 20% (a 20% intravenous fat emulsion) is a sterile, non-pyrogenic fat emulsion prepared for intravenous administration as a source of calories and essential fatty acids. It is made up of 20% soybean oil, 1.2% egg yolk phospholipids, 2.25% glycerin, and water for injection. Intralipid® 10% is made up of 10% soybean oil, 1.2% egg yolk phospholipids, 2.25% glycerin, and water for injection. It is further within the present invention that other suitable oils, such as saflower oil, may serve to produce the lipid component of the FLiP.

In one embodiment of the invention is a FLiP comprising a lipid particle comprising 15-25% triacylglycerol, about 1-2% phospholipids and 2-3% glycerol, and one or several lipid-binding proteins.

In another embodiment of the invention the lipid particle comprises about 20% triacylglycerol, about 1.2% phospholipids and about 2.25% glycerol, which corresponds to the total composition of Intralipid, and one or several lipid-binding proteins.

Another suitable lipid component for FLiPs is lipoproteins, for example isolated lipoproteins or more preferably reconstituted lipoprotieins. Liporoteins are particles that contain both proteins and lipids. The lipids or their derivatives may be covalently or non-covalently bound to the proteins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins).

Methods of producing reconstituted lipoproteins have been described in scientific literature, for example see A. Jones, Experimental Lung Res. 6, 255-270 (1984), U.S. Pat. No. 4,643,988 and No. 5,128,318, PCT publication WO87/02062, Canadian patent No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

The most frequently used lipid for reconstitution is phosphatidyl choline, extracted either from eggs or soybeans. Other phospholipids are also used, also lipids such as triglycerides or cholesterol. For reconstitution the lipids are first dissolved in an organic solvent, which is subsequently evaporated under nitrogen. In this method the lipid is bound in a thin film to a glass wall. Afterwards the apolipoproteins and a detergent, normally sodium cholate, are added and mixed. The added sodium cholate causes a dispersion of the lipid. After a suitable incubation period, the mixture is dialyzed against large quantities of buffer for a longer period of time; the sodium cholate is thereby removed for the most part, and at the same time lipids and apolipoproteins spontaneously form themselves into lipoproteins or so-called reconstituted lipoproteins. As alternatives to dialysis, hydrophobic adsorbents are available which can adsorb detergents (Bio-Beads SM-2, Bio Rad; Amberlite XAD-2, Rohm & Haas) (E. A. Bonomo, J. B. Swaney, J. Lipid Res., 29, 380-384 (1988)), or the detergent can be removed by means of gel chromatography (Sephadex G-25, Pharmacia). Lipoproteins can also be produced without detergents, for example through incubation of an aqueous suspension of a suitable lipid with apolipoproteins, the addition of lipid which was dissolved in an organic solvent, to apolipoproteins, with or without additional heating of this mixture, or through treatment of an apoA-I-lipid-mixture with ultrasound. With these methods, starting, for example, with apoA-I and phosphatidyl choline, disk-shaped particles can be obtained which correspond to lipoproteins in their nascent state. Normally, following the incubation, unbound apolipoproteins and free lipid are separated by means of centrifugation or gel chromatography in order to isolate the homogeneous, reconstituted lipoproteins particles.

Phospholipids used for reconstituted lipoproteins can be of natural origin, such as egg yolk or soybean phospholipids, or synthetic or semisynthetic origin. The phospholipids can be partially purified or fractionated to comprise pure fractions or mixtures of phosphatidyl cholines, phosphatidyl ethanolamines, phosphatidyl inositols, phosphatidic acids, phosphatidyl serines, sphingomyelin or phosphatidyl glycerols. According to specific embodiments of the present invention it is preferred to select phospholipids with defined fatty acid radicals, such as dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and combinations thereof, and the like phosphatidyl cholines with defined acyl groups selected from naturally occurring fatty acids, generally having 8 to 22 carbon atoms. According to a specific embodiment of the present invention phosphatidyl cholines having only saturated fatty acid residues between 14 and 18 carbon atoms are preferred, and of those dipalmitoyl phosphatidyl choline is especially preferred.

Other phospholipids suitable for reconstitution with lipoproteins include, e.g., phosphatidylcholine, phosphatidylglycerol, lecithin, b, g-dipalmitoyl-a-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, and the like. Non-phosphorus containing lipids may also be used in the liposomes of the compositions of the present invention. These include, e.g., stearylamine, docecylamine, acetyl palmitate, fatty acid amides, and the like.

Besides the phospholipids, the lipoprotein may comprise, in various amounts at least one nonpolar component which can be selected among pharmaceutical acceptable oils (triglycerides) exemplified by the commonly employed vegetabilic oils such as soybean oil, safflower oil, olive oil, sesame oil, borage oil, castor oil and cottonseed oil or oils from other sources like mineral oils or marine oils including hydrogenated and/or fractionated triglycerides from such sources. Also medium chain triglycerides (MCT-oils, e.g. Miglyol®), and various synthetic or semisynthetic mono-, di- or triglycerides, such as the defined nonpolar lipids disclosed in WO 92/05571 may be used in the present invention as well as acetylated monoglycerides, or alkyl esters of fatty acids, such isopropyl myristate, ethyl oleate (see EP 0 353 267) or fatty acid alcohols, such as oleyl alcohol, cetyl alcohol or various nonpolar derivatives of cholesterol, such as cholesterol esters.

One or more complementary surface active agent can be added to the reconstituted lipoproteins, for example as complements to the characteristics of amphiphilic agent or to improve its lipid particle stabilizing capacity or enable an improved solubilization of the protein. Such complementary agents can be pharmaceutically acceptable non-ionic surfactants which preferably are alkylene oxide derivatives of an organic compound which contains one or more hydroxylic groups. For example ethoxylated and/or propoxylated alcohol or ester compounds or mixtures thereof are commonly available and are well known as such complements to those skilled in the art. Examples of such compounds are esters of sorbitol and fatty acids, such as sorbitan monopalmitate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cethyl ether, as well as polyoxyethylene castor oil or hydrogenated castor oil derivatives and polyglycerine fatty acid esters. Suitable non-ionic surfactants, include, but are not limited to various grades of Pluronic®, Poloxamer®, Span®, Tween®, Polysorbate®, Tyloxapol®, Emulphor® or Cremophor® and the like. The complementary surface active agents may also be of an ionic nature, such as bile duct agents, cholic acid or deoxycholic their salts and derivatives or free fatty acids, such as oleic acid, linoleic acid and others. Other ionic surface active agents are found among cationic lipids like C10-C24: alkylamines or alkanolamine and cationic cholesterol esters.

In the final FLiP, the oligonucleotide component is aggregated, associated or admixed with the lipid components via a lipophilic moiety. This aggregation, association or admixture may be at the surface of the final FLiP formulation. Alternatively, some integration of any of a portion or all of the lipophilic moiety may occur, extending into the lipid particle. Any lipophilic linker molecule that is able to bind oligonucleotides to lipids can be chosen. Examples include pyrrolidine and hydroxyprolinol.

The process for making the lipid particles comprises the steps of:

a) mixing a lipid components with one or several lipophile (e.g. cholesterol) conjugated oligonucleotides that may be chemically modified;
b) fractionating this mixture;
c) selecting the fraction with particles of 30-50 nm, preferably of about 40 nm in size.

Alternatively, the FLiP can be made by first isolating the lipid particles comprising triacylglycerol, phospholipids, glycerol and one or several lipid-binding proteins and then mixing the isolated particles with >2-fold molar excess of lipophile (e.g. cholesterol) conjugated oligonucleotide. The steps of fractionating and selecting the particles are deleted by this alternative process for making the FLiPs.

Other pharmacologically acceptable components can be added to the FLiPs when desired, such as antioxidants (exemplified by alpha-tocopherol) and solubilization adjuvants (exemplified by benzylalcohol).

### Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### Polymers

Hydrophilic polymers suitable for use in the formulations of the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

### Surfactants

The above discussed formulation may also include one or more surfactants. Surfactants find wide application in formulations such as emulsions (including microemulsions) and liposomes. The use of surfactants in drug products, formulations and in emulsions has been reviewed (Rieger, in “Pharmaceutical Dosage Forms,” Marcel Dekker, Inc., New York, N.Y., 1988, p. 285). Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Nonionic surfactants include, but are not limited to, nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

Anionic surfactants include, but are not limited to, carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

Cationic surfactants include, but are not limited to, quaternary ammonium salts and ethoxylated amines. The quaternary ammonium salts are the most used members of this class.

Amphoteric surfactants include, but are not limited to, acrylic acid derivatives, substituted alkylamides, N-alkylbetaines and phosphatides.

A surfactant may also be selected from any suitable aliphatic, cycloaliphatic or aromatic surfactant, including but not limited to biocompatible lysophosphatidylcholines (LPCs) of varying chain lengths (for example, from about C₁₄ about C₂₀). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

### Penetration Enhancers

In one embodiment, the formulations of the present invention employ various penetration enhancers to affect the efficient delivery of RNAi agents to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

### Pharmaceutical Compositions

In another aspect, the present invention provides pharmaceutically acceptable compositions which comprise a therapeutically-effective amount of one or more of the oligonucleotides described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Formulations for ocular administration can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” 0 and B books, Corvallis, Ore., U.S.A., 1977).

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

The phrases “2′-modification” and “2′-modified nucleotide” refer to a nucleotide unit having a sugar moiety, for example a ribosyl moiety, that is modified at the 2′-position such that the hydroxyl group (2′-OH) is replaced by, for example, —F, —H, —CH₃, —CH₂CH₃, —OCH₃, —OCH₂CH₃, —OCH₂CH₂OMe, —OCH₂C(═O)NHMe, —OCH₂-(4′-C) (a so-called “LNA sugar modification”), or —OCH₂CH₂-(4′-C) (a so-called “ENA sugar modification”). For example, the phrases “2′-fluoro modification” and “2′-fluoro modified nucleotide” refer to a nucleotide unit having a sugar moiety, for example a ribosyl moiety, that is modified at the 2′-position such that the hydroxyl group (2′-OH) is replaced by a fluoro group (2′-F). U.S. Pat. Nos. 6,262,241, and 5,459,255 (both of which are incorporated by reference), are drawn to, inter alia, methods of synthesizing 2′-fluoro modified nucleotides and oligonucleotides.

The phrase “antisense strand” as used herein, refers to a polynucleotide that is substantially or 100% complementary to a target nucleic acid of interest. An antisense strand may comprise a polynucleotide that is RNA, DNA or chimeric RNA/DNA. For example, an antisense strand may be complementary, in whole or in part, to a molecule of messenger RNA, an RNA sequence that is not mRNA (e.g., tRNA, rRNA and hnRNA) or a sequence of DNA that is either coding or non-coding. The phrase “antisense strand” includes the antisense region of both polynucleotides that are formed from two separate strands, as well as unimolecular polynucleotides that are capable of forming hairpin structures. The terms “antisense strand” and “guide strand” are used interchangeably herein.

The phrase “sense strand” refers to a polynucleotide that has the same nucleotide sequence, in whole or in part, as a target nucleic acid such as a messenger RNA or a sequence of DNA. The sense strand is not incorporated into the functional riboprotein RISC. The terms “sense strand” and “passenger strand” are used interchangeably herein.

The term “duplex” includes a region of complementarity between two regions of two or more polynucleotides that comprise separate strands, such as a sense strand and an antisense strand, or between two regions of a single contiguous polynucleotide.

As used herein, “specifically hybridizable” and “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., a to t, a to u, c to g), or in any other manner that allows for the formation of stable duplexes. “Perfect complementarity” or 100% complementarity refers to the situation in which each nucleotide unit of one polynucleotide strand can hydrogen bond with each nucleotide unit of a second polynucleotide strand. Less than perfect complementarity refers to the situation in which some, but not all, nucleotide units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an RNAi agent against a given target causes an unintended affect by interacting either directly or indirectly with another mRNA sequence, a DNA sequence or a cellular protein or other moiety. For example, an “off-target effect” may occur when there is a simultaneous degradation of other transcripts due to partial homology or complementarity between that other transcript and the sense and/or antisense strand of a double-strand RNAi agent.

The phrase “first 5′ terminal nucleotide” includes first 5′ terminal antisense nucleotides and first 5′ terminal antisense nucleotides. “First 5′ terminal antisense nucleotide” refers to the nucleotide of the antisense strand that is located at the 5′ most position of that strand with respect to the bases of the antisense strand that have corresponding complementary bases on the sense strand. Thus, in a double stranded polynucleotide that is made of two separate strands, it refers to the 5′ most base other than bases that are part of any 5′ overhang on the antisense strand. When the first 5′ terminal antisense nucleotide is part of a hairpin molecule, the term “terminal” refers to the 5′ most relative position within the antisense region and thus is the 5″ most nucleotide of the antisense region. The phrase “first 5” terminal sense nucleotide” is defined in reference to the sense nucleotide. In molecules comprising two separate strands, it refers to the nucleotide of the sense strand that is located at the 5′ most position of that strand with respect to the bases of the sense strand that have corresponding complementary bases on the antisense strand. Thus, in a double stranded polynucleotide that is made of two separate strands, it is the 5′ most base other than bases that are part of any 5′ overhang on the sense strand.

In certain embodiments, an siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, In certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

The term “nucleoside” includes a ribonucleoside or a deoxyribonucleoside or modified form thereof, as well as an analog thereof. Nucleosides include species that comprise purines, e.g., adenine, hypoxanthine, guanine, and their derivatives and analogs, as well as pyrimidines, e.g., cytosine, uracil, thymine, and their derivatives and analogs.

The term “pseudouracil” or “5-uracil” refers to

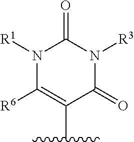

when R¹, R³ and R⁶ are hydrogen. Substituted pseudouracils are defined as follows: when R¹ is not hydrogen, it is a 1-substituted pseudouracil; when R³ is not hydrogen, it is a 3-substituted pseudouracil; and when R⁶ is not hydrogen, it is a 6-substituted uracil. The terms “2-(thio)pseudouracil”,

“4-(thio)pseudouracil” and “2,4-(dithio)psuedouracil” refer to

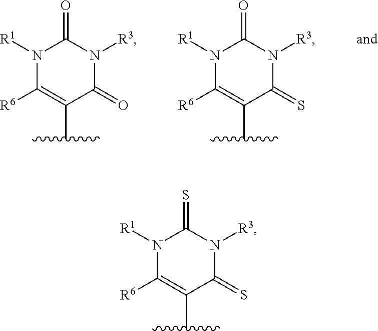

respectively. Suitable R¹, R³ and R⁶ include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

The term “1,3-(diaza)-2-(oxo)-phenoxazin-1-yl” as used herein refers to

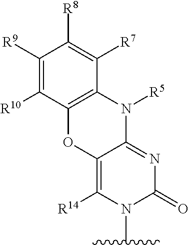

when R⁵, R⁷, R⁸, R⁹, R¹⁰, and R¹⁴ are hydrogen. Substituted 1,3-(diaza)-2-(oxo)-phenoxazin-1-yl are numbered as are the R groups (e.g., a 7-substituted 1,3-(diaza)-2-(oxo)-phenoxazin-1-yl is a compound wherein R⁷ is not hydrogen). The term “1-(aza)-2-(thio)-3-(aza)-phenoxazin-1-yl” as used herein refers to

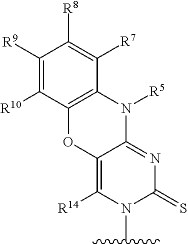

when R⁵, R⁷, R⁸, R⁹, R¹⁰, and R¹⁴ are hydrogen. The term “1,3-(diaza)-2-(oxo)-phenthiazin-1-yl” as used herein refers to

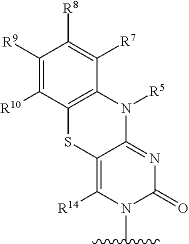

when R⁵, R⁷, R⁸, R⁹, R¹⁰, and R¹⁴ are hydrogen. The term “1-(aza)-2-(thio)-3-(aza)-phenthiazin-1-yl” as used herein refers to

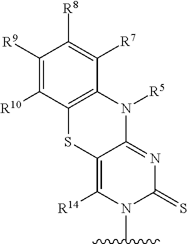

when R⁵, R⁷, R⁸, R⁹, R¹⁰, and R¹⁴ are hydrogen. Suitable R⁵, R⁷ to R¹⁰, and R¹⁴ include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

As used herein, “aminoalkylhydroxy” refers to —O-alkyl-amino (e.g., —OCH₂CH₂NH₂). As used herein, “guanidiniumalkylhydroxy” refers to —O-alkyl-guanidinium (e.g., —OCH₂CH₂N(H)C(═NH)NH₂).

As used herein, “aminocarbonylethylenyl” refers to

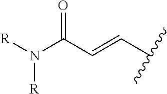

(e.g., when both R are hydrogen,

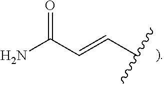

As used herein, “aminoalkylaminocarbonylethylenyl” refers to

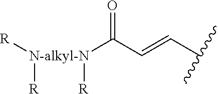

(e.g., when all three R are hydrogen,

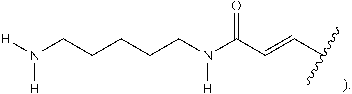

Suitable R include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

The term “1,3,5-(triaza)-2,6-(dioxa)-naphthalene” as used herein refers to

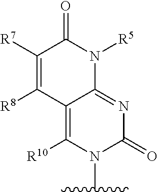

when R⁵, R⁷, R⁸ and R¹⁰ are hydrogen. Suitable R⁵, R⁷, R⁸ and R¹⁰ include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

The phrase “pharmaceutically acceptable carrier or diluent” includes compositions that facilitate the introduction of nucleic acid therapeutics such as siRNA, dsRNA, dsDNA, shRNA, microRNA, antimicroRNA, antagomir, antimir, antisense, aptamer or dsRNA/DNA hybrids into a cell and includes but is not limited to solvents or dispersants, coatings, anti-infective agents, isotonic agents, and agents that mediate absorption time or release of the inventive polynucleotides and double stranded polynucleotides. The phrase “pharmaceutically acceptable” includes approval by a regulatory agency of a government, for example, the U.S. federal government, a non-U.S. government, or a U.S. state government, or inclusion in a listing in the U.S. Pharmacopeia or any other generally recognized pharmacopeia for use in animals, including in humans.

The terms “silence” and “inhibit the expression of” and related terms and phrases, refer to the at least partial suppression of the expression of a gene targeted by an siRNA or siNA, as manifested by a reduction of the amount of mRNA transcribed from the target gene which may be isolated from a first cell or group of cells in which the target gene is transcribed and which has or have been treated such that the expression of the target gene is inhibited, as compared to a second cell or group of cells substantially identical to the first cell or group of cells but which has or have not been so treated (i.e., control cells).

The term “halo” refers to any radical of fluorine, chlorine, bromine or iodine. The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, C₁-C₁₀ indicates that the group may have from 1 to 10 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “oxo” refers to an oxygen atom, which forms a carbonyl when attached to carbon, an N-oxide when attached to nitrogen, and a sulfoxide or sulfone when attached to sulfur. The term “thio” refers to a sulfur atom, which forms a thiocarbonyl when attached to a carbon.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

In many cases, protecting groups are used during preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, for example, are disclosed by Beaucage et al. (Tetrahedron 1992, 48, 2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y, 1991.

Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett. 1994, 35, 7821; Verhart and Tesser, Rec. Tray. Chim. Pays-Bas 1987, 107, 621).

Additional amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the present invention.

### Evaluation of Candidate RNAs

One can evaluate a candidate RNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added.

### Kits

In certain other aspects, the invention provides kits that include a suitable container containing a pharmaceutical formulation of an RNAi agent. In certain embodiments the individual components of the pharmaceutical formulation may be provided in one container. Alternatively, it may be desirable to provide the components of the pharmaceutical formulation separately in two or more containers, e.g., one container for an RNAi agent preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### EXAMPLES

The invention now being generally described, it will be more readily understood by reference to the following examples which are included merely for purposes of illustration of certain aspects and embodiments of the present invention, and are not intended to limit the invention.

### Example 1

### General Procedures

Thin-Layer Chromatography (TLC) was conducted on glass plates pre-coated with a 0.25-mm layer of silica gel 60 F-254 (Merck). The compounds were visualized either by exposure to UV light or by spraying with 5% H₂SO₄ and 0.2% p-anisaldehyde in a solution of ethanol and heating or both. Solutions were concentrated under reduced pressure at less than 40° C. The silica gel used for column chromatography was Merck Analyzed (230-400 mesh). ¹H-NMR spectra were recorded at 30° C. on a 400 MHz spectrometer. The values of δ(ppm) are given relative to the signal (δ 0) for internal Me₄Si for solutions in CDCl₃, CD₃OD, and DMSO-d₆. ¹³C-NMR spectra were recorded at 303.0 K on a 400 MHz or 500 MHz spectrometer using CDCl₃ (77.0 ppm), CD₃OD (49.15 ppm), or DMSO-d₆ (39.5 ppm) as reference. First-order chemical shifts and coupling constants (J/Hz) were obtained from one-dimensional spectra and assignments of proton resonance were based on 2D-COSY and 2D-NOESY. Dichloromethane (CH₂Cl₂; DCM), 1,2-dichloroethane, CH₃CN, and methanol were kept over 4 Å molecule sieves.

### Example 2

### Preparation of a 2′2-Difluoro-2′-Deoxyadanosine

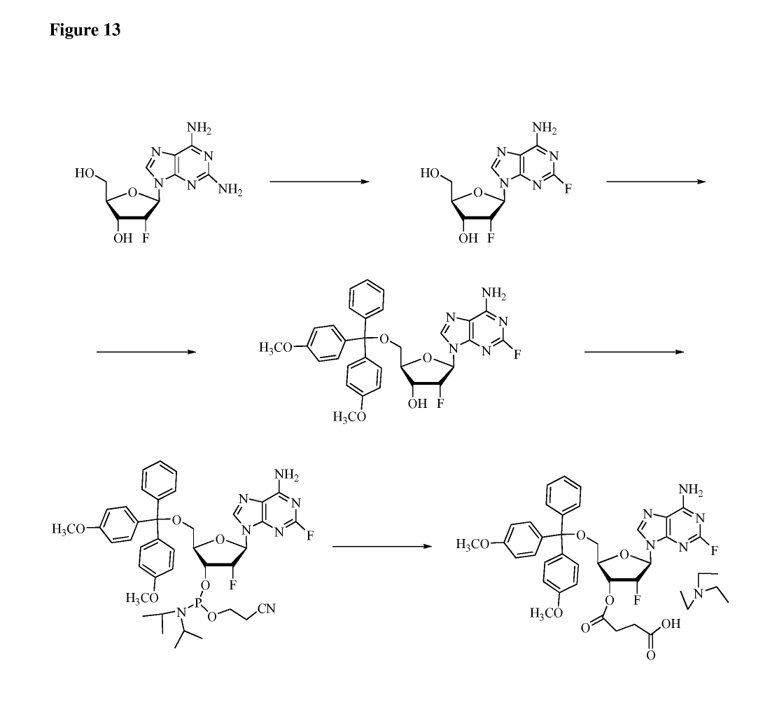

A reaction scheme showing one approach to the synthesis of 5′-O-(4,4′-dimethoxytrityl)-3′-O-succinyl-2,2′-difluoro-2′-deoxyadenosine is depicted in FIG. 13; synthetic procedures are provided below.

Preparation of 2,2′-Difluoro-2′-dexyadenosine. A 2 L polyethylene bottle was equipped with a magnetic stirrer, thermometer, dry ice/acetone bath and a stream of argon gas. Anhydrous pyridine (500 mL) was added and the solution was cooled to −20° C. To this was added 70% hydrogen fluoride in pyridine (400 mL). 2′-fluoro-2,6-diaminopurine riboside (90 g, 0.317 mol) was dissolved in the solution. Tert-butylnitrite (75 mL, 0.63 mol) was added in one portion and the reaction was stirred at 6-12° C. until the reaction was complete as judged by TLC (3 h). Sodium bicarbonate (2000 g) was suspended with manual stiffing in water (2 L) in a 20 L bucket. The reaction solution was slowly poured (to allow for evolution of carbon dioxide) into the aqueous layer with vigorous stiffing. The resulting solution was extracted with ethyl acetate (3×500 mL). The organic layers were combined and concentrated to a solid. The solid was mostly dissolved in methanol (300 mL) at reflux. The solution was cooled in a ice water bath and the resulting solid was collected, rinsed with methanol (2×50 mL) and dried under vacuum (1 mm Hg, 25° C., 24 h) to give 55 g of product as a dark gold solid. The mother liquor was concentrated and purified by column chromatography to give an additional 15.1 g of product for a total of 70.1 g (77%).

Preparation of 5′-O-(4,4′-Dimethoxytrityl)-2,2′-chfluoro-2′-deoxyadenosine. 2,2′-Di-fluoro-2′-deoxyadenosine (70 g, 0.244 mol) and dimethoxytrityl chloride (91.0 g, 0.268 mol) were dissolved in anhydrous pyridine (700 mL) and stirred at ambient temperature for 3 h. The reaction was quenched by the addition of methanol (50 mL) and then concentrated under reduced pressure to an oil. The residue was partitioned between ethyl acetate (1 L) and sat'd sodium bicarbonate (1 L mL). The aqueous layer was extracted with ethyl acetate (500 mL) once more and the combined extracts were concentrated under reduced pressure. The resulting solid was purified by crystallization from hexane-ethyl acetate (1:1) to give a light brown solid (120.1 g, 83%).

Preparation of [5′-O-(4,4′-Dimethoxytrityl)-2,2′-difluoro-2′-deoxyadenosin-3′-O-yl]-N,N-diisopropylaminocyanoethoxy phosphoramidite. 5′-O-(4,4′-Dimethoxytrityl)-2,2′-difluoro-2′-deoxyadenosine (90 g, 0.153 mol), 2-cyanoethyl tetraisopropylphosphorodiamidite (55.0 g, 0.183 mol), diisopropylamine tetrazolide (17.0 g, 0.1 mol) were dissolved in anhydrous dichloromethane (1 L) and allowed to stir at ambient temperature under an argon atmosphere for 16 h. The reaction was concentrated under reduced pressure to a thin oil and then directly applied to a silica gel column (200 g). The product was eluted with ethyl acetate-triethylamine (99:1). The appropriate fractions were combined, concentrated under reduced pressure, coevaporated with anhydrous acetonitrile and dried (1 mm Hg, 25° C., 24 h) to 109.1 g (90%) of light yellow foam.

Preparation of 5′-O-(4,4′-Dimethoxytrityl)-3′-O-succinyl-2,2′-difluoro-2′-deoxyadenosine. 5′-O-(4,4′-Dimethoxytrityl)-2,2′-difluoro-2′-deoxyadenosine (10 g, 16.9 mmol), dimethylaminopyridine (0.32 g, 2.6 mmol) and succinic anhydride (3.4 g, 34 mmol) were dissolved in anhydrous pyridine (50 mL) and stirred at ambient temperature under an argon atmosphere for 6 h. The reaction was quenched by the addition of water (20 mL) and then concentrated under reduced pressure to an oil. The oil was purified by column chromatography, eluted with methanol-dichloromethane-triethylamine (7:92:1). The appropriate fractions were collected and evaporated to give the product as a light yellow solid (10.4 g, 78%).

### Example 3

### Synthesis of N¹-(N-Methyl-3-Propionamide)-Pseudouridine oligonucleotide Building Blocks

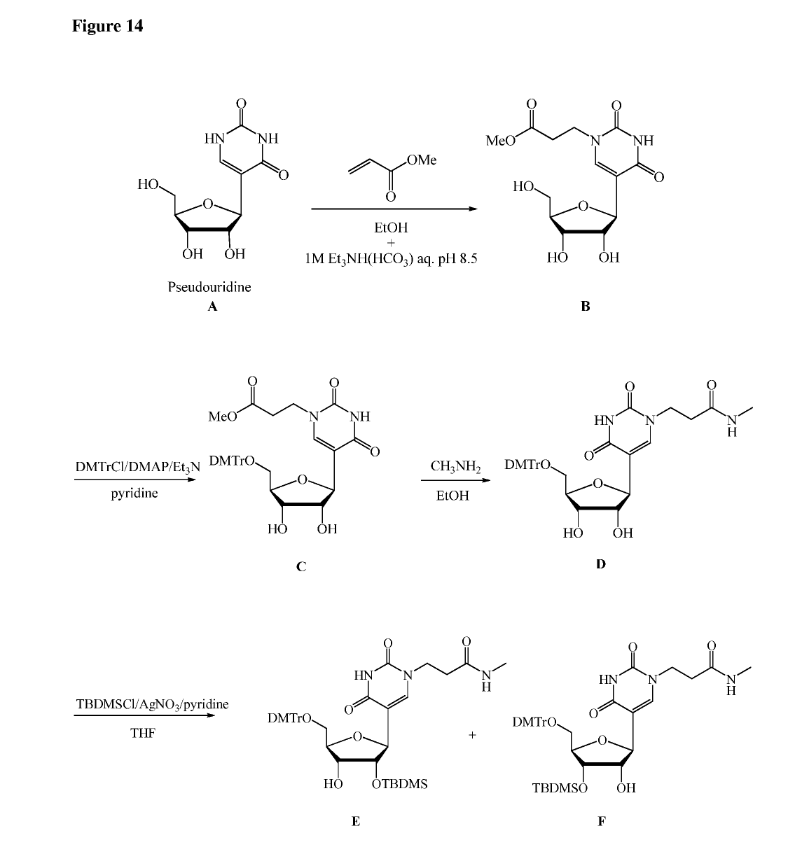

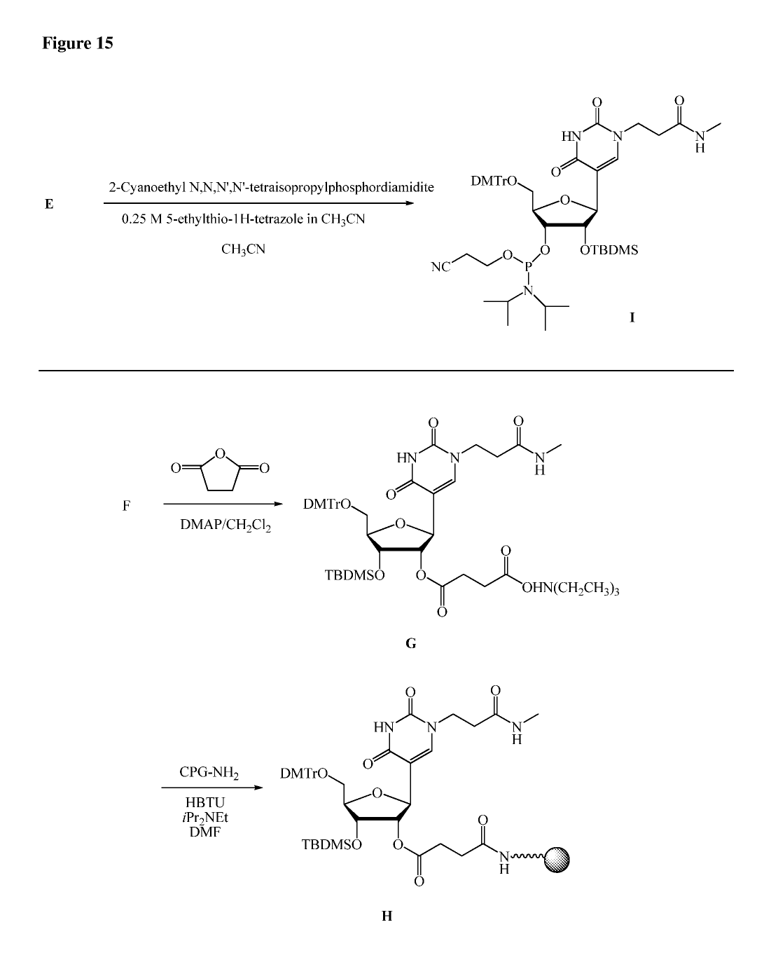

A reaction scheme showing one approach to the synthesis of N¹-(N-methyl-3-propionamide)-pseudouridine oligonucleotide building blocks is depicted in FIGS. 14 and 15; synthetic procedures and related spectral data are provided below.

For alkylation at N¹ position of pseudouridine, a reported method by Seela et al. for the 2′-deoxypseudouridine analog was used. Ramzaeva, N.; Rosemeyer, H.; Leonard, P.; Muhlegger, K.; Bergmann, F.; Von der Eltz, H.; Seela, F. Helvetica Chimica Acta 2000, 83, 1108-1126. To a solution of pseudouridine (20 g, 81.9 mmol) in 1M triethylammonium-bicarbonate buffer (pH 8.5, 780 mL) and EtOH (940 mL), methyl acrylate was dropwisely added. The reaction mixture was stirred overnight. After 16 hours, TLC showed a complete reaction. The solvent was removed and dried in vacuo to give a white foam. The crude material was purified by silica gel column chromatography (10% MeOH in CH₂Cl₂, R_{f}=0.23) to give B (26.6 g, 80.5 mmol, 98%). ¹H NMR (MeOH-d₄, 400 MHz) δ 7.77 (d, J=0.8 Hz, 1H), 4.58 (d, J=4.8 Hz, 1H), 4.15 (t, J=5.2 Hz, 1H), 4.05 (t, J=5.0 Hz, 1H), 3.98-4.02 (m, 2H), 3.91-3.94 (m, 1H), 3.80 (dd, J=12.0 Hz, 3.3 Hz, 1H), 3.67 (s, 3H), 3.66 (dd, J=12.0 Hz, 3.3 Hz, 1H), 2.73-2.77 (m, 2H). ¹³C NMR (CDCl₃, 100 MHz) δ 173.1, 165.4, 152.5, 145.8, 112.9, 85.6, 81.5, 75.6, 72.6, 63.3, 52.5, 46.2, 33.7. Molecular weight for C₁₃H₁₉N₂O₈ (M+H)⁺Calc. 330.11. Found 331.0.

To a solution of B (26.6 g, 80.54 mmol) in pyridine (250 mL), DMAP (1.97 g, 16.1 mmol, 0.2 eq.), DMTrCl (40.9 g, 120.8 mmol, 1.5 eq.), and Et₃N (16.8 mL, 120.8 mmol, 1.5 eq.) were added successively. The reaction mixture was stirred for 20 hours. The reaction was quenched by adding MeOH (10 mL), and evaporated all. The crude was extracted with CH₂Cl₂ and saturated NaHCO₃ aq., dried over anhydrous Na₂SO₄, and purified by silica gel column chromatography (5% MeOH in CH₂Cl₂ with 2% Et₃N, R_{f}=0.32) to give C (21.6 g, 34.1 mmol, 42%). ¹H NMR (MeOH-d₄, 400 MHz) δ 7.61 (d, J=1.2 Hz, 1H), 7.45-7.47 (m, 2H), 7.26-7.36 (m, 6H), 7.18-7.22 (m, 1H), 6.83-6.87 (m, 4H), 4.75 (dd, J=3.2 Hz, 1.2 Hz, 1H), 4.17 (dd, J=7.0 Hz, 5.1 Hz, 1H), 4.09 (dd, J=5.1 Hz, 3.2 Hz, 1H), 4.02-4.06 (m, 1H), 3.77 (s, 6H), 3.67-3.74 (m, 1H), 3.56 (s, 3H), 3.33-3.42 (m, 3H), 2.74 (q, J=7.2 Hz, 1H). ¹³C NMR (MeOH-d₄, 100 MHz) δ 172.7, 165.5, 160.2, 152.7, 146.4, 144.5, 137.4, 137.3, 131.5, 131.4, 129.6, 128.9, 128.0, 114.2, 114.0, 87.5, 83.0, 81.1, 76.2, 72.4, 64.7, 55.8, 52.4, 46.2, 33.5. Molecular weight for C₃₄H₃₆N₂NaO₁₀ (M+Na)⁺Calc. 655.23. Found 655.2.

A solution of methylamine in EtOH (33%) was added to compound C (9.98 g, 15.77 mmol) and the mixture was stirred at room temperature overnight. All solvent was removed and dried in vacuo to give compound D quantitatively (9.96 g, 15.77 mmol, R_{f}=0.45 developed with 10% MeOH in CH₂Cl₂). ¹H NMR (MeOH-d₄, 400 MHz) δ 7.52 (s, 1H), 7.43-7.45 (m, 2H), 7.15-7.33 (m, 7H), 6.82-6.84 (m, 4H), 4.68 (d, J=2.8 Hz, 1H), 4.09-4.15 (m, 2 H), 4.00-4.01 (m, 1H), 3.75-3.77 (m, 1H), 3.73 (s, 6H), 3.55-3.60 (m, 2H), 3.43-3.50 (m, 1H), 2.59 (s, 3H), 2.37 (t, J=6.4 Hz, 2H). ¹³C NMR (MeOH-d₄, 100 MHz) δ 172.8, 165.3, 160.2, 152.5, 146.5, 144.7, 137.4, 137.2, 131.5, 131.4, 129.5, 128.9, 127.9, 114.2, 113.7, 87.5, 83.3, 81.1, 75.9, 72.6, 64.8, 58.4, 55.8, 46.8, 35.4, 26.4, 18.4. Molecular weight for C₃₄H₃₇N₃NaO₉ (M+Na)⁺Calc. 654.24. Found 654.4.

Selective silylation at 2′-hydroxyl group was carried out according to Ogilvie's method. Hakimelahi, G. H.; Proba, Z. A.; Ogilvie, K. K. Tetrahedron Lett., 1981, 22, 4775-4778. To a solution of D (9.43 g, 14.93 mmol) in THF (166 mL), pyridine (4.47 mL, 55.24 mmol), and AgNO₃ (3.04 g, 17.92 mmol) was added. After 15 min, TBDMSCl (2.93 g, 19.41 mmol) was added and the reaction mixture was stirred for 24 hours under Ar gas. The reaction mixture was filtered through Celite, then extracted with CH₂Cl₂ and saturated NaHCO₃ aq., and dried over anhydrous Na₂SO₄. The crude was purified by silica gel column chromatography (EtOAc with 1% Et₃N) to give E (4.74 g, 6.35 mmol, 43%, R_{f}=0.36) and F (2.77 g, 3.71 mmol, 25%, R_{f}=0.20).

E: ¹H NMR (DMSO-d₆, 400 MHz) δ 11.3 (s, 1H), 7.77-7.78 (m, 1H), 7.41-7.45 (m, 3H), 7.28-7.33 (m, 6H), 7.19-7.23 (m, 1H), 6.87-6.89 (m, 4H), 4.60 (d, J=6.8 Hz, 1H), 4.53 (d, J=2.4 Hz, 1H), 4.04-4.06 (m, 1H), 3.88-3.92 (m, 1H), 3.76-3.81 (m, 1H), 3.73 (s, 6H), 3.58-3.62 (m, 2H), 3.24-3.29 (m, 1H), 3.12-3.15 (m, 1H), 2.31-2.34 (m, 2H), 0.87 (s, 9H), 0.066 (s, 3H), 0.043 (s, 3H). ¹³C NMR (DMSO-d₆, 100 MHz) δ 169.5, 162.5, 157.9, 150.3, 145.0, 143.2, 135.8, 135.6, 129.8, 129.7, 127.7, 126.5, 113.0, 111.0, 106.6, 85.2, 80.4, 80.0, 75.9, 70.5, 64.1, 63.4, 54.9, 44.9, 33.7, 30.1, 25.8, 25.7, 25.3, 20.6, 18.5, 18.0, 17.9, 13.5, −4.8, −4.9. Molecular weight for C₄₀H₅₁N₃NaO₉Si (M+Na)⁺Calc. 768.33. Found 768.4.

F: ¹H NMR (DMSO-d₆, 400 MHz) δ 11.3 (s, 1H), 7.79-7.82 (m, 1H), 7.51 (s, 1H), 7.40-7.43 (m, 2H), 7.19-7.32 (m, 7H), 6.87-6.89 (m, 4H), 4.75 (d, J=5.6 Hz, 1H), 4.50 (d, J=2.8 Hz, 1H), 3.90-3.96 (m, 2H), 3.84-3.88 (m, 1H), 3.72 (s, 6H), 3.67-3.70 (m, 2H), 3.04-3.17 (m, 2H), 2.38 (t, J=6.8 Hz, 2H), 0.73 (s, 9H), −0.036 (s, 3H), −0.11 (s, 3H). ¹³C NMR (DMSO-d₆, 100 MHz) δ 170.2, 169.6, 162.7, 158.0, 150.3, 148.9, 144.8, 143.8, 135.6, 135.5, 129.6, 127.7, 126.5, 113.0, 110.7, 106.6, 85.4, 80.9, 80.1, 73.1, 72.6, 63.9, 59.6, 54.9, 44.9, 33.6, 25.8, 25.6, 25.3, 20.7, 17.7, −4.65, −5.31.

To a solution of compound F (2.77 g, 3.71 mmol) in CH₂Cl₂ (100 mL), DMAP (1.36 g, 11.1 mmol) and succinic anhydride (742 mg, 7.42 mmol) were added. The reaction mixture was stirred overnight at room temperature. Silica gel column chromatography (7% MeOH in DCM with 7% Et₃N, R_{f}=0.23) of the crude mixture without aqueous work-up gave the compound G as the corresponding triethylammonium salt (3.5 g, 3.70 mmol, quantitatively).

¹H NMR (DMSO-d₆, 400 MHz) δ 11.40 (brs, 1H), 7.96-7.97 (m, 1H), 7.67 (s, 1H), 7.49-7.51 (m, 2H), 7.34-7.39 (m, 7H), 6.94-6.96 (m, 4H), 5.28 (dd, J=5.0 Hz, 3.4 Hz, 1H), 4.70 (d, J=3.2 Hz, 1H), 4.34 (dd, J=7.4 Hz, 5.4 Hz, 1H), 3.90-3.94 (m, 1H), 3.81-3.84 (m, 2 H), 3.80 (s, 6H), 3.28 (dd, J=10.4 Hz, 2.4 Hz, 1H), 3.09-3.19 (m, 1H), 2.56-2.66 (m, 6H), 2.37 (s, 3H), 0.76 (s, 9H), −0.021 (s, 3H), −0.105 (s, 3H). ¹³C NMR (DMSO-d₆, 100 MHz) δ 173.2, 171.0, 169.7, 162.3, 158.0, 150.3, 144.7, 137.2, 135.5, 135.4, 129.6, 127.7, 126.5, 125.2, 113.0, 109.5, 85.4, 81.0, 78.0, 74.7, 70.8, 63.6, 54.9, 51.9, 48.3, 45.1, 33.6, 28.9, 25.3, 20.9, 20.1, 17.4, 16.2, 10.8, 7.1, −5.2, −5.6. Molecular weight for C₄₄H₅₄N₃O₁₂Si (M−H)⁻ Calc. 844.35. Found 844.2.

To a solution of compound G (3.5 g, 3.70 mmol) in DMF (300 mL), HBTU (1.54 g, 4.06 mmol), iPr₂NEt (3.22 mL, 18.5 mmol), and CPG-NH₂ (Prime Synthesis CPG-500, NH₂ loading=140 mmol/g) (29 g, 4.06 mmol) were successively added. The mixture was shaken for 2 hours, then filtered, washed with CH₂Cl₂, and dried in vacuo. The residual amino groups were capped by shaking for 30 minutes with pyridine (225 mL), acetic anhydride (75 mL), and triethylamine (15 mL). After filtering, washing with CH₂Cl₂ (300 mL×2), then 50% MeOH/CH₂Cl₂ (300 mL), and drying in vacuo gave compound H (30.2 g). Loading: 75 mmol/g.

To a solution of compound E (4.55 g, 6.10 mmol) in acetonitrile (61 mL), 2-cyanoethyl N,N,N′,N′-tetraisopropylphosphordiamidite (2.33 mL, 7.32 mmol) and 0.25 M 5-ethylthio-1H-tetrazole in acetonitrile (29.28 mL, 7.32 mmol) were added at 0° C. The reaction mixture was stirred at room temperature for 2 hours. After removing the solvent, the crude material was purified by silica gel column chromatography (EtOAc with 1% Et₃N) to give I (5.50 g, 5.81 mmol, 95%, R_{f}=0.37 developed by EtOAc).

³¹P NMR (DMSO-d₆, 162 MHz) δ 147.98, 147.59. Molecular weight for C₄₉H₆₈N₅NaO₁₀PSi (M+Na)⁺Calc. 968.44. Found 968.3.

### Example 4

### Synthesis of N¹-(Cholesteryl-Alkyl)-Pseudouridine oligonucleotide Building Blocks

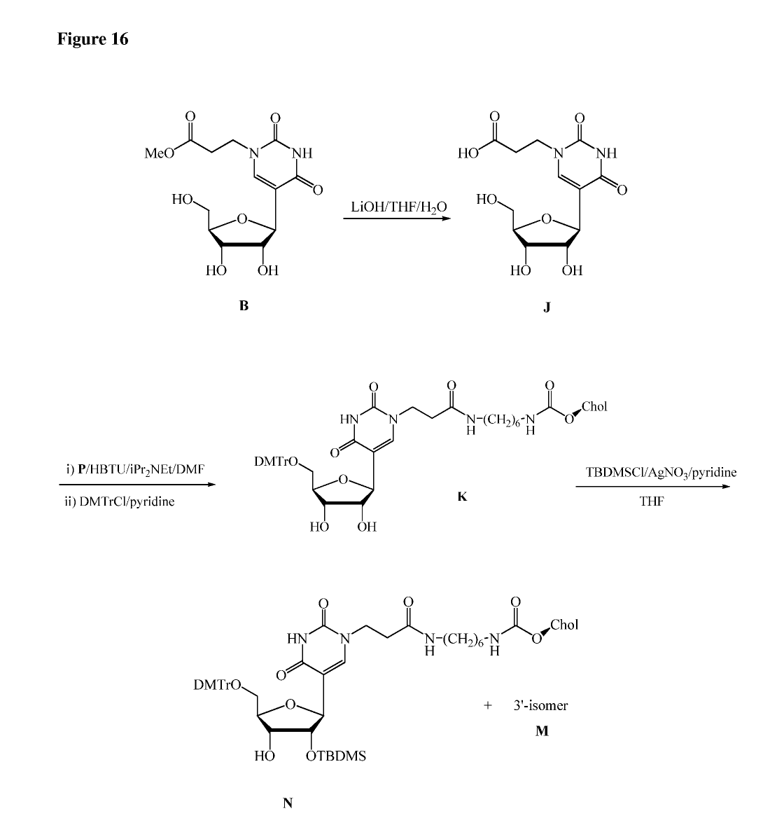

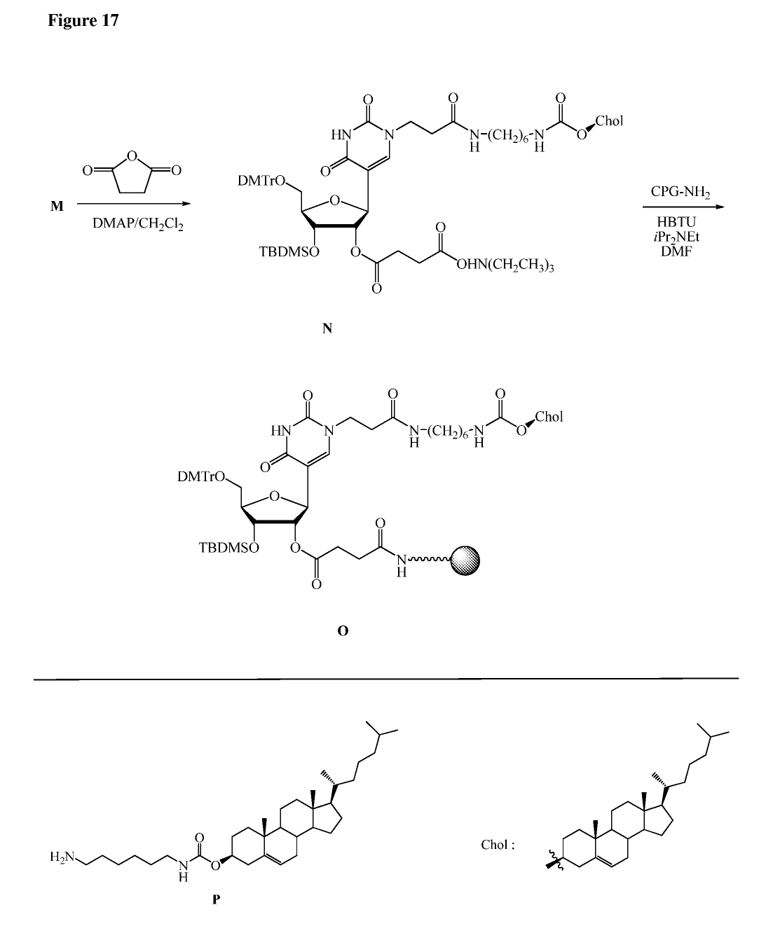

A reaction scheme showing one approach to the synthesis of N¹-(cholesteryl-alkyl)-pseudouridines is depicted in FIGS. 16 and 17; synthetic procedures and related spectral data are provided below.

To a solution of compound B (5.00 g, 15.1 mmol) in THF (100 mL) and H₂O (20 mL), lithium hydroxide monohydrate (1.03 g, 25.5 mmol) was added. The reaction mixture was stirred overnight. Additional lithium hydroxide monohydrate (500 mg, 11.9 mmol) was added. After 2 hours, the reaction mixture was treated with Amberlite IR-120 (plus) ion exchange resin. The resin was filtered off and washed with THF/H₂O. The filtrate was evaporated to give compound J as a white solid (4.78 g, quantitatively).

¹H NMR (DMSO-d₆, 400 MHz) δ 11.34 (s, 1H), 7.75 (s, 1H), 4.92-4.93 (m, 1H), 4.70-4.72 (m, 1H), 4.45 (d, J=4.0 Hz, 1H), 3.80-3.93 (m, 4H), 3.68-3.72 (m, 1H), 3.61 (dd, J=12.0 Hz, 3.2 Hz, 1H), 3.47 (dd, J=12.0 Hz, 4.0 Hz, 1H), 3.17 (d, J=3.2 Hz, 1H), 2.59 (t, J=7.0 Hz, 2H). ¹³C NMR (DMSO-d₆, 100 MHz) δ 172.1, 163.0, 150.4, 143.6, 111.4, 83.2, 79.0, 73.7, 70.4, 61.3, 44.1, 32.7. Molecular weight for C₁₂H₁₅N₂O₈ (M−H)⁻ Calc. 315.08. Found 315.1.

To a solution of compound J (4.78 g, 15.1 mmol), P (8.81 g, 16.65 mmol), iPr₂NEt (13.19 mL, 75.7 mmol) in DMF (320 mL) and CH₂Cl₂ (80 mL), HBTU (6.89 g, 18.17 mmol) was added. (For preparation of P see Ishi, T.; Iguchi, R.; Snip, E.; Ikeda, M.; Shinkai, S. Langmuir 2001, 17, 5825-5833.) The reaction mixture was stirred for 22 hours. TLC showed a coupled product (R_{f}=0.31 developed by 10% MeOH in CH₂Cl₂) and the solvent was evaporated in vacuo. To a solution of the crude in pyridine (100 mL), DMTrCl (5.90 g, 17.4 mmol) in pyridine (50 mL) was dropwisely added at 0° C. The reaction mixture was stirred overnight. After evaporation, the crude was washed with EtOAc and saturated NaHCO₃ aq., dried over anhydrous Na₂SO₄, and filtered. Silica gel column chromatography (5% MeOH in CH₂Cl₂ with 1% Et₃N, R_{f}=0.16) gave compound K (12.4 g, 10.98 mmol, 73%).

¹H NMR (DMSO-d₆, 400 MHz) δ 11.31 (s, 1H), 7.81 (t, J=5.5 Hz, 1H), 7.40-7.43 (m, 3H), 7.27-7.31 (m, 6H), 7.18-7.22 (m, 1H), 6.97 (t, J=5.7 Hz, 1H), 6.86-6.88 (m, 4H), 5.30-5.32 (m, 1H), 5.01 (d, J=4.7 Hz, 1H), 4.73 (J=6.7 Hz, 1H), 4.51 (d, J=2.8 Hz, 1H), 4.26-4.32 (m, 1H), 3.79-3.90 (m, 3H), 3.73 (s, 6H), 3.55-3.67 (m, 2H), 3.09-3.22 (m, 3H), 2.89-2.98 (m, 6H), 2.34 (t, J=6.4 Hz, 2H), 2.14-2.30 (m, 2H), 0.83-1.97 (m, 43H), 0.65 (s, 3 H).

Selective silylation at 2′-hydroxyl group was carried out according to Ogilvie's method.² To a solution of K (2.31 g, 2.05 mmol) in THF (23 mL), pyridine (0.613 mL, 7.59 mmol), and AgNO₃ (418 mg, 2.46 mmol) was added. After 15 min, TBDMSCl (402 mg, 2.67 mmol) was added and the reaction mixture was stirred overnight under Ar gas. Additional pyridine (0.184 mL, 2.28 mmol), AgNO₃ (125 mg, 0.736 mmol) and TBDMSCl (121 mg, 0.804 mmol) were added. The reaction mixture was filtered through Celite, then extracted with CH₂Cl₂ and saturated NaHCO₃ aq., and dried over anhydrous Na₂SO₄. The crude was purified by silica gel column chromatography (eluted with Hexane:EtOAc=1:1 to 1:8 with 1% Et₃N) to give L (620 mg, 0.499 mmol, 24%, R_{f}=0.74 developed by EtOAc) and M (440 mg, 0.354 mmol, 17%, R_{f}=0.53 developed by EtOAc).

L: ¹HNMR (DMSO-d₆, 400 MHz) δ 11.31 (brs, 1H), 7.81 (t, J=5.6 Hz, 1H), 7.40-7.45 (m, 3H), 7.28-7.33 (m, 6H), 7.21 (t, J=7.4 Hz, 1H), 6.97 (t, J=5.6 Hz, 1H), 6.87-6.89 (m, 4H), 5.31 (d, J=5.2 Hz, 1H), 4.57 (d, J=6.8 Hz, 1H), 4.53 (d, J=2.0 Hz, 1H), 4.29-4.32 (m, 1H), 4.04-4.05 (m, 1H), 3.88-3.92 (m, 1H), 3.79-3.83 (m, 1H), 3.73 (s, 6H), 3.51-3.63 (m, 2H), 3.23-3.27 (m, 2H), 3.12-3.15 (m, 1H), 2.90-2.96 (m, 6H), 2.18-2.34 (m, 4H), 0.83-1.98 (m, 52H), 0.65 (s, 3H), 0.069 (s, 3H), 0.044 (s, 3H). ¹³C NMR (DMSO-d₆, 100 MHz) δ 168.8, 162.5, 157.9, 155.5, 150.3, 144.9, 143.2, 139.7, 135.7, 135.5, 129.7, 127.7, 127.6, 126.5, 121.7, 113.0, 111.0, 85.2, 80.4, 79.9, 76.0, 72.6, 70.4, 63.9, 63.4, 59.6, 56.0, 55.5, 49.4, 45.0, 41.7, 38.3, 38.2, 36.5, 36.0, 35.6, 35.1, 33.8, 31.3, 31.2, 30.1, 29.3, 28.9, 27.8, 27.7, 27.3, 26.0, 25.9, 25.7, 23.8, 23.1, 22.6, 22.3, 20.6, 20.5, 18.9, 18.5, 18.4, 17.9, 14.0, 13.4, −4.8, −4.9. Molecular weight for C₇₃H₁₀₆N₄NaO₁₁Si (M+Na)⁺Calc. 1265.75. Found 1265.6.

M: ¹H NMR (DMSO-d₆, 400 MHz) δ 11.31 (s, 1H), 7.80-7.83 (m, 1H), 7.50 (s, 1 H), 7.40-7.42 (m, 2H), 7.19-7.31 (m, 7H), 6.97-7.00 (m, 1H), 6.86-6.88 (m, 4H), 5.31-5.32 (m, 1H), 4.70 (d, J=5.6 Hz, 1H), 4.50 (d, J=3.6 Hz, 1H), 4.26-4.32 (m, 1H), 3.85-3.99 (m, 2H), 3.72 (s, 6H), 3.65-3.69 (m, 3H), 3.08-3.18 (m, 2H), 2.89-2.97 (m, 7H), 2.16-2.40 (m, 4H), 0.83-1.99 (m, 43H), 0.73 (s, 9H), 0.65 (s, 3H), −0.036 (s, 3H), −0.12 (s, 3H). ¹³C NMR (DMSO-d₆, 100 MHz) δ 168.8, 162.5, 157.9, 155.5, 150.3, 144.9, 143.2, 139.7, 135.7, 135.5, 129.7, 127.7, 127.6, 126.5, 121.7, 113.0, 111.0, 85.2, 80.4, 79.9, 76.0, 72.6, 70.4, 63.9, 63.4, 59.6, 56.0, 55.5, 49.4, 45.0, 38.3, 38.2, 36.5, 36.0, 35.6, 35.1, 33.8, 31.3, 31.2, 30.1, 29.3, 28.9, 27.8, 27.7, 27.3, 26.0, 25.9, 25.6, 23.8, 23.1, 22.6, 22.3, 20.6, 20.5, 18.9, 18.5, 18.4, 17.9, 14.0, 13.4, 11.6, −4.8, −4.9. Molecular weight for C₇₃H₁₀₆N₄NaO₁₁Si (M+Na)⁺Calc. 1265.75. Found 1265.6.

To a solution of compound M (410 g, 0.330 mmol) in CH₂Cl₂ (30 mL), DMAP (121 mg, 0.989 mmol) and succinic anhydride (66 mg, 0.660 mmol) were added. The reaction mixture was stirred overnight at room temperature. Silica gel column chromatography (7% MeOH in DCM with 7% Et₃N, R_{f}=0.35) of the crude mixture without aqueous work-up gave the compound G as the corresponding triethylammonium salt (320 mg, 0.221 mmol, 67%).

¹H NMR (DMSO-d₆, 400 MHz) δ 11.40 (brs, 1H), 7.98 (t, J=5.2 Hz, 1H), 7.66 (s, 1H), 7.49 (d, J=7.6 Hz, 2H), 7.34-7.38 (m, 6H), 7.27 (t, J=7.2 Hz, 1H), 7.05 (t, J=5.4 Hz, 1 H), 6.93-6.95 (m, 4H), 5.38-5.39 (m, 1H), 5.26-5.28 (m, 1H), 4.70 (d, J=2.8 Hz, 1H), 4.31-4.39 (m, 2H), 3.90-3.94 (m, 3H), 3.79 (s, 6H), 2.96-3.06 (m, 6H), 2.57-2.69 (m, 5H), 2.23-2.37 (m, 2H), 1.83-2.04 (m, 5H), 0.90-1.58 (m, 42H), 0.75 (s, 9H), 0.72 (s, 3H), −0.027 (s, 3 H), −0.12 (s, 3H). ¹³C NMR (DMSO-d₆, 100 MHz) δ 173.2, 170.9, 169.0, 162.3, 158.0, 155.5, 150.3, 144.7, 144.6, 139.7, 135.5, 135.4, 129.7, 127.7, 127.6, 126.5, 121.7, 113.0, 109.5, 85.4, 80.9, 77.9, 74.9, 72.6, 70.7, 63.5, 62.5, 56.0, 55.5, 51.9, 49.4, 45.4, 41.7, 38.3, 36.5, 36.0, 35.6, 35.1, 33.7, 31.3, 31.2, 29.2, 28.8, 27.8, 27.7, 27.3, 26.0, 25.8, 25.3, 23.8, 23.1, 22.6, 22.3, 20.5, 18.9, 18.4, 17.4, 11.6, 10.1, 7.1, −5.2, −5.6. Molecular weight for C₇₇H₁₀₉N₄O₁₄Si (M−H)⁻ Calc. 1341.77. Found 1341.7.

To a solution of compound G (300 mg, 0.208 mmol) in DMF (20 mL), HBTU (87 mg, 0.229 mmol), iPr₂NEt (0.181 mL, 1.04 mmol), and CPG-NH₂ (Prime Synthesis CPG-500, NH₂ loading=140 mmol/g) (1.64 g, 0.229 mmol) were successively added. The mixture was shaken for 2 hours, then filtered, washed with CH₂Cl₂, and dried in vacuo. The residual amino groups were capped by shaking for 30 minutes with pyridine (15 mL), acetic anhydride (5 mL), and triethylamine (1 mL). After filtering, washing with CH₂Cl₂ (20 mL×2), then 50% MeOH/CH₂Cl₂ (20 mL), and drying in vacuo gave compound O (1.73 g). Loading: 68 μmmol/g.

### Example 5

### Synthesis of N¹-(2-Cyanoethyl)-Pseudouridines and N¹,N³-(2-Cyanoethyl)-Pseudouridines

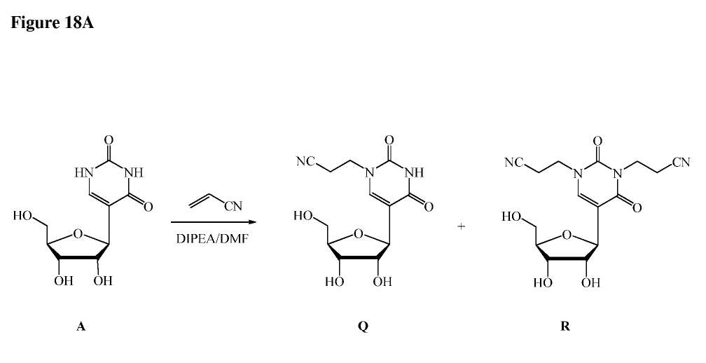

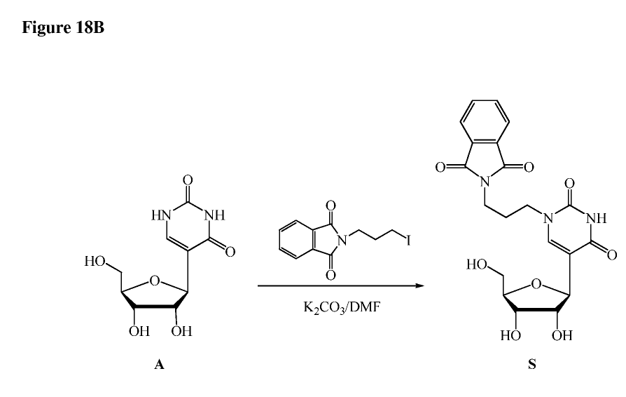

A reaction scheme showing one approach to the synthesis of N1- and N1,N3-(2-cyanoethyl)-pseudouridines is depicted in FIG. 18A; a synthetic procedure and related spectral data are provided below.

For alkylation at N1 and N3 position of pseudouridine with acrylonitrile, a reported procedure for the 2′-deoxypseudouridine analog was used. Cruickshank, K.; Ling, R. PCT Patent Application WO 03/066645 A2; hereby incorporated by reference. To a solution of A (100 mg, 0.410 mmol) in DMF (4 mL), acrylonitrile (0.180 mL, 2.77 mmol) and iPr₂NEt (0.179 mL, 1.03 mmol) was added. The reaction mixture was stirred at 45° C. for 24 hours. After removing the solvent, the crude material was purified by silica gel column chromatography (15% MeOH in CH₂Cl₂) to give compound Q (102 mg, 0.343 mmol, 84% R_{f}=0.24) and compound R (13 mg, 0.037 mmol, 9%, R_{f}=0.48).

Q: ¹H NMR (MeOD-d₄, 400 MHz) δ 7.78 (d, J=0.8 Hz, 1H), 4.61 (dd, J=4.8 Hz, 0.8 Hz, 1H), 4.13 (t, J=5.0 Hz, 1H), 3.95-4.10 (m, 3H), 3.90-3.93 (m, 1H), 3.80 (dd, J=12.2 Hz, 3.0 Hz, 1H), 3.66 (dd, J=12.2 Hz, 3.8 Hz, 1H), 2.88 (t, J=6.6 Hz, 2H). ¹³C NMR (MeOD-d₄, 100 MHz) δ 165.3, 152.3, 144.5, 118.6, 113.9, 85.4, 81.4, 75.7, 72.4, 63.1, 45.7, 17.8. Molecular weight for C₁₂H₁₆N₃O₆ (M+H)⁺Calc. 298.10. Found 298.1.

R: ¹H NMR (MeOD-d₄, 400 MHz) δ 7.85 (d, J=0.8 Hz, 1H), 4.68 (dd, J=4.4 Hz, 0.8 Hz, 1H), 4.22 (t, J=6.8 Hz, 2H), 4.02-4.15 (m, 4H), 3.90-3.93 (m, 1H), 3.83 (dd, J=12.0 Hz, 2.8 Hz, 1H), 3.69 (dd, J=12.0 Hz, 4.0 Hz, 1H), 2.89-2.92 (m, 2H), 2.83 (t, J=6.8 Hz, 2 H).

### Example 6

### Synthesis of N¹-(3-Phthalimido-Porpyl)-Pseudouridines

A reaction scheme showing one approach to the synthesis of N1-(3-phthalimido-porpyl)-pseudouridines is depicted in FIG. 18B; a synthetic procedure and related spectral data are provided below.

To a solution of A (50 mg, 0.205 mmol) in DMF (5 mL), N-(3-iodopropyl)phthalimide (71 mg, 0.226 mmol; Organic Syntheses, Collected Volumes 1998, 9, 502; and Organic Syntheses 1992, 70, 195) and K₂CO₃ (31 mg, 0.226 mmol) were added. The reaction mixture was stirred at room temperature overnight. After removing the solvent, the crude material was purified by silica gel column chromatography (15% MeOH in CH₂Cl₂) to give compound S (50 mg, 0.116 mmol, 57%, R_{f}=0.26). ¹H NMR (DMSO-d₆, 400 MHz) δ 11.33 (s, 1H), 7.83-7.89 (m, 4H), 7.80 (s, 1H), 4.92 (d, J=5.2 Hz, 1H), 4.73-4.76 (m, 1H), 4.68 (d, J=5.6 Hz, 1H), 4.43 (d, J=4.8 Hz, 1H), 3.97 (q, J=5.0 Hz, 1H), 3.87 (q, J=5.6 Hz, 1H), 3.57-3.76 (m, 5H), 3.42-3.48 (m, 1H), 1.91-1.94 (m, 2H). Molecular weight for C₂₀H₂₀N₃O₈ (M−H)⁻ Calc. 430.13. Found 430.0.

### Example 7

### Synthesis of Substituted-Pseudouridines

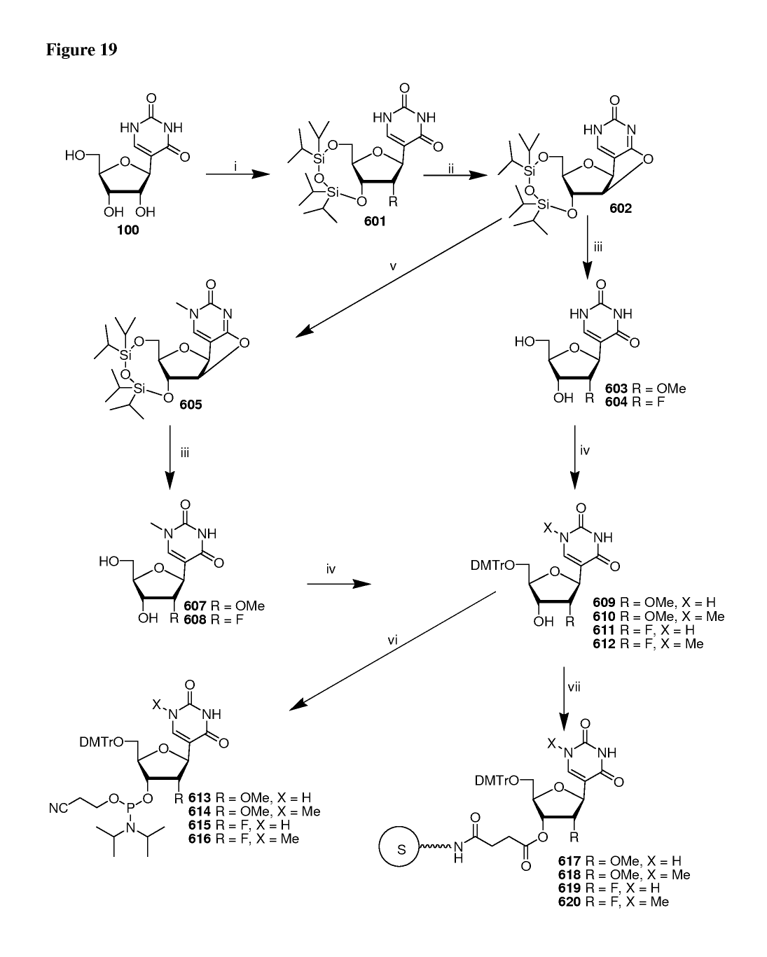

A reaction scheme showing approaches to the preparation of substituted-pseudouridines is shown in FIG. 19. Key: TIPDS-Cl₂, imidazoles/DMF; (ii) Tf₂O, DMAP/dichloromethane; (iii) HF-Py/dioxane, 125° C.; (iv) DMTr-C1, DMAP/pyridine; (v) a. Ac₂O, DMAP/pyridine and b. POCl₃, triazole, Et₃N/MeCN and aq. Ammonia and c. TES-Cl, pyridine then Bz-Cl; (vi) phosphitylation and (vii) succinic anhydride, DMAP followed by treatment with HBTU and diisopropylethylamine and amino linked solid support.

### Example 8

### Synthesis of 2′-Substituted N¹-Alkylated Pseudouridines

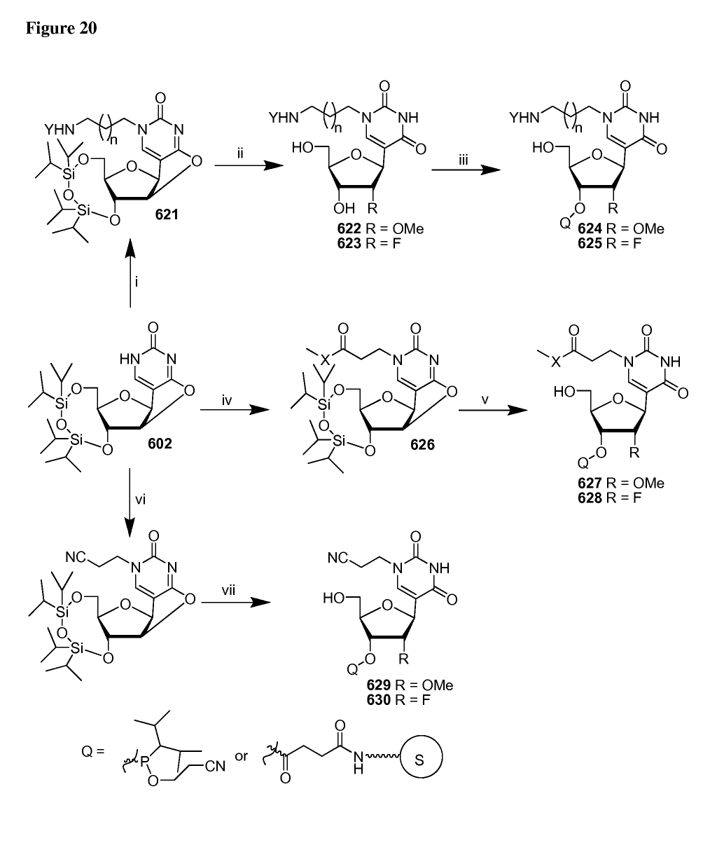

A reaction scheme showing approaches to the preparation of 2′-substituted alkylated pseudouridines is shown in FIG. 20. Key: TIPDS-C12, imidazoles/DMF; (ii) Tf20, DMAP/dichloromethane; (iii) HF-Py/dioxane, 125° C.; (iv) DMTr-C1, DMAP/pyridine; (v) a. Ac2O, DMAP/pyridine and b. POCl₃, triazole, Et₃N/MeCN and aq. Ammonia and c. TES-C1, pyridine then Bz-Cl; (vi) phosphitylation and (vii) succinic anhydride, DMAP followed by treatment with HBTU and diisopropylethylamine and amino linked solid support.

### Example 9

### Synthesis of 2′-Deoxy-2′-Fluoro-4′-Thio-5-Methyl Uracil and Cytosine

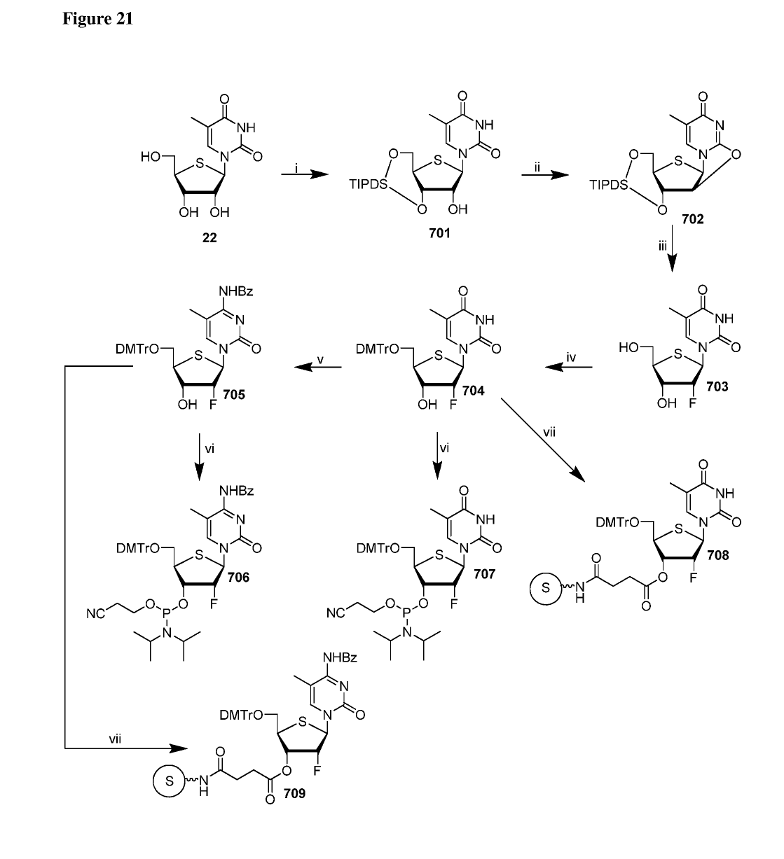

A reaction scheme showing approaches to the preparation of 2′-deoxy-2′-fluoro-4′-thio-5-methyl uracil and cytosine is shown in FIG. 21. Compound 22 is prepared as reported by Leydier et al. Leydier et al. Nucleosides & Nucleotides, 1995, 14, 1027. Compounds 704 and 705 are prepared from compound 22 according to the procedures reported by Takahashi et al. Takahashi et al. Tetrahedron, 2008, 64, 4313. Compounds 706, 707, 708 and 709 are prepared according to reported procedures. See Rajeev et al. Organic Lett. 2003, 5, 3005. Key: TIPDS-Cl₂, imidazoles/DMF; (ii) Tf₂O, DMAP/dichloromethane; (iii) HF-Py/dioxane, 125° C.; (iv) DMTr-Cl, DMAP/pyridine; (v) a. Ac₂O, DMAP/pyridine and b. POCl₃, triazole, Et₃N/MeCN and aq. Ammonia and c. TES-Cl, pyridine then Bz-Cl; (vi) phosphitylation and (vii) succinic anhydride, DMAP followed by treatment with HBTU and diisopropylethylamine and amino linked solid support

### Example 10

### Preparation of Phosphoramidite and Controlled Pore Glass Support of Selected Modified Nucleosides

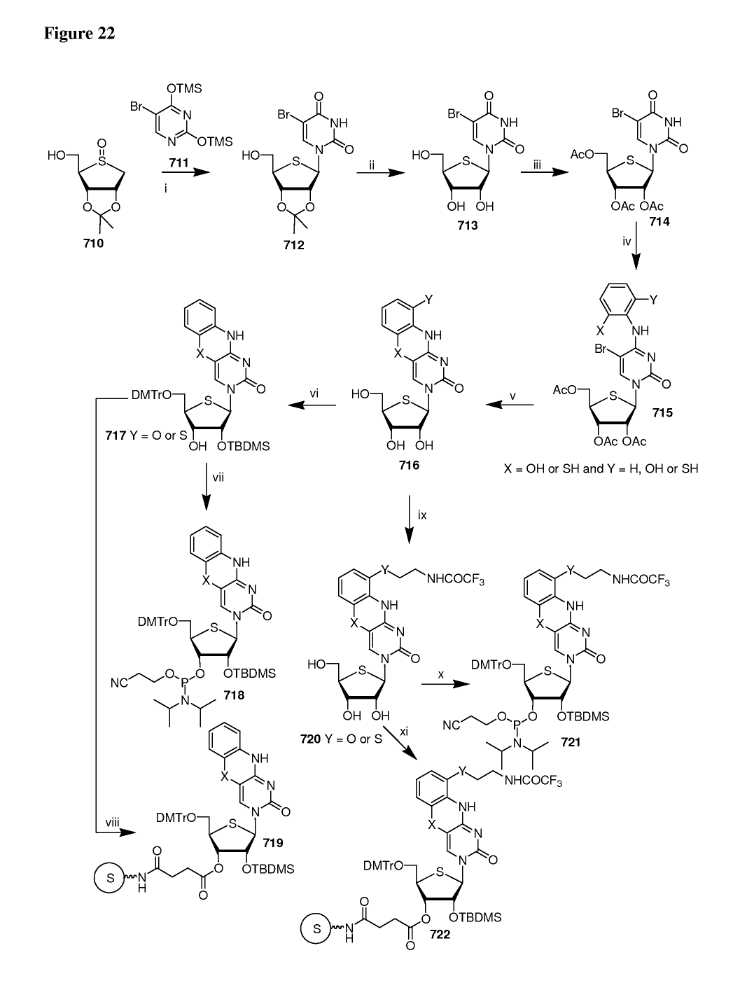

The preparation of selected compounds is depicted in FIGS. 22 to 24.

For FIG. 22: Compound 713 is prepared as reported in the literature (Yoshimura et al., Tetrahedron Lett., 2006, 47, 591). Acetylation of 713 yields 714. Compound 716 is obtained from 714 according to the procedures reported by Rajeev et al. (Organic Lett., 2002, 4, 4395). Phosphoramidites 718 and 721 and solid supports 719 and 722 are obtained according to reported procedures (Organic Lett., 2002, 4, 4395 and Organic Lett., 2003, 5, 3005). Key: (i) Compound 711, TMSOTf, iPr₂Net, dichloromethane, PhCH₃; (ii) Dowex 50W (H⁺ form), MeOH; (iii) Ac₂O, DMAP, pyridine; (iv) POCl₃, triazole, Et₃N then desired substituted aniline, Et₃N (V) CsF, EtOH, reflux; (vi) DMTr-Cl, DMAP, pyridine; (vii) phosphitylation and (vii) succinic anhydride, DMAP followed by treatment with HBTU and diisopropylethylamine and amino linked solid support.

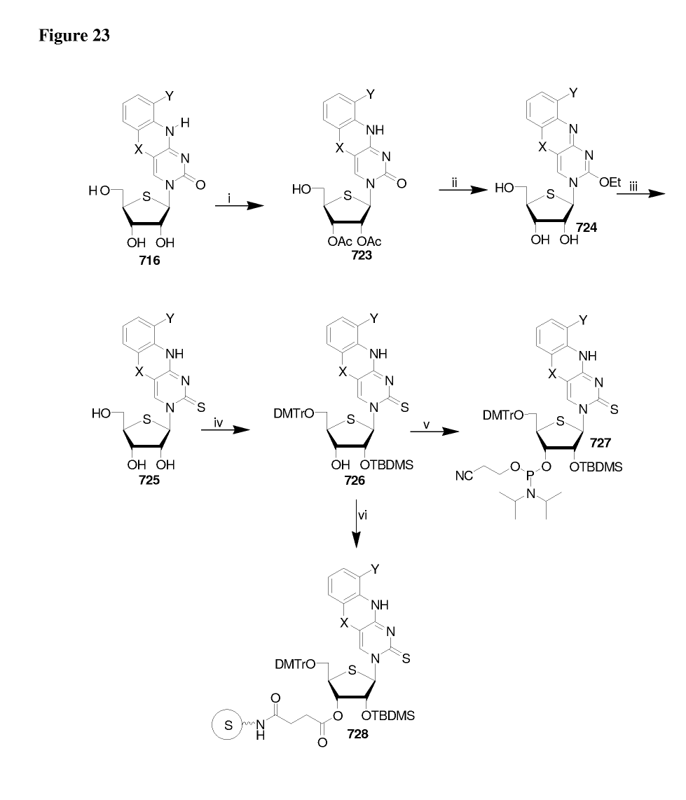

For FIG. 23: Compound 716 is obtained as described herein. Acetylation of compound 716 with acetic anhydride in pyridine in the presence of DMAP and subsequent removal of DMTr under mild acidic condition affords compound 723. Treatment of 723 with Ms-Cl in pyridine and subsequent reflux in anhydrous ethanol in the presence of NaHCO₃ affords compound 724. Reaction of compound 724 with sulfide in the presence of a strong base (TMG) yields compound 725 (Ref: Rajeev et al., Organic Lett., 2003, 5, 3005). The nucleic acid building blocks 727 and 728 are prepared from 725 as described in the previous examples. Key; (i) Ac₂O, DMAP, pyridine; (ii) a. Ts-Cl or Ms-Cl, pyridine and (b) EtOH, NaHCO₃, reflux; (iii) TMG, H₂S or Na₂S; (iv) a. DMTr-Cl, DMAP, pyridine and b. TBDMS-Cl, imidazole or TBDMS-C1, AgNO₃, pyridine, THF; (v) phosphitylation and (vi) succinic anhydride, DMAP followed by treatment with HBTU and diisopropylethylamine and amino linked solid support.

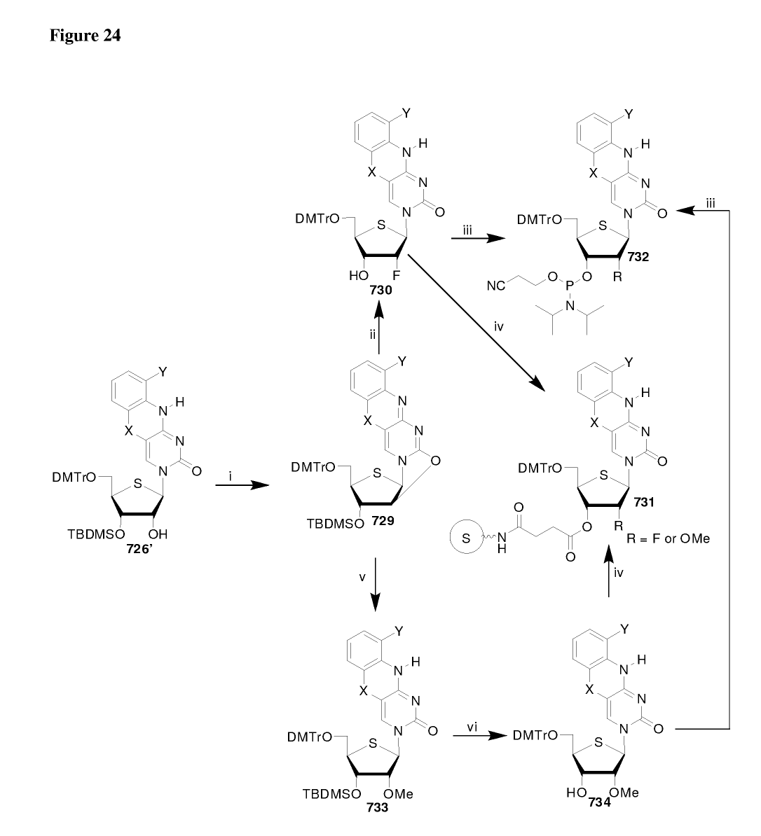

For FIG. 24: Compound 726′ is obtained similarly to as compound 726 described above. Key: (i) Tf₂O, DMAP/dichloromethane; (ii) HF-Py/dioxane, 125° C.; (iii) phosphitylation; (iv) succinic anhydride, DMAP followed by treatment with HBTU and diisopropylethylamine and amino linked solid support; (v) B(OMe)₃, MeOH, 150° C. and (vi) F.

### Example 11

### Synthesis of Phosphoramidite and Controlled Pore Glass Support of 5′-O-(4,4′-dimethoxitrityl)-2′-O-(tert-butyldimethylsilyl)-1-N-methylpseudo-uridine

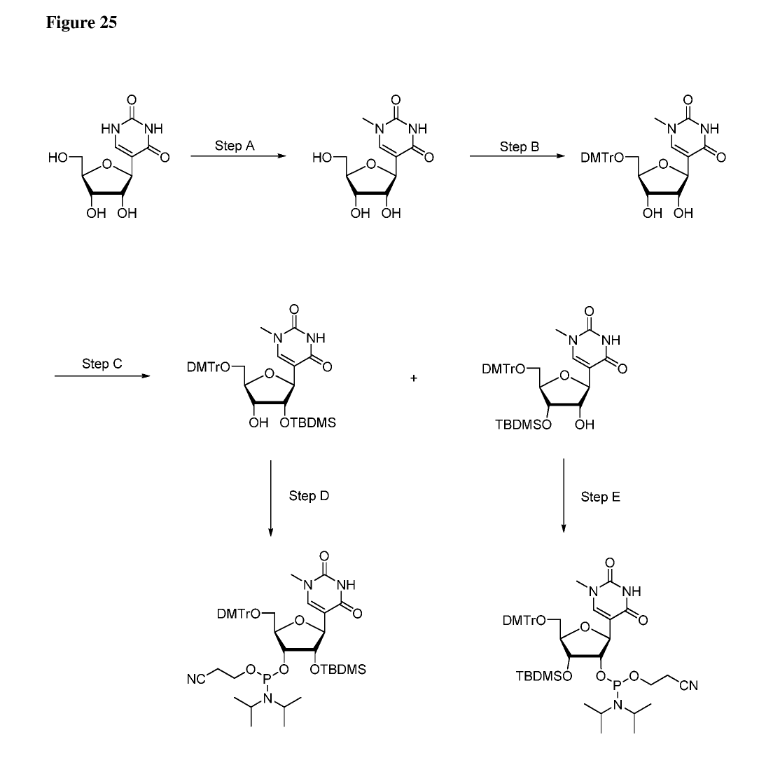

The synthesis of phosphoramidite and controlled pore glass support of 5′-O-(4,4′-dimethoxitrityl)-2′-O-(tert-butyldimethylsilyl)-1-N-methylpseudo-uridine is shown in FIG. 25. It is proposed that a similar approach may be taken with other compounds of the invention, and oligonucleotides comprising the same.

Step A: Preparation of 1-Methylpseudouridine. The title compound was prepared according to published procedure (Matsuda, A. et al., J. Org. Chem. 1981, 46, 3603-3609) and resulted in a pure compound (2.5 g, 84%) as an amorphous solid.

¹H-NMR (DMSO-d₆, 400 MHz): δ 11.33 (s, 1H, NH), 7.73 (s, 1H, H-6), 4.93 (d, 1 H, J=5.2 Hz, H-1′), 4.78 (dd, 1H, J=5.2, J=6.6 Hz), 4.73 (d, 1H, J=6.0 Hz), 4.44 (d, 1H, J=4.8 Hz), 3.92 (dd, 1H), 3.86 (dd, 1H), 3.70-3.67 (m, 1H), 3.62-3.57 (dq, 1H), 3.48-3.42 (m, 1H), 3.21 (s, 3H, CH₃).

Step B: Preparation of 5′-O-(4,4′-Dimethoxitrityl)-1-N-methylpseudouridine. 4,4′-Dimethoxtrityl chloride (437 mg, 1.29 mmol) was added to a solution of 1-N-methyl-pseudouridine (300 mg, 1.16 mmol) in dry pyridine (3 mL) in the presence of 4-N,N-dimethylaminopyridine (DMAP) (30 mg) and stirred at room temperature under an argon atmosphere overnight. The reaction mixture was concentrated to a crude residue and co-evaporated with dry toluene (3×10 mL). The crude residue was applied to a column of silica gel which was saturated with 2% triethylamine in hexanes, and eluted with ethyl acetate to give a pure title compound (540 mg, 78%) as an amorphous solid.

¹H-NMR (CDCl₃, 400 MHz): 6S 8.23 (d, 1H, J=6.4 Hz), 7.46 (s, 1H, ArH), 7.41-7.39 (m, 2H, ArH), 7.30-7.18 (m, 7H, ArH, H-6), 6.83-6.81 (m, 3H, ArH), 6.49 (d, 1H, ArH), 4.81 (d, 1H, J=6.0 Hz, H-1′), 4.29 (t, 1H, J=3.6, J=4.8 Hz), 4.19 (d, 1H), 4.15 (t, 1H, J=5.6, J=6.0 Hz), 3.79 (s, 6H, 2 OCH₃), 3.37 (dd, 1H, H-5″), 3.18 (br, 2H, 20H), 3.00 (s, 3H, CH₃).

Step C: Preparation of 5′-O-(4,4′-Dimethoxitrityl)-2′-O-(tert-butyldimethylsilyl)-1-N-methylpseudouridine. Anhydrous pyridine (3.64 mL) was added to a solution of 5′-O-(4,4′-dimethoxitrityl)-1-N-methylpseudouridine (2.67 g, 4.48 mmol) and AgNO₃ (934 mg, 5.69 mmol) in dry THF (32 mL) and stirred at room temperature for 20 min under an argon atmosphere. Followed by addition of tert-butyldimethylsilyl chloride (934 mg, 5.87 mmol) in dry THF (3 mL) and stirred at the same temperature for 3-4 h. The solids were filtered off and the filtrate was concentrated to a crude residue which was applied to a column of silica gel which was saturated with 2% triethylamine in hexanes, and eluted with hexane-ethyl acetate (1:1) to give a pure title compound (780 mg, 25%), and 5′-O-(4,4′-dimethoxitrityl)-3′-O-(tert-butyldimethylsilyl)-1-methylpseudouridine (800 mg, 25%) as amorphous solid.

Title compound: ¹H-NMR (CDCl₃, g-COSY, 400 MHz): δ 8.36 (d, 1H, J=11.6 Hz, NH), 7.63 (s, 1H, H-6), 7.46-7.42 (m, 2H, ArH), 7.36-7.22 (m, 7H, ArH), 6.88-6.80 (m, 4H, ArH), 4.86 (s, 1H, H-1′), 4.39-4.33 (m, 1H), 4.28-4.27 (m, 1H, H-2′), 3.97-3.95 (m, 1H), 3.79 (s, 6H, 2OCH₃), 3.53 (dd, 1H, H-5a′), 3.37 (dd, 1H, J=3.2, J=10.8 Hz, H-5b′), 2.74 (s, 3H, N—CH₃), 2.38 (d, 1H, J=9.6 Hz, 3′-OH), 0.93 (s, 9H, t-Bu), 0.28 (s, 3H, CH₃), 0.18 (s, 3H, CH₃).

5′-O-(4,4′-dimethoxitrityl)-3′-O-(tert-butyldimethylsilyl)-1-methylpseudouridine: ¹H-NMR (CDCl₃, g-COSY, 400 MHz): δ 8.30 (s, 1H, NH), 7.60 (s, 1H, H-6), 7.43-7.41 (m, 2 H, ArH), 7.32-7.24 (m, 7H, ArH), 6.85-6.82 (m, 4H, ArH), 4.91 (d, 1H, J=2.8 Hz, H-1′), 4.33 (dd, 1H, J=5.2, J=6.4 Hz, H-3′), 4.08-4.04 (dd, 1H, H-2′), 4.02-4.00 (dd, 1H, H-4′), 3.79 (s, 6H, 2OCH₃), 3.63 (dd, 1H, J=2.8, J=10.6 Hz, H-5″), 3.16 (dd, 1H, J=2.8, J=10.6 Hz, H-5b′), 2.89 (d, 1H, J=3.6 Hz, 2′-OH), 2.86 (s, 3H, N—CH₃), 0.81 (s, 9H, t-Bu), 0.028 (s, 3H, CH₃), −0.12 (s, 3H, CH₃).

Step D: Preparation of 5′-O-(4,4′-Dimethoxitrityl)-2′-O-(tert-butyldimethylsilyl)-1-methylpseudouridine-3′-O-caynoethyl-N,N-diisopropylphosphoramidate. 2-Cyanoethyl-N,N-diisopropylchlorophosphoramidite (450 mg, 1.92 mmol) was added to a solution of 5′-O-(4,4′-dimethoxitrityl)-2′-O-(tert-butyldimethylsilyl)-1-N-methylpseudouridine (680 mg, 0.96 mmol), diisopropylethylamine (910 μL, 5.28 mmol), and DMAP (34 mg) in dry dichloromethane (6-10 mL) and stirred at room temperature for 4-6 h under an argon atmosphere. The reaction mixture was concentrated to a crude residue which was applied to a column of silica gel which was saturated with 2% triethylamine in hexanes, and eluted with hexanes-ethyl acetate (2:1) to give a pure title compound (750 mg, 86%) as an amorphous solid.

³¹P-NMR (CDCl₃, 400 MHz): 147.90 (s), 145.58 (s).

Step E: Preparation of 5′-O-(4,4′-Dimethoxitrityl)-3′-O-(tert-butyldimethylsilyl)-1-N-methylpseudouridine-2′-O-caynoethyl-N,N-diisopropylphosphoramidate. 2-Cyanoethyl-N,N-diisopropylchlorophosphoramidite was added to a solution of 5′-O-(4,4′-dimethoxitrityl)-3′-O-(tert-butyldimethylsilyl)-1-N-methylpseudouridine (180 mg), diisopropyl-ethylamine, and DMAP in dry dichloromethane and stirred at room temperature for 4-6 h under argon atmosphere. The reaction mixture was concentrated to a crude residue which was applied to a column of silica gel which was saturated with 2% triethylamine in hexanes, and eluted with hexanes-ethyl acetate (2:1) to give a pure title compound as an amorphous solid.

³¹P-NMR (CDCl₃, 400 MHz): 148.90 (s), 146.58 (s).

### Example 12

### Oligonucleotide Synthesis

All oligonucleotides were synthesized on an AKTAoligopilot synthesizer or on an ABI 394 DNA/RNA synthesizer. Commercially available controlled pore glass solid supports (rU-CPG, 2′-O-methly modified rA-CPG and 2′-O-methyl modified rG-CPG from Prime Synthesis) or solid supported hydroxyprolinol-cholesterol-CPG were used for the synthesis. RNA phosphoramidites and 2′-O-methyl modified RNA phosphoramidites with standard protecting groups (5′-O-dimethoxytrityl-N-6-benzoyl-2′-t-butyldimethylsilyl-adenosine-3′-O—N,N′-diisopropyl-2-cyanoethylphosphoramidite, 5′-O-dimethoxytrityl-N⁴-acetyl-2′-t-butyldimethylsilyl-cytidine-3′-O—N,N′-diisopropyl-2-cyanoethylphosphoramidite, 5′-O-dimethoxytrityl-N²-isobutryl-2′-t-butyldimethylsilyl-guanosine-3′-O—N,N′-diisopropyl-2-cyanoethylphosphoramidite, 5′-O-dimethoxytrityl-2′-t-butyldimethylsilyl-uridine-3′-O—N,N′-diisopropyl-2-cyanoethylphosphoramidite, 5′-O-dimethoxytrityl-N-6-benzoyl-2′-O-methyl-adenosine-3′-O—N,N′-diisopropyl-2-cyanoethylphosphoramidite, 5′-O-dimethoxytrityl-N⁴-acetyl-2′-O-methyl-cytidine-3′-O—N,N′-diisopropyl-2-cyanoethylphosphoramidite, 5′-O-dimethoxytrityl-N²-isobutryl-2′-O-methyl-guano sine-3′-O—N,N′-diisopropyl-2-cyanoethylphosphoramidite, 5′-O-dimethoxytrityl-2′-O-methyl-uridine-3′-O—N,N′-diisopropyl-2-cyanoethylphosphoramidite and 5′-O-dimethoxytrityl-2′-deoxy-thymidine-3′-O—N,N′-diisopropyl-2-cyanoethylphosphoramidite) were obtained from Pierce Nucleic Acids Technologies and ChemGenes Research. The Quasar 570 phosphoramidite was obtained from Biosearch Technologies. The 5′-O-dimethoxytrityl-2′-t-butyldimethylsilyl-inosine-3′-O-N,N′-diisopropyl-2-cyanoethylphosphoramidite was obtained from ChemGenes Research. The 5′-β-dimethoxytrityl-2′-t-butyldimethylsilyl-(2,4)-difluorotolyl-3′-O-N,N′-diisopropyl-2-cyanoethylphosphoramidite (DFT-phosphoramidite) and the 5′-O-dimethoxytrityl-2′-t-butyldimethylsilyl-9-(2-aminoethoxy)-phenoxazin-1-yl-3′-O-N,N′-diisopropyl-2-cyanoethylphosphoramidite (G-clamp phosphoramidite) were synthesized in house.

3′-ligand conjugated strands were synthesized using solid support containing the corresponding ligand. For example, the introduction of cholesterol unit in the sequence was performed from a hydroxyprolinol-cholesterol phosphoramidite. Cholesterol was tethered to trans-4-hydroxyprolinol via a 6-aminohexanoate linkage to obtain a hydroxyprolinol-cholesterol moiety. 5′-end Cy-3 and Cy-5.5 (fluorophore) labeled siRNAs were synthesized from the corresponding Quasar-570 (Cy-3) phosphoramidite were purchased from Biosearch Technologies. Conjugation of ligands to 5′-end and or internal position is achieved by using appropriately protected ligand-phosphoramidite building block. An extended 15 min coupling of 0.1M solution of phosphoramidite in anhydrous CH₃CN in the presence of 5-(ethylthio)-1H-tetrazole activator to a solid bound oligonucleotide. Oxidation of the internucleotide phosphite to the phosphate was carried out using standard iodine-water as reported (1) or by treatment with tert-butyl hydroperoxide/acetonitrile/water (10:87:3) with 10 min oxidation wait time conjugated oligonucleotide. Phosphorothioate was introduced by the oxidation of phosphite to phosphorothioate by using a sulfur transfer reagent such as DDTT (purchased from AM Chemicals), PADS and or Beaucage reagent. The cholesterol phosphoramidite was synthesized in house, and used at a concentration of 0.1 M in dichloromethane. Coupling time for the cholesterol phosphoramidite was 16 minutes.

For the syntheses on AKTAoligopilot synthesizer, all phosphoramidites were used at a concentration of 0.2 M in CH₃CN except for guanosine and 2′-O-methyl-uridine, which were used at 0.2 M concentration in 10% THF/CH₃CN (v/v). Coupling/recycling time of 16 minutes was used for all phosphoramidite couplings. The activator was 5-ethyl-thio-tetrazole (0.75 M, American International Chemicals). For the PO-oxidation, 50 mM iodine in water/pyridine (10:90 v/v) was used and for the PS-oxidation 2% PADS (GL Synthesis) in 2,6-lutidine/CH₃CN (1:1 v/v) was used. For the syntheses on ABI 394 DNA/RNA synthesizer, all phosphoramidites, including DFT and G-clamp phosphoramidites were used at a concentration of 0.15 M in CH₃CN except for 2′-O-methyl-uridine, which was used at 0.15 M concentration in 10% THF/CH₃CN (v/v). Coupling time of 10 minutes was used for all phosphoramidite couplings. The activator was 5-ethyl-thio-tetrazole (0.25 M, Glen Research). For the PO-oxidation, 20 mM iodine in water/pyridine (Glen Research) was used and for the PS-oxidation 0.1M DDTT (AM Chemicals) in pyridine was used. Coupling of the Quasar 570 phosphoramidite was carried out on the ABI DNA/RNA synthesizer. The Quasar 570 phosphoramidite was used at a concentration of 0.1M in CH₃CN with a coupling time of 10 mins. The activator was 5-ethyl-thio-tetrazole (0.25 M, Glen Research) and 0.1M DDTT (AM Chemicals) in pyridine was used for PS oxidation.

Deprotection of oligonucleotides. For sequences synthesized on the AKTAoligopilot synthesizer, after completion of synthesis, the support was transferred to a 100 mL glass bottle (VWR). The oligonucleotide was cleaved from the support with simultaneous deprotection of base and phosphate groups with 40 mL of a 40% aq. methyl amine (Aldrich) 90 mins at 45° C. The bottle was cooled briefly on ice and then the methylamine was filtered into a new 500 mL bottle. The CPG was washed three times with 40 mL portions of DMSO. The mixture was then cooled on dry ice.

In order to remove the tert-butyldimethylsilyl (TBDMS) groups at the 2′ position, 60 mL triethylamine trihydrofluoride (Et₃N—HF) was added to the above mixture. The mixture was heated at 40° C. for 60 minutes. The reaction was then quenched with 220 mL of 50 mM sodium acetate (pH 5.5) and stored in the freezer until purification.

For sequences synthesized on the ABI DAN/RNA synthesizer, after completion of synthesis, the support was transferred to a 15 mL tube (VWR). The oligonucleotide was cleaved from the support with simultaneous deprotection of base and phosphate groups with 7 mL of a 40% aq. methyl amine (Aldrich) 15 mins at 65° C. The bottle was cooled briefly on ice and then the methylamine was filtered into a 100 mL bottle (VWR). The CPG was washed three times with 7 mL portions of DMSO. The mixture was then cooled on dry ice.

In order to remove the tert-butyldimethylsilyl (TBDMS) groups at the 2′ position, 10.5 mL triethylamine trihydrofluoride (Et₃N—HF) was added to the above mixture. The mixture was heated at 60° C. for 15 minutes. The reaction was then quenched with 38.5 mL of 50 mM sodium acetate (pH 5.5) and stored in the freezer until purification.

Quantitation of Crude Oligonucleotides. For all samples, a 10 μL aliquot was diluted with 990 μL of deionised nuclease free water (1.0 mL) and the absorbance reading at 260 nm was obtained.

Purification of Oligonucleotides. The unconjugated crude oligonucleotides were first analyzed by HPLC (Dionex PA 100). The buffers were 20 mM phosphate, pH 11 (buffer A); and 20 mM phosphate, 1.8 M NaBr, pH 11 (buffer B). The flow rate 1.0 mL/min and monitored wavelength was 260-280 nm. Injections of 5-15 μL were done for each sample.

The unconjugated samples were purified by HPLC on a TSK-Gel SuperQ-5PW (20) column packed in house (17.3×5 cm) or on a commercially available TSK-Gel SuperQ-5PW column (15×0.215 cm) available from TOSOH Bioscience. The buffers were 20 mM phosphate in 10% CH₃CN, pH 8.5 (buffer A) and 20 mM phosphate, 1.0 M NaBr in 10% CH₃CN, pH 8.5 (buffer B). The flow rate was 50.0 mL/min for the in house packed column and 10.0 ml/min for the commercially obtained column. Wavelengths of 260 and 294 nm were monitored. The fractions containing the full-length oligonucleotides were pooled together, evaporated, and reconstituted to about 100 mL with deionised water.

The ligand-conjugated crude oligonucleotides were first analyzed by LC/MS to determine purity. The ligand-conjugated sequences were HPLC purified on RPC-Source15 reverse-phase columns packed in house (17.3×5 cm or 15×2 cm). The buffers were 20 mM NaOAc in 10% CH₃CN (buffer A) and 20 mM NaOAc in 70% CH₃CN (buffer B). The flow rate was 50.0 mL/min for the 17.3×5 cm column and 12.0 ml/min for the 15×2 cm column. Wavelengths of 260 and 284 nm were monitored. The fractions containing the full-length oligonucleotides were pooled, evaporated, and reconstituted to 100 mL with deionised water.

Desalting of Purified Oligonucleotides. The purified oligonucleotides were desalted on either an AKTA Explorer or an AKTA Prime system (Amersham Biosciences) using a Sephadex G-25 column packed in house. First, the column was washed with water at a flow rate of 40 mL/min for 20-30 min. The sample was then applied in 40-60 mL fractions. The eluted salt-free fractions were combined, dried, and reconstituted in ˜50 mL of RNase free water.

Purity Analysis by Capillary Gel Electrophoresis (CGE), Ion-exchange HPLC (IEX), and Electrospray LC/Ms. Approximately 0.3 OD of each of the desalted oligonucleotides were diluted in water to 300 μL and were analyzed by CGE, ion exchange HPLC, and LC/MS.

Duplex Formation. For the fully double stranded duplexes, equal amounts, by weight, of two RNA strands were mixed together. The mixtures were frozen at −80° C. and dried under vacuum on a speed vac. Dried samples were then dissolved in 1×PBS to a final concentration of 40 mg/ml. The dissolved samples were heated to 95° C. for 5 min and slowly cooled to room temperature.

Tₘ Determination. For the partial double stranded duplexes and hairpins melting temperatures is determined. For the duplexes, equimolar amounts of the two single stranded RNAs are mixed together. The mixtures are frozen at −80° C. and dried under vacuum on a speed vac. Dried samples are then dissolved in 1×PBS to a final concentration of 2.5 μM. The dissolved samples are heated to 95° C. for 5 min and slowly cooled to room temperature. Denaturation curves are acquired between 10-90° C. at 260 nm with temperature ramp of 0.5° C./min using a Beckman spectrophotometer fitted with a 6-sample thermostated cell block. The Tₘ is then determined using the 1st derivative method of the manufacturer's supplied program.

TABLE 4
Synthesized oligonucleotides and RNAi agents

| Sequence | |
|---|---|
| (5′ → 3′) | Target |
| CUU ACG CUG AGU ACU UCG AdTdT | Luc |
| (SEQ ID NO: 1) | |
| U*CG AAG fUAC UCA GCG fUAA GdT*dT | |
| (SEQ ID NO: 2) | |
| C*UfU ACG CUG AGfU ACU UCG AdT*dT | Luc |
| (SEQ ID NO: 3) | |
| UCG AAG UAC UCA GCG UAA GdTdT | |
| (SEQ ID NO: 4) | |
| C*UfU ACG CUG AGfU ACU UCG AdT*dT | Luc |
| (SEQ ID NO: 5) | |
| U*CG AAG fUAC UCA GCG fUAA GdT*dT | |
| (SEQ ID NO: 6) | |
| CUU ACG CUG AGU ACU UCG AdTdT | Luc |
| (SEQ ID NO: 7) | |
| UCG AAG UAC UCA GCG UAA GdTdT | |
| (SEQ ID NO: 8) | |
| GGA UCA UCU CAA GUC UUA CdTdT | FVII |
| (SEQ ID NO: 9) | |
| GUA AGA CUU GAG AUG AUC CdTdT | |
| (SEQ ID NO: 10) | |
| GGA fUfCA fUfCfU fCAA GfUfC fUfCA fCdTsdT | |
| (SEQ ID NO: 11) | |
| GfUA AGA fCfUfU GAG AfUG AfUfC fCdT*dT | |
| (SEQ ID NO: 12) | |

### Example 13

### Serum Stability Assay for siRNA

A medium throughput assay for initial sequence-based stability selection was performed by the “stains all” approach. To perform the assay, an siRNA duplex was incubated in 90% human serum at 37° C. Samples of the reaction mix were quenched at various time points (at 0 min., 15, 30, 60, 120, and 240 min.) and subjected to electrophoretic analysis. Cleavage of the RNA over the time course provided information regarding the susceptibility of the siRNA duplex to serum nuclease degradation.

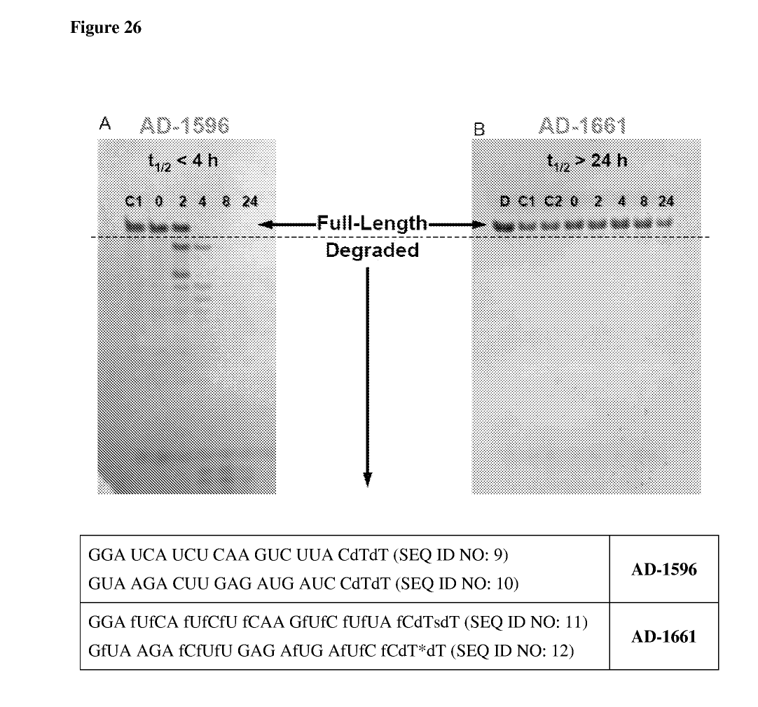

Results showed that a modified siRNA (AD-1661; FIG. 26B) has a half-life (t_{1/2}) greater than 24 hours when incubated in human serum at a temperature 37° C., as compared to an unmodified siRNA (AD-1596; FIG. 26A), which has a half-life less than 4 hours under the same incubation conditions, demonstrating that modified siRNA compositions described herein are more stable than unmodified siRNAs.

A radiolabeled dsRNA and serum stability assay was used to further characterize siRNA cleavage events. First, a siRNA duplex was 5′ end-labeled with ³²P on either the sense or antisense strand. The labeled siRNA duplex was incubated with 90% human serum at 37° C., and a sample of the solution was removed and quenched at increasing time points. The samples were analyzed by electrophoresis.

### Example 14

### Dual Luciferase Gene Silencing Assays

In vitro activity of siRNAs was determined using a high-throughput 96-well plate format luciferase silencing assay. Assays were performed in one of two possible formats. In the first format, HeLa SS6 cells were first transiently transfected with plasmids encoding firefly (target) and renilla (control) luciferase. DNA transfections were performed using Lipofectamine 2000 (Invitrogen) and the plasmids gWiz-Luc (Aldevron, Fargo, ND) (200 ng/well) and pRL-CMV (Promega, Madison, Wis.) (200 ng/well). After 2 h, the plasmid transfection medium was removed, and the firefly luciferase targeting siRNAs were added to the cells at various concentrations. In the second format, HeLa Dual-luc cells (stably expressing both firefly and renilla luciferase) were directly transfected with firefly luciferase targeting siRNAs. SiRNA transfections were performed using either TransIT-TKO (Mirus, Madison, Wis.) or Lipofectamine 2000 according to manufacturer protocols. After 24 h, cells were analyzed for both firefly and renilla luciferase expression using a plate luminometer (VICTOR², PerkinElmer, Boston, Mass.) and the Dual-Glo Luciferase Assay kit (Promega). Fireflylrenilla luciferase expression ratios were used to determine percent gene silencing relative to mock-treated (no siRNA) controls.

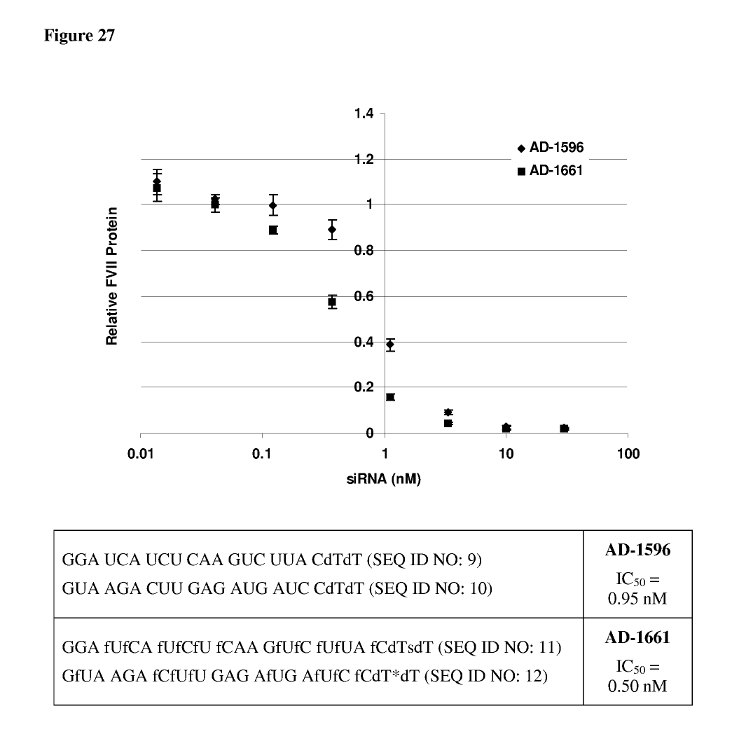

FIG. 27 shows that in HeLA SS6 cells stably transfected to express murine Factor VII (“FVII”), a 2′-fluoro modified siRNA (AD-1661) is approximately 2-fold more potent (IC₅₀ of 0.50 nM) in reducing Factor VII protein levels than an unmodified siRNA (AD-1596; IC₅₀ of 0.95 nM) having the same nucleotide sequence.

### Example 15

### Factor VII (FVII) In Vitro Assay

Cell Seeding for Transfection. Cells are seeded into 96-well plates one day prior to siRNA transfection at a density of 15,000 cells per well in media without antibiotics (150,000 cells/ml media, 100 μl per well).

Standard Transfection Conditions for FVII Stable Cell Line. Lipofectamine 2000 at a concentration of 0.5 μl/well is used for transfection in a 96 well plate set-up. Dilute FVII-targeting siRNA or control siRNA to a concentration of 6 nM in OptiMEM. Mix siRNA and transfection agent (lipofectamine 2000) and allow the complex to form by incubating 20 minutes at room temperature. After 20 minutes, add 50 μl of complexes (out of total 60 μl volume) to a single well containing cells that were seeded on the previous day (well already contains 100 μl of growth medium). Mix by gently pipetting up and down. Well now contains 150 μl total volume, 1 nM siRNA, 0.5 μl LF 2000 reagent. Return plate to 37 C. incubator. Remove media and replace with fresh media (1000/well) 24 hours after LF2000 complexes are added to the plate. 24 hours after media exchange, collect media supernatant for FVII activity assay. Levels of Factor VII protein in the supernatant may be determined in samples using a chromogenic assay (Coaset Factor VII, DiaPharma Group, OH or Biophen FVII, Aniara Corporation, OH) according to manufacturer protocols.

### Example 16

### FVII and apoB In Vivo Assay

In vivo rodent Factor VII and ApoB silencing experiments. C57BL/6 mice (Charles River Labs, MA) and Sprague-Dawley rats (Charles River Labs, MA) receive either saline or siRNA in desired formulations via tail vein injection at a volume of 0.01 mL/g. At various time points post-administration, animals are anesthesized by isofluorane inhalation and blood is collected into serum separator tubes by retroorbital bleed. Serum levels of Factor VII protein are determined in samples using a chromogenic assay (Coaset Factor VII, DiaPharma Group, OH or Biophen FVII, Aniara Corporation, OH) according to manufacturer protocols. A standard curve is generated using serum collected from saline treated animals. In experiments where liver mRNA levels are to be assessed, at various time points post-administration, animals are sacrificed and livers are harvested and snap frozen in liquid nitrogen. Frozen liver tissue is ground into powder. Tissue lysates are prepared and liver mRNA levels of Factor VII and apoB are determined using a branched DNA assay (QuantiGene Assay, Panomics, CA)

### Example 17

### Cytokine Induction in Human PBMC

A procedure for cytokine induction in human peripheral blood monouclear cells is as follows.
Isolation of peripheral blood mononuclear cells from human blood.
Direct incubation of oligonucleotides at 500 nM; or transfection at 130 nM with Lipofectamine-2000
ELISA with supernatants taken after 24 h.

### Example 18

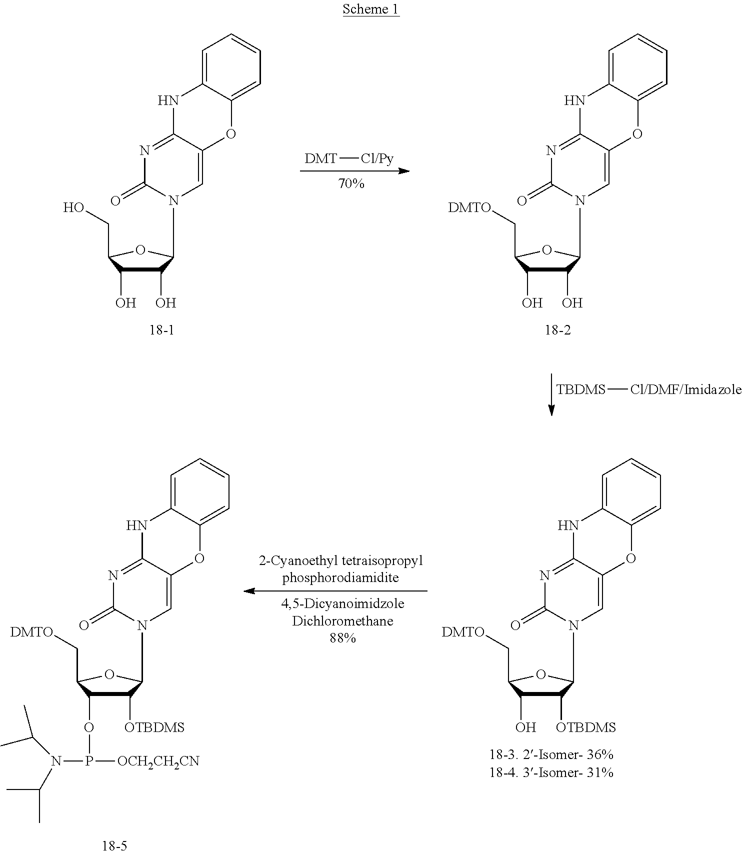

### Example 18.1

### Preparation of 3-(5′-O-Dimethoxytrityl-β-D-ribofuranosyl)-1,3-diaza-2-oxophenoxazine (18-2)

Phenoxazine nucleoside 18-1 (4.75 g, 14.25 mmol) (K-Y. Lin and M. Matteucci, J. Am. Chem. Soc. 1998, 120, 8531; S. C. Holmes, A. A. Arzumanov and M. J. Gait, Nucleic Acids Res., 2003, 31, 2759) was dried by coevaporation with dry pyridine (40 ml). Then it was suspended in dry pyridine (35 ml) and DMT-Cl (5.34 g, 15. 76 mmol) was added. After 3 h the reaction mixture was diluted with dichloromethane (200 ml) and washed with water. Organic layer was dried over sodium sulfate and evaporated. The residue was coevaporated with toluene and purified by silica gel column chromatography. Product was eluted using a gradient of 0-5% methanol in dichloromethane. The appropriate fractions containing the pure product were pooled and evaporated to give 6.38 g (70.4%) of compound 18-2.

¹H NMR (400 MHz, DMSO) δ 10.63 (s, 1H), 7.52-7.14 (m, 11H), 7.02-6.73 (m, 7H), 6.56-6.38 (m, 1H), 5.71 (d, J=4, 1H), 5.43 (d, J=8, 1H), 5.10 (d, J=8, 1H), 4.18-4.01 (m, 2H), 4.03-3.90 (m, 1H), 3.69, 3.70 (2 s, 6H), 3.32-3.28 (m, 1H), 3.23-3.10 (m, 1H).

### Example 18.2

### Silylation of 3-(5′-O-Dimethoxytrityl-β-D-ribofuranosyl)-1,3-diaza-2-oxophenoxazine (18-3, 18-4)

To a solution of 18-2 (5.88 g, 9.27 mmol) in dry DMF (30 ml) were added imidazole (1.63 g, 24 mmol) followed by TBDMS-Cl (1.81 g, 12 mmol). After stiffing the mixture at room temperature for 18 h, it was diluted with dichloromethane (150 ml) and washed with water (2×150 ml). Organic layer was dried over sodium sulfate and evaporated. The residue containing 2′-silylated and 3′-silylated products was chromatographed on a silica gel column. 2′-Isomer was eluted using a gradient of 10-25% ethyl acetate in dichloromethane. Finally 3′-isomer was eluted using 25-50% ethyl acetate in dichloromethane. The appropriate fractions containing pure products were collected and evaporated to give 2.5 g (36%) of 2′-isomer 18-3 and 2.1 g (30.2%) of 3′-isomer 18-4.

¹H NMR (400 MHz, DMSO) δ 10.65 (s, 1H), 7.94 (s, 1H), 7.59-7.14 (m, 10H), 7.01-6.68 (m, 6H), 6.44 (ddd, J=9.3, 7.9, 4.0, 1H), 5.69 (d, J=4, 1H), 5.07 (d, J=6.1, 1H), 4.29-4.15 (m, 1H), 4.11 (dd, J=11.1, 5.9, 1H), 4.05-3.94 (m, 1H), 3.70, 3.69 (2 s, 6H), 3.36 (dt, J=8.0, 4.1, 1H), 3.24-3.11 (m, 1H), 0.87 (s, 9H), 0.08, 0.09 2 s, 6H).

¹H NMR (400 MHz, DMSO) δ 10.64 (s, 1H), 7.51-7.11 (m, 10H), 6.99-6.71 (m, 6H), 6.56-6.41 (m, 1H), 5.69 (d, J=4, 1H), 5.22 (d, J=6.1, 1H), 4.13 (dd, J=13.8, 8.3, 1H), 4.10-3.92 (m, 1H), 3.94 (s, 1H), 3.67, 3.68 (2 s, 6H), 3.21 (ddd, J=15.3, 10.8, 3.7, 2H), 0.82-0.75 (s, 9H), 0.01, −0.08 (2 s, 6H).

### Example 18.3

### Preparation of 3-(5′-O-Dimethoxytrityl-2′-O-methyl-β-D-ribofuranosyl)-1,3-diaza-2-oxophenoxazine 3′-O-(2-cyanoethyl) N,N-diisopropylphosphoramidite(18-5)

To a solution of DMT Phenoxazine nucleoside (1.12 g, 1.5 mmol) in dry dichloromethane (5 ml) were added 4,5-dicyanoimidazole (159 mg, 1.35 mmol) followed by 2-cyanoethyl-N,N,N′,N″-tetraisopropylphosphorodiamidite (0.52 ml, 1.65 mmol) under an argon atmosphere. After stiffing for 4 h at room temperature the reaction mixture was diluted with ethyl acetate (50 ml) and washed with sodium acetate solution (0.5 M, 200 ml) followed by sat. sodium chloride solution (125 ml). Organic layer was dried over sodium sulfate and evaporated. The residue was purified by short silica gel column chromatography and the product was eluted using a mixture of dichloromethane and ethyl acetate containing triethylamine. The appropriate fractions containing the product were collected and evaporated to give 1.25 g (88%) of pure 18-5.

³¹P NMR (162 MHz, CD₃CN) δ 149.20, 148.86.

### Example 19

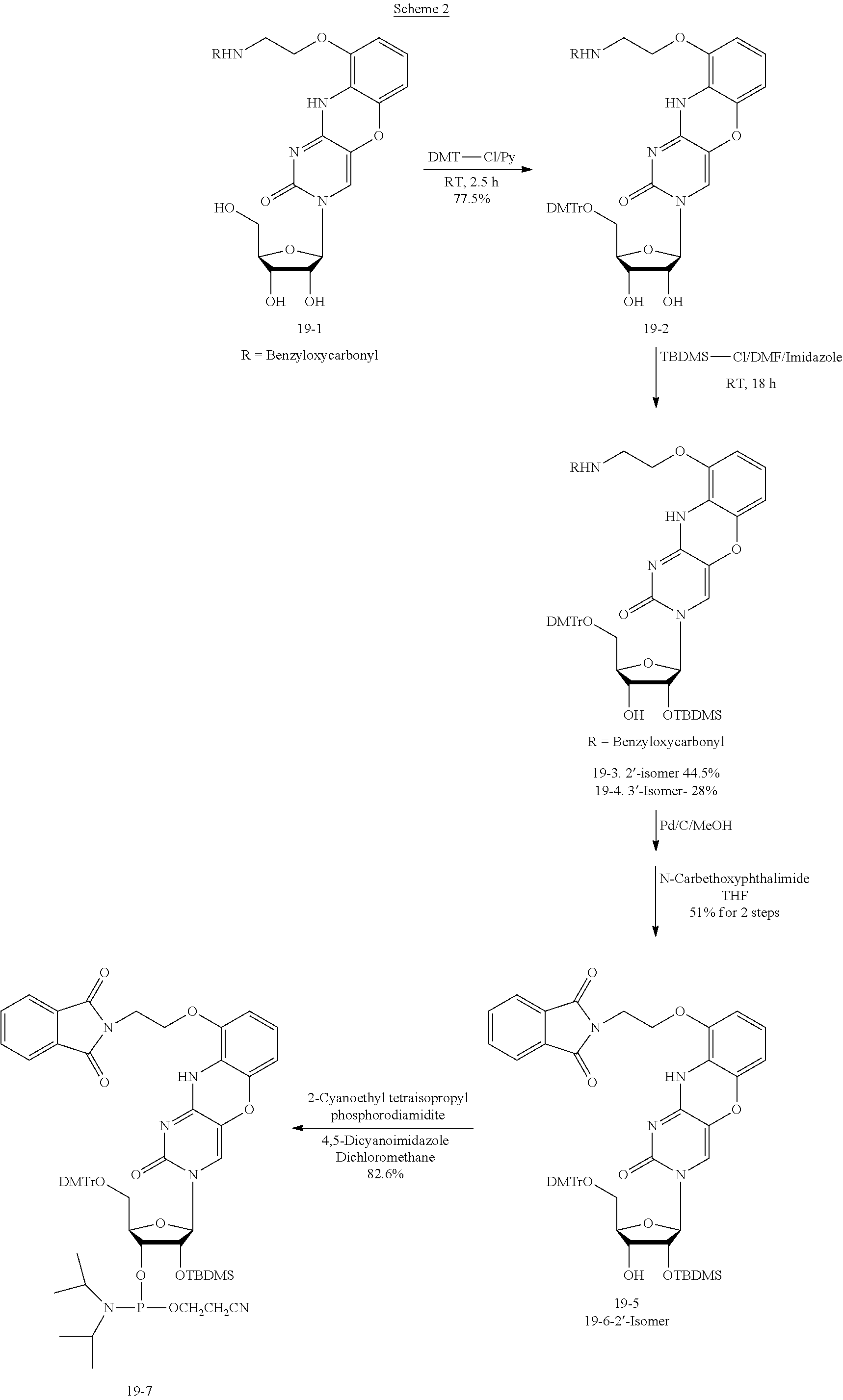

### Example 19.1

### Preparation of 3-(5′-O-Dimethoxytrityl-β-D-ribofuranosyl)-9-(2-N-benzylcarbamyl-ethoxy)-1,3-diaza-2-oxophenoxazine (19-2)

The sub (5 g, 9.5 mmol) was dried by coevaporation with dry pyridine (50 ml). Then it was dissolved in dry pyridine (35 ml) and DMT-Cl (3.8 g, 11.2 mmol) was added. After stirring at room temperature for 2.5 h, the reaction mixture was diluted with dichloromethane (150 ml) and washed with water (200 ml). Organic layer was dried over sodium sulfate and evaporated. The residue was coevaporated with toluene and purified by silica gel column chromatography. The product was eluted using a gradient of 0-5% methanol in dichloromethane. The fractions containing the pure product were pooled and evaporated to give 6.1 g (77.5%) of 19-2.

¹H NMR (400 MHz, DMSO) δ 9.84 (s, 1H), 7.76 (s, 1H), 7.56-7.14 (m, 16H), 7.00-6.82 (m, 4H), 6.83 (s, 1H), 6.59 (d, J=8.2, 1H), 6.13 (dd, J=8.2, 0.8, 1H), 5.72 (d, J=3.5, 1H), 5.44 (d, J=5.2, 1H), 5.10 (d, J=6.2, 1H), 5.04 (s, 2H), 4.20-4.01 (m, 2H), 4.03-3.87 (m, 2H), 3.69, 3.70 (2 s, 6H), 3.42 (d, J=4.9, 2H), 3.35-3.29 (m, 1H), 3.24-3.11 (m, 1H).

### Example 19.2

### Silylation of 3-(5′-O-Dimethoxytrityl-β-D-ribofuranosyl)-9-(2-N-benzylcarbamyl-ethoxy)-1,3-diaza-2-oxophenoxazine (19-3, 19-4)

To a solution of 19-2 (5.85 g, 7.05 mmol) in dry DMF (30 ml) were added imidazole (1.19 g, 17.5 mmol) followed by TBDMS-Cl (1.13 g, 7.5 mmol). After stiffing for 18 h, the reaction mixture was diluted with dichloromethane (150 ml) and washed with water (250 ml). Organic layer was dried over sodium sulfate and evaporated to give a foam containing 2′-silyl derivative, 3′-silyl derivative and bis silyl derivative. The mixture was chromatographed on a silica gel column and the pure products were eluted using a gradient of 5-60% ethyl acetate in dichloromethane. The pure fractions containing the product were evaporated to give 2.95 g (44.43%) of 2′-silyl derivate 19-3 and 1.86 g (28%) of 3′-silyl derivative 19-4.

¹H NMR ¹H NMR (400 MHz, DMSO) δ 9.77 (s, 1H), 7.80 (s, 1H), 7.55-7.07 (m, 16H), 6.81 (dd, J=8.9, 5.8, 4H), 6.70 (t, J=8.3, 1H), 6.51 (d, J=8.1, 1H), 6.01 (d, J=7.5, 1H), 5.63 (d, J=3.6, 1H), 5.03-4.94 (m, 3H), 4.21-3.99 (m, 2H), 3.99-3.89 (m, 2H), 3.85 (d, J=4.6, 2H), 3.61, 3.63 (2 s, 6H), 3.47-3.22 (m, 2H), 3.09 (d, J=9.1, 1H), 2.80 (s, 1H), 2.65 (d, J=0.4, 1H), 0.78 (s, 9H), 0.00, 0.01 (2 s, 6H).

¹H NMR (400 MHz, DMSO) δ 9.83 (s, 1H), 7.73 (s, 1H), 7.56-7.13 (m, 16H), 6.86 (dd, J=8.9, 1.5, 4H), 6.75 (t, J=8.3, 1H), 6.57 (d, J=8.3, 1H), 6.14 (d, J=8.2, 1H), 5.73 (d, J=3.8, 1H), 5.23 (d, J=6.1, 1H), 5.02 (s, 2H), 4.23-3.81 (m, 5H), 3.67, 3.68 (2 s, 6H), 3.39 (d, J=4.9, 2H), 3.28-3.11 (m, 2H), 1.96 (d, J=1.8, 2H), 0.75 (s, 9H), 0.00, −0.08 (2 s, 6H).

### Example 19.3

### 3-(5′-O-Dimethoxytrityl-2′-O-tert.butyldimethylsilyl-β-D-ribofuranosyl)-9-(2-N-phthalimido-ethoxy)-1,3-diaza-2-oxophenoxazine (19-5, 19-6)

To a solution of compound 19-3 (2.17 g) in dry methanol (60 ml) was added Pd/C (Degussa type, 240 mg, 10 wt % on carbon) and the mixture was stirred at room temperature under a positive pressure of hydrogen for 4 h. The reaction mixture was filtered and evaporated. The residue was dried in vaccuo for an hour and directly used in the next step.

To a solution of the compound obtained as above in dry THF (20 ml) were added N-carbethoxyphthalimide (0.52 g, 2.4 mmol) and 4-(dimethylamino)pyridine (0.293 g, 2.4 mmol). The mixture was stirred at room temperature overnight, diluted with dichloromethane (100 ml) and washed with water (50 ml). Organic layer was dried and evaporated. The residue containing 2′ and 3′ silyl isomers was chromatographed on a silica gel column and the pure isomers were eluted using a mixture of hexanes in ethyl acetate. The appropriate fractions containing the pure isomers were pooled and evaporated to give 1.1 g (51%) of 2′-isomer 19-5 and 0.6 g (27.8%) of 3′-isomer 19-6.

¹H NMR (400 MHz, DMSO) δ 9.28 (brs, 1H), 7.92-7.69 (m, 4H), 7.44-7.29 (m, 3H), 7.31-7.19 (m, 7H), 7.13 (t, J=7.3, 1H), 6.89-6.77 (m, 4H), 6.68 (t, J=8.3, 1H), 6.55 (d, J=8.0, 1H), 5.99 (dd, J=8.2, 1.0, 1H), 5.61 (d, J=3.7, 1H), 4.97 (d, J=6.1, 1H), 4.18-3.83 (m, 7H), 3.61, 3.63 (2 s, 6H), 3.34-3.25 (m, 1H), 3.09 (d, J=9.0, 1H), 0.79 (m, 9H), 0.00, 0.01 (2 s, 6H).

¹H NMR (400 MHz, DMSO) δ 7.97-7.74 (m, 4H), 7.48-7.09 (m, 11H), 6.86 (dd, J=9.0, 1.5, 4H), 6.74 (t, J=8.3, 1H), 6.61 (d, J=7.8, 1H), 6.13 (dd, J=8.2, 1.0, 1H), 5.70 (d, J=4.1, 1H), 5.22 (d, J=6.1, 1H), 4.20 (t, J=6.0, 2H), 4.12 (t, J=5.4, 1H), 3.97 (ddt, J=21.1, 14.3, 5.9, 4H), 3.67, 3.68 (2 s, 6H), 3.28-3.11 (m, 2H), 0.85 (s, 1H), 0.75 (s, 9H), 0.00, −0.06 (2 s, 6H).

### Example 19.4

### 3-(5′-O-Dimethoxytrityl-2′-O-tert.butyldimethylsilyl-β-D-ribofuranosyl)-9-(2-N-phthalimido-ethoxy)-1,3-diaza-2-oxophenoxazine 3′-O-(2-cyanoethyl) N, N,-diisopropylphosphoramidite (19-7)

To a solution of the nucleoside 19-5 (1 g, 1.06 mmol) in dry dichloromethane (10 ml) were added 3,4-dicyanoimidazole (0.112 g, 0.95 mmol) followed by 2-cyanoethyl-N,N,N′,N′-tetraisopropylphosphorodiamidite (0.35 ml, 1.12 mmol) under an argon atmosphere. After stirring for 4 h an additional 0.12 ml of phosphitylating agent was added. Thirty minutes later the reaction mixture was diluted with ethyl acetate (75 ml) and washed with saturated sodium bicarbonate solution (150 ml). Organic layer was dried over sodium sulfate and evaporated. The residue was purified on a silica gel column. The product was eluted using a mixture of dichloromethane and ethyl acetate containing triethylamine. The fractions containing the pure product were pooled and evaporated to give 1 g (82.6%)

³¹P NMR (162 MHz, CD₃CN) δ 149.42, 148.77.

### Example 20

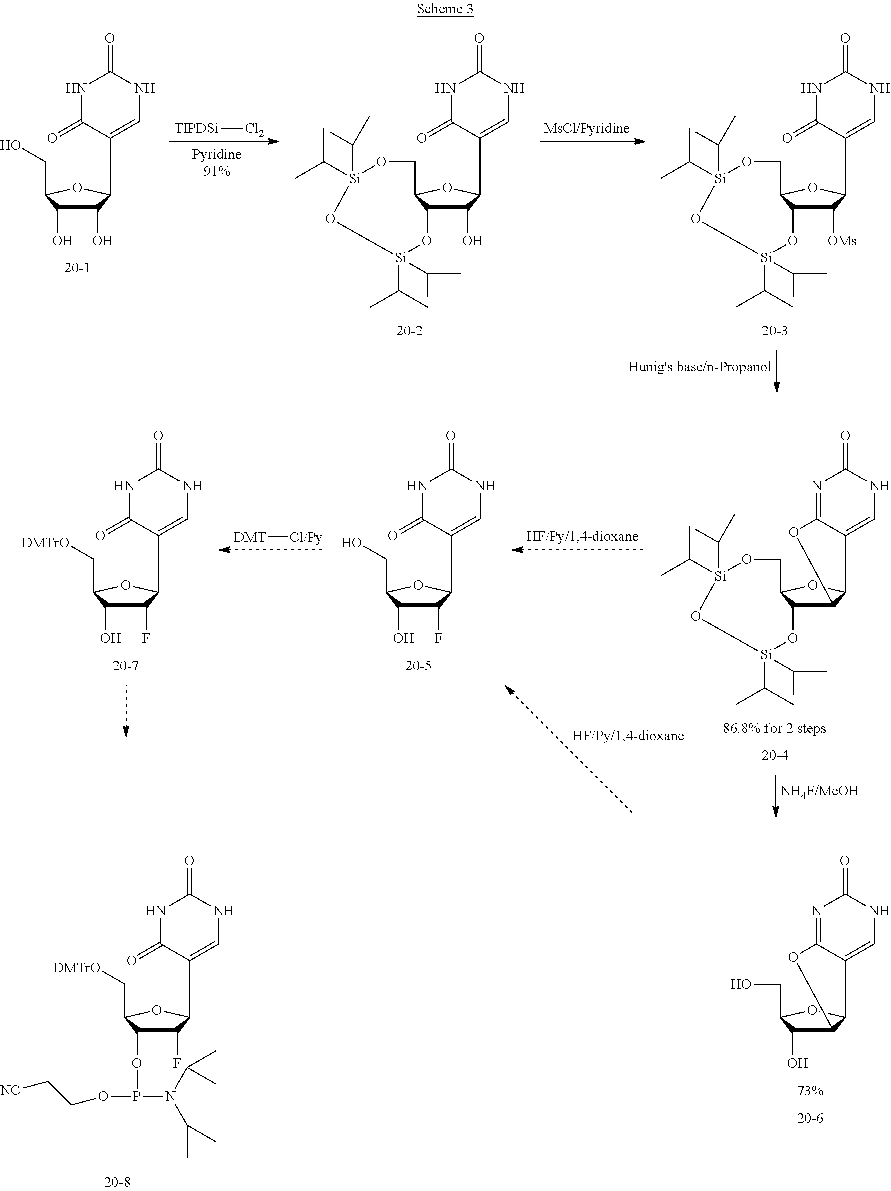

### Example 20.1

### Preparation of 3′,5′-O-(1,1,3,3-Tetraisopropyl-1,3-disiloxanediyl)-pseudouridine (20-2)

Pseudouridine 20-1 (20 g, 82 mmol) was dried by coevaporation with dry pyridine (80 ml). The dried material was suspended in dry pyridine (150 ml) and the reaction flask was cooled in an ice bath. To this cold suspension 1,3-dichloro-1,1,3,3-tetraisopropyldisiloxane (30 g, 95 mmol) was added in about 5 min. The reaction flask was allowed to warm to room temperature slowly by leaving the flask in the ice bath. After stirring for 18 h the reaction mixture was diluted with dichloromethane (200 ml) and washed with 10% aqueous sodium bicarbonate solution (150 ml). Aq layer was extracted with dichloromethane (50 ml) and the combined organic layer was dried over sodium sulfate and evaporated. The residue was coevaporated with toluene and purified by silica gel column chromatography. The product was eluted using a gradient of 0-4% methanol in dichloromethane and the appropriate fractions containing the product were evaporated to give 39 g (97.8%) of a colorless foam.

### Example 20.2

### Preparation of 2′,4-Anhydro-3′,5′-O-(1,1,3,3-tetraisopropyl-1,3-disiloxanediyl)-pseudouridine (20-4)

The substrate 20-2 (38.29 g, 78.7 mmol) was dried by coevaporation with dry pyridine (80 ml). Then it was dissolved in dry pyridine (80 ml) and the reaction flask was cooled in an ice bath. To this cold solution, methanesulfonyl chloride (8 ml, mmol) was added in approximately 3 min. The resulting mixture was stirred for 18 h while allowing the reaction mixture to warm slowly to room temperature by leaving the reaction flask in the ice bath. The reaction was quenched by the addition of 3 ml of sat. sodium bicarbonate solution. After 30 min the reaction mixture was diluted with dichloromethane (200 ml) and washed with 10% sodium bicarbonate solution (150 ml). Organic layer was dried over sodium sulfate and evaporated. The residue was coevaporated with toluene and used in the next step without any further purification.

The product obtained as above was suspended in a mixture of n-propanol (300 ml) and N,N-diisopropylethyl amine (75 ml) and the mixture was heated at 120 C. for 2 h. The clear solution obtained was allowed to cool to room temperature and kept at room temperature overnight. The crystallized product was collected by filtration after washing with a small amount of n-propanol it was dried at 40 C. in vaccuo to give 32 g (86.8%) of pure 20-4.

¹H NMR (400 MHz, DMSO) δ 7.98 (s, 1H), 5.36 (d, J=6.7, 1H), 5.27 (dd, J=6.7, 4.2, 1H), 4.25 (dd, J=8.8, 4.2, 1H), 3.88 (qd, J=13.0, 3.2, 2H), 3.70 (dt, J=8.8, 3.1, 1H), 1.18-0.86 (m, 28H).

### Example 20.3

### Preparation of 2′,4-Anhydropseudouridine (20-6)

Silyl anhydro derivative 20-4 (20.5 g, 43.74 mmol) was suspended in methanol (250 ml) to which ammonium fluoride (6 g) was added and heated under reflux for 1 h. Solvent was evaporated and the product was purified by a short silica gel column chromatography. Product was eluted using a gradient of 0-20% methanol in dichloromethane. The fractions containing the product were collected and evaporated to give 7.23 g (73%) of 20-6.

¹H NMR (400 MHz, DMSO) δ 11.30 (s, 1H), 7.91 (s, 1H), 5.66 (d, J=5.1, 1H), 5.29 (d, J=6.0, 1H), 5.04 (dd, J=6.0, 2.1, 1H), 4.70 (t, J=5.6, 1H), 4.06 (td, J=5.4, 2.1, 1H), 3.65 (dt, J=5.7, 4.6, 1H), 3.44-3.13 (m, 2H).

### Example 20.4

### 5′-O-Dimethoxytrityl-2′-fluoropseudouridine 3′-O-(2-cyanoethyl)N, N-diisopropylphosphoramidite (20-8)

Compound 20-4 or 20-6 on treatment with HF/Pyridine in anhydrous dioxane at elevated temperature gives 2′-fluoropseudouridine 20-5 which can be converted to the corresponding 5′-dimethoxytrityl derivative 20-7, by treating with 1. 25 equivalents 4,4′-dimethoxytrityl chloride in anhydrous pyridine. Compound 20-7 (1 mmol) on treatment with 2-cyanoethyl-N,N,N′,N″-tetraisopropylphosphorodiamidite (1.2 mmol) in dichloromethane in the presence of 3,4-dicyanoimidazole (0.9 mmol), work up of the reaction followed by silica gel column chromatography gives the corresponding 3′-phposphoramidite 20-8. Alternatively this compound can also be prepared by treating 20-7 with 2-cyanoethyl-N,N-diisopropylchloro phosphoramidite in dichloromethane in the presence of Hunig's base.

### Example 21

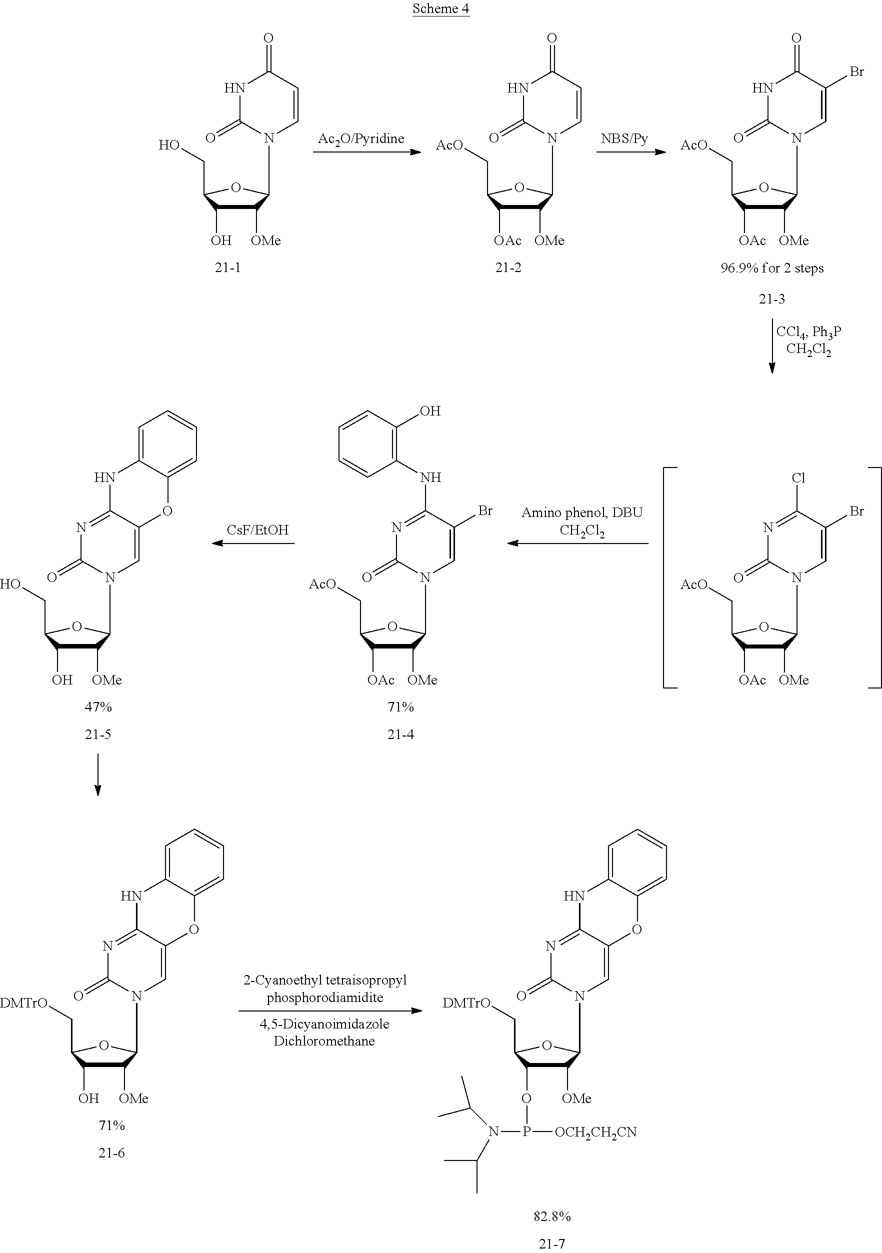

### Example 21.1

### Preparation of 5-Bromo-3′,5′-diacetyl-2′-O-methyluridine (21-3)

To a solution of 2′-O-methyluridine (25 g, 96.9 mmol) in dry pyridine (200 ml) was added acetic anhydride (50 ml, mmol) and the reaction mixture was stirred at room temperature for 18 h. The reaction was quenched by the addition of methanol (50 ml). After 30 min the solvent was evaporated. The residue was dissolved in dichloromethane (350 ml) and washed with saturated sodium bicarbonate solution (4×150 ml). Aqueous layer was extracted with dichloromethane (2×75 ml) and the combined organic layers was dried over sodium sulfate and evaporated. The residue was coevaporated with toluene and used directly in the next reaction without any further purification.

To a solution of the compound obtained as above in dry pyridine (250 ml) was added N-bromosuccinimide (26.7 g, mmol) and mixture was stirred at room temperature for 20 h. The reaction mixture was diluted with dichloromethane (450 ml) and washed with saturated sodium bicarbonate solution (3×250 ml). Aqueous layer was extracted with dichloromethane (2×100 ml) and the combined organic layer was dried over sodium sulfate and evaporated. The residue was coevaporated with toluene and the product was purified by silica gel column chromatography. The product was eluted using a gradient of 0-2% methanol in dichloromethane. Appropriate fractions containing the pure product were pooled and evaporated to 39.5 g (96.9%) of 21-3.

¹H NMR (400 MHz, DMSO) δ 11.95 (s, 1H), 8.11 (s, 1H), 5.78 (d, J=4.9, 1H), 5.17 (t, J=5.2, 1H), 4.36-4.15 (m, 4H), 3.30 (d, J=5.7, 4H), 2.08 (d, J=2.4, 6H).

### Example 21.2

### Preparation of 5-Bromo-3′,5′-diacetyl 2′-O-methyl-N⁴-(2-hydroxyphenyl)cytidine (21-4)

To a solution of 21-3 (13.3 g, 31.66 mmol) in dry dichloromethane (100 ml) and carbon tetrachloride (100 ml) was added triphenylphosphine (12.46 g, 47.5 mmol). The mixture was heated under reflux for 3 h. An additional 5.4 g of triphenylphosphine was added and the reaction continued overnight. The reaction mixture was allowed to cool to room temperature and diluted with 80 ml of dichloromethane. To this 2-aminophenol (8.73 g, 80 mmol) followed by DBU (11.96 ml, 80 mmol) were added and the mixture was stirred at room temperature overnight. The reaction mixture was diluted with dichloromethane (50 ml) and washed with 5% citric acid (450 ml) followed by water (100 ml). Organic layer was dried over sodium sulfate and evaporated. The residue was triturated with toluene followed by dichloromethane to give a solid which was collected by filtration. The solid was washed with a small amount of dichloromethane. The filtrate was directly loaded on a silica gel column and the product was eluted using a gradient of 02-% methanol in dichloromethane. The fractions containing the pure product were collected and evaporated to give a total yield of 11.5 g (71%) of 21-4.

¹H NMR (400 MHz, DMSO) δ 10.20 (s, 1H), 8.45 (s, 1H), 8.16 (s, 1H), 8.08 (dd, J=8.0, 1.3, 1H), 7.20-6.71 (m, 3H), 5.85 (d, J=4.4, 1H), 5.15 (t, J=5.4, 1H), 4.38-4.13 (m, 4H), 3.32 (d, J=2.0, 6H), 2.09 (d, J=8.5, 6H).

### Example 21.3

### Preparation of 3-(2′-O-Methyl-β-D-ribofuranosyl)-1,3-diaza-2-oxophenoxazine (21-5)

To a suspension of 21-4 (3.5 g) in absolute ethanol (120 ml) was added cesium fluoride (10.2 g) and the mixture was heated under reflux for 24 h. The solvent was evaporated and the residue was purified by silica gel column chromatography. The product was eluted using a gradient of 0-10% methanol in dichloromethane in dichloromethane. The appropriate fractions containing the pure product were pooled and evaporated to give 1.2 g (47.2%) of 21-5.

¹H NMR (400 MHz, DMSO) δ 10.35 (d, J=35.4, 1H), 9.97 (d, J=11.1, OH), 8.36 (d, J=7.5, OH), 7.46 (d, J=29.6, 1H), 6.68-6.45 (m, 4H), 5.55 (d, J=4.2, 1H), 4.98 (t, J=4.8, 1H), 4.82 (d, J=6.1, 1H), 3.83 (d, J=5.6, 1H), 3.60-3.52 (m, 1H), 3.50-3.38 (m, 2H), 3.32 (dd, J=4.5, 2.7, 1H), 3.07 (s, 6H).

### Example 21.4

### Preparation of 3-(5′-O-Dimethoxytrityl-2′-O-methyl-β-D-ribofuranosyl)-1,3-diaza-2-oxophenoxazine (21-6)

To a solution of the nucleoside (2.3 g) in dry pyridine (40 ml) was added DMT-Cl (2.56 g) and the mixture was stirred at room temperature for 3.5 h. The reaction mixture was diluted with dichloromethane (150 ml) and washed with water (100 ml). Organic layer was dried over sodium sulfate and evaporated. The residue was coevaporated with toluene and purified by silica gel column chromatography. The product was eluted using a gradient of 0-4% methanol in dichloromethane. Appropriate fractions containing the pure product were pooled and evaporated to give 3.05 g (71%) of 21-6.

¹H NMR (400 MHz, DMSO) δ 10.65 (s, 1H), 7.60-7.18 (m, 10H), 7.03-6.75 (m, 6H), 6.47-6.43 (m, 1H), 5.85-5.67 (m, 1H), 5.18 (d, J=6.8, 1H), 4.22 (dd, J=12.0, 6.5, 1H), 3.96 (m, 1H), 3.77 (dd, J=4.7, 3.6, 1H), 3.70, 3.71 (2 s, 6H), 3.44 (s, 3H), 3.38-3.34 (m, 1H), 3.17 (d, J=8.9, 1H).

### Example 21.5

### Preparation of 3-(5′-O-Dimethoxytrityl-2′-O-methyl-β-D-ribofuranosyl)-1,3-diaza-2-oxophenoxazine 3′-O-(2-cyanoethyl) N,N-diisopropylphosphoramidite (21-7)

To a solution of 21-6 (1.2 g, 1.85 mmol) in dry dichloromethane (10 ml) were added dicyanoimidazole (207 mg, 1.75 mmol) followed by 2-cyanoethyl-N,N,N′,N′-tetraisopropylphosphorodiamidite (0.72 ml, 2.3 mmol) under an argon atmosphere and the mixture was stirred at room temperature for 18 h. The reaction mixture was diluted with ethyl acetate (75 ml) and washed with saturated sodium bicarbonate (100 ml). Organic layer was dried over sodium sulfate and evaporated. The residue was purified by silica gel column chromatography and the product was eluted using a mixture of dichloromethane and ethyl acetate containing triethylamine. Appropriate fractions containing the product were pooled and evaporated. The solid obtained was dissolved in dichloromethane (5 ml) and the solution was added to rapidly stirred pentane (300 ml). The precipitated product was collected by filtration and dried to give 1.3 g (82.8%) of 21-7.

³¹P NMR (162 MHz, CD₃CN) δ 155.33, 154.90

### Example 22

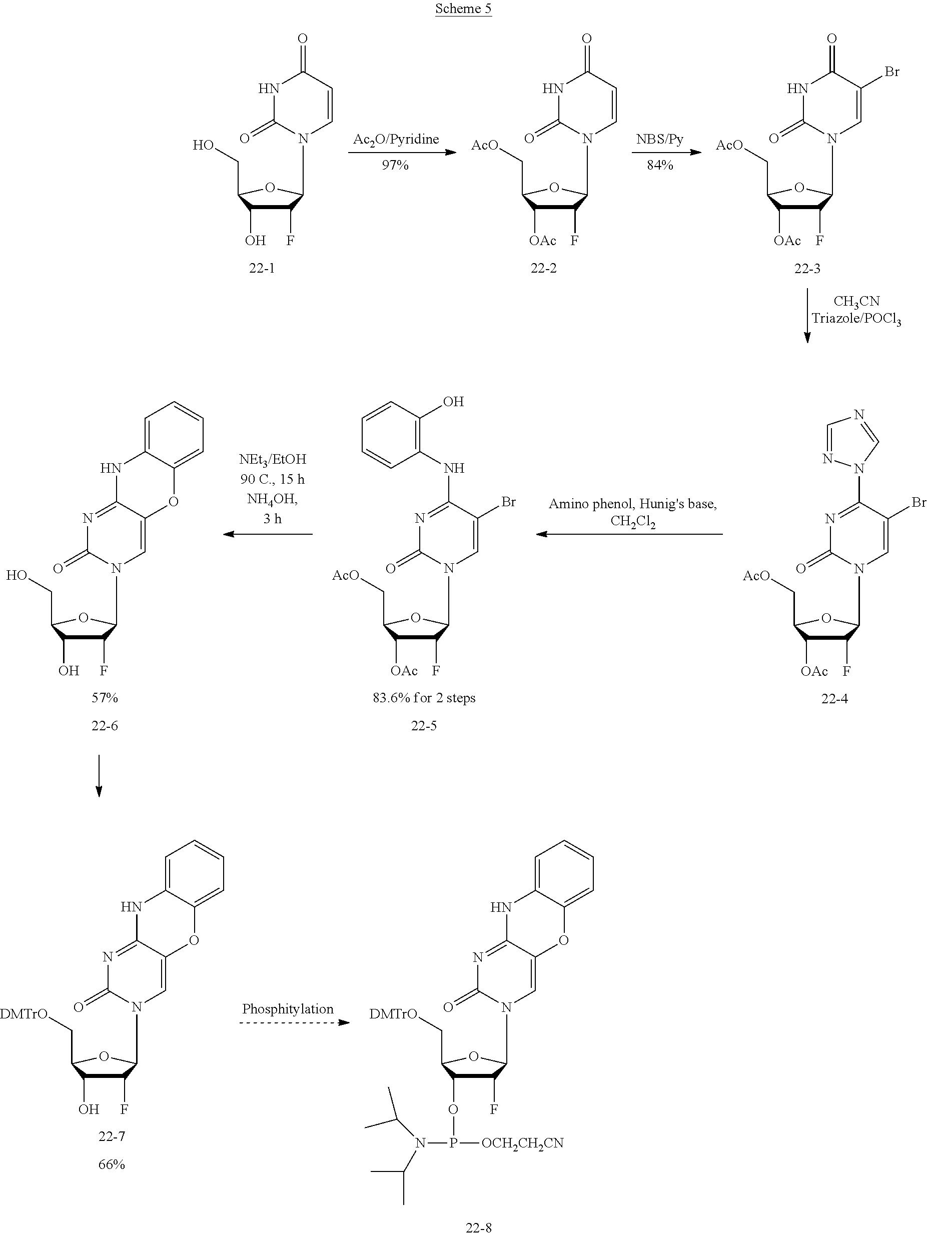

### Example 22.1

### Preparation of 5-Bromo-3′,5′-diacetyl-2′-fluoro-2′-deoxyuridine (22-3)

To a suspension of 2′-fluoro-2′-deoxyuridine (24.6 g, 100 mmol) in dry pyridine (150 ml) was added acetic anhydride (37.78 ml, 400 mmol). After stiffing the mixture at room temperature for 18 h, the reaction was quenched by the addition of methanol (20 ml). After 30 min the solvent was evaporated. To the residue dichloromethane (100 ml) was added and the colorless solid was collected by filtration. The solid product was washed with dichloromethane (150 ml) and dried to give 22 g of 2. The dichloromethane solution which contained some product was washed with sat. sodium bicarbonate solution (3×200 ml). Organic layer was dried over sodium sulfate and evaporated. The residue was coevaporated with toluene to give a colorless solid which was further dried to give a total of 32 g (97%) of 22-2.

The diacetyl derivative 22-2 obtained was suspended in dry pyridine (150 ml) to which N-bromosuccinimide (22 g, 123.6 mmol) was added and stirred at room temperature for 18 h. After evaporating the solvent the residue was dissolved in dichloromethane (350 ml) and washed with water (3×200 ml) followed by sat. sodium bicarbonate solution (150 ml). Organic layer was dried over sodium sulfate and evaporated. The residue was coevaporated with toluene and the crude product was purified by silica gel column chromatography using a gradient of 0-1.5% methanol in dichloromethane to give 33.3 g (84%) of 22-3.

### Example 22.2

### Preparation of 5-Bromo-3′,5′-diacetyl-N⁴-(2-hydroxyphenyl)-2′-fluoro-2′-deoxycytidine (22-5)

To a suspension of triazole (50.34 g, 729 mmol) and phosphorus oxychloride (14.55 ml, 156 mmol) in dry acetonitrile (400 ml) was added triethylamine (97.2 ml, 697 mmol) slowly at 0 C. (ice bath). After stiffing for 20 min a solution of diacetyl-5-bromo-2′-fluoro-2′-deoxyuridine 22-3 (33 g, 80.65 mmol) in dry acetonitrile (150 ml) was added to the reaction flask. Ice bath was removed and the mixture was stirred at room temperature for 3 h. Excess triethylamine (70 ml) and water (30 ml) were added and stirred for 30 min. The solvent was evaporated to a residue and partitioned between dichloromethane and sodium bicarbonate solution. Aqueous layer was extracted with dichloromethane and the combined organic layer was dried over sodium sulfate. The solvent was evaporated to give a light yellow solid which was dried under high vacuum overnight to give the triazole derivative in quantitative yield and directly used in the next step without further purification.

To the triazole derivative 22-4 (22 g, 47.8 mmol) in dichloromethane (200 ml) was added aminophenol (13 g, 120 mmol) followed by Hunig's base (20.8 ml, 120 mmol). The reaction mixture was stirred at room temperature overnight, washed with 5% citric acid (4×350 ml). Aqueous layer was extracted with dichloromethane (100 ml) and the combined organic layer was dried over sodium sulfate and evaporated to give a foam. This was purified on a silica gel column using a gradient of 0-2% methanol in dichloromethane. The appropriate fractions containing the pure product were evaporated to give 20 g (83.6%) of 22-5.

¹H NMR (400 MHz, DMSO) δ 10.21 (s, 1H), 8.45 (s, 1H), 8.22 (s, 1H), 8.05 (dd, J=8.0, 1.1, 1H), 7.01 (td, J=8.0, 1.5, 1H), 6.90 (dd, J=8.0, 1.2, 1H), 6.89-6.78 (m, 2H), 5.85 (dd, J=21.9, 1.1, 1H), 5.55-5.38 (m, 1H), 5.27 (s, 1H), 4.39-4.25 (m, 2H), 4.26-4.14 (m, 1H), 2.05, 2.08 (2 s, 6H).

### Example 22.3

### Preparation of 3-(2′-Fluoro-2′-deoxy-β-D-ribofuranosyl)-1,3-diaza-2-oxophenoxazine (22-6)

To a suspension of 3′,5′-diacetyl-4(hydroxyphenyl)-5-bromo-2′-fluoro-2′-deoxycytidine 22-5 (1.2 g, 2.4 mmol) in absolute alcohol (100 ml) was added triethylamine (20 ml) and the reaction flash was placed in an oil bath at 90 C. for 18 h. The reaction mixture was allowed to cool to room temperature. To the cooled solution 100 ml of ammonium hydroxide was added and the mixture was stirred at room temperature for 3 h. The solvent was evaporated and the solid obtained was purified on a silica gel column using a gradient of 0-10% methanol in ethyl acetate. The appropriate fractions containing the pure product were evaporated to give 0.46 g (58%) of 22-6.

¹H NMR (400 MHz, DMSO) δ 10.66 (s, 1H), 7.71 (s, 1H), 6.98-6.72 (m, 4H), 5.84 (dd, J=17.0, 1.2, 1H), 5.58 (d, J=6.3, 1H), 5.42-5.28 (m, 1H), 5.07-4.90 (m, 1H), 4.90 (s, 1H) 3.82 (dt, J=12.2, 11.5, 2H), 3.68-3.51 (m, 1H), 3.34 (s, 1H).

¹⁹F-NMR: −204.68

### Example 22.4

### Preparation of 3-(5′-O-Dimethoxytrityl-2′-fluoro-2′-deoxy-β-D-ribofuranosyl)-1,3-diaza-2-oxophenoxazine (22-7)

To a solution of 2′-fluorophenoxazine 22-6 (0.44 g, 1.3 mmol) in dry pyridine (20 ml) was added DMT-Cl (0.66 g, 1.95 mmol) and the reaction mixture was stirred at room temperature for 3 h. An additional 140 mg of DMT-Cl was added and after stiffing for the 1.5 h, diluted with dichloromethane (100 ml) and washed with water (50 ml). Organic layer was dried over sodium sulfate and evaporated. The residue was purified by silica gel column chromatography using a gradient of 0-5% methanol in dichloromethane. The appropriate fractions containing the product were evaporated to give 0.55 g (66%) of 22-7.

¹H NMR (400 MHz, DMSO) δ 10.66 (s, 1H), 7.49-7.16 (m, 10H), 6.84 (dddd, J=18.1, 9.2, 6.8, 3.1, 7H), 6.43 (dd, J=11.3, 10.1, 1H), 5.80 (d, J=18.9, 1H), 5.64 (d, J=6.9, 1H), 5.00 (dd, J=53.2, 4.3, 1H), 4.46-4.26 (m, 1H), 4.02 (dd, J=14.1, 7.0, 1H), 3.68, 3.70 (2 s, 6H), 3.41-3.20 (m, 2H).

### Example 22.5

### Preparation of 3-(5′-O-Dimethoxytrityl-2′-fluoro-2′-deoxy-β-D-ribofuranosyl)-1,3-diaza-2-oxophenoxazine 3′-O-(2-cyanoethyl) N,N-diisopropylphosphoramidite (22-8)

Compound 22-7 (1 mmol) on treatment with 2-cyanoethyl-N,N,N′,N″-tetraisopropylphosphorodiamidite (1.2 mmol) in dichloromethane in the presence of 3,4-dicyanoimidazole (0.9 mmol), work up of the reaction followed by silica gel column chromatography gives the corresponding 3′-phposphoramidite 22-8. Alternatively this compound can also be prepared by treating 22-7 with 2-cyanoethyl-N,N-diisopropylchloro phosphoramidite in dichloromethane in the presence of Hunig's base.

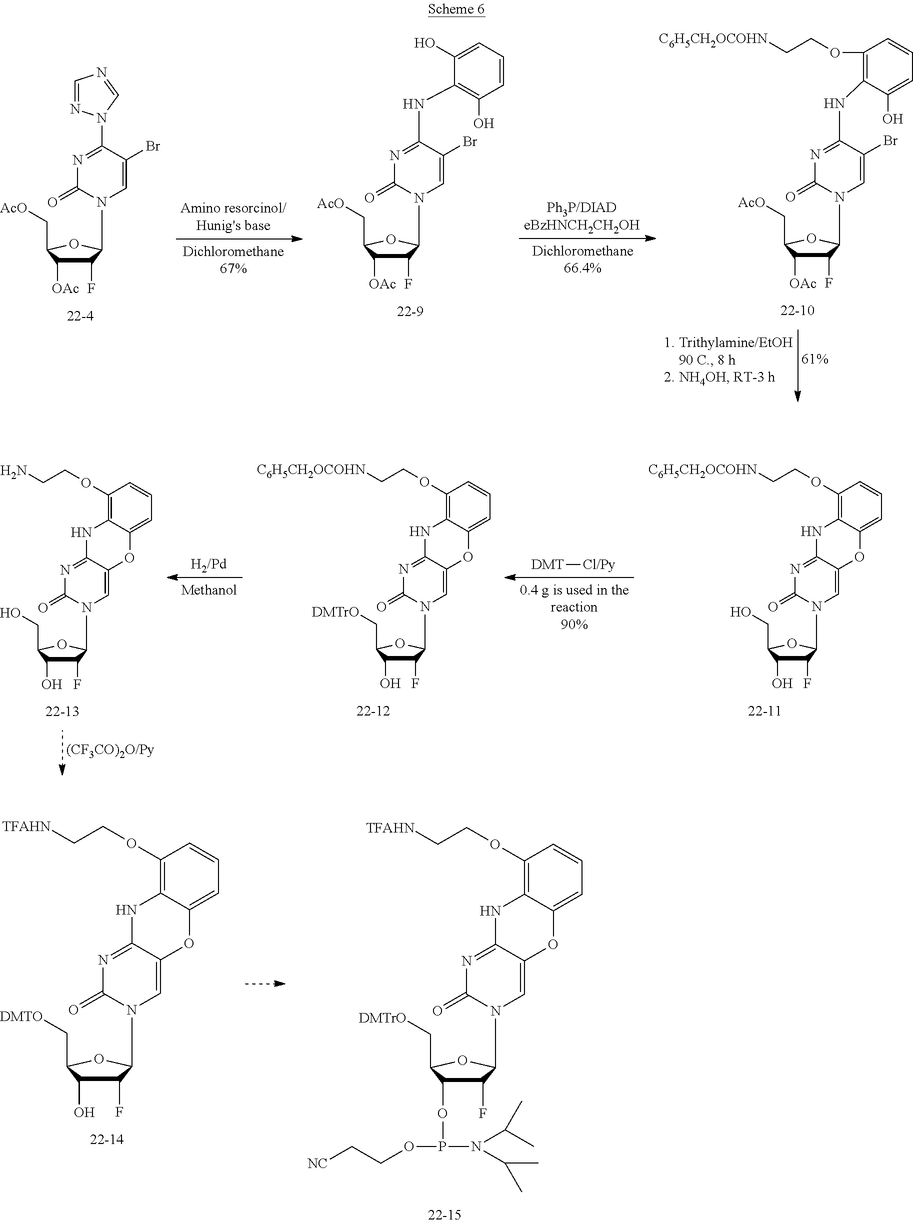

### Example 22.6

### Preparation of 5-Bromo-3′,5′-diacetyl-N⁴-(2,6-dihydroxyphenyl)-2′-fluoro-2′-deoxycytidine (22-9)

To a solution of triazolyl derivative 22-4 (9.2 g, 20 mmol) in dry dichloromethane (175 ml) was added amino resorcinol hydrochloride (4.5 g, 27.9 mmol) followed by Hunig's base (9 ml, 52 mmol). After stirring the mixture at room temperature for 18 h, diluted with dichloromethane (200 ml) and washed with 5% citric acid (2×250 ml). Organic layer was dried over sodium sulfate and evaporated. The crude product was purified by silica gel column chromatography using a gradient of 0-5% methanol in dichloromethane. The appropriate fractions containing the product were evaporated to give 6.88 g (66.7%) of 22-9.

### Example 22.7

### Preparation of 5-Bromo-3′,5′-diacetyl-N⁴-(2-(2-N-benzylcarbamyl-ethoxy)-6-hydroxyphenyl)-2′-fluoro-2′-deoxycytidine (22-10)

To a solution of diacetyl-4-(dihydroxyphenyl)-5-bromo-2′-fluoro-2′-deoxycytidine (6.5 g, 12.6 mmol) in dry dichloromethane (175 ml) was added triphenylphosphine (4.98 g, 19 mmol) and benzyl-N-(2-hydroxyethyl)carbamate (3.12 g, 16 mmol). The reaction flask was cooled in an ice bath and diisopropylazodicarboxylate (3.68 ml, 19 mmol) was added in about 5 min. The reaction mixture was stirred overnight by allowing it to slowly warm to room temperature. The reaction mixture was diluted with dichloromethane and washed with water. Organic layer was dried and evaporated. The product was purified by silica gel column chromatography using a gradient of 0-5% methanol in dichloromethane. The appropriate fractions containing the product were evaporated to give 5.8 g (66.4%) of 22-10.

¹H NMR (400 MHz, DMSO) δ 9.67 (s, 1H), 8.36 (s, 1H), 8.16 (s, 1H), 7.51-7.22 (m, 7H), 7.06 (t, J=8.3, 1H), 5.84 (d, J=21.1, 1H), 5.42 (dd, 1H), 5.22-5.32 (m, 1H), 4.99 (s, 2H), 4.43-4.15 (m, 3H), 3.98 (t, J=5.6, 2H), 3.44-3.22 (m, 2H), 2.05, 2.08 (2 s, 6H). MS: Calcd: 692.11, 694.11. Found: 691 (M−H), 693 (M−H).

### Example 22.8

### Preparation of 3-(2′-Fluoro-2′-deoxy-β-D-ribofuranosyl)-9-(2-N-benzylcarbamyl-ethoxy)-1,3-diaza-2-oxophenoxazine (22-11)

A solution of 22-10 (4.5 g, 6.49 mmol) in a mixture of absolute alcohol (400 ml) and triethylamine (80 ml) was heated in an oil bath at 90 C. for 15. The mixture was allowed to cool to room temperature and ammonium hydroxide (200 ml) was added to the reaction flask. After stirring for 3 h, it was evaporated to dryness. The residue was coevaporated with ethanol followed by dichloromethane and purified by silica gel column chromatography using a gradient of 0-8% methanol in ethyl acetate to yield 2.1 g (61%) of pure product.

¹H NMR (400 MHz, DMSO) δ 9.88 (s, 1H), 7.78 (d, J=11.5, 2H), 7.47-7.23 (m, 4H), 6.80 (t, J=8.3, 1H), 6.61 (d, J=8.3, 1H), 6.44 (d, J=8.2, 1H), 5.86 (d, J=17.0, 1H), 5.55 (s, 1H), 5.33 (s, 1H), 5.04 (s, 2H), 4.90 (dd, 1H), 4.14 (d, J=22.3, 1H), 3.92 (s, 2H), 3.86 (d, J=7.8, 1H), 3.78 (d, J=12.0, 1H), 3.60 (d, J=12.1, 1H), 3.41 (d, J=4.4, 2H). ¹⁹F-NMR: m, −204.51-−204.76

### Example 22.9

### Preparation of 3-(5′-O-Dimethoxytrityl-2′-fluoro-2′-deoxy-β-D-ribofuranosyl)-9-(2-N-benzylcarbamyl-ethoxy)-1,3-diaza-2-oxophenoxazine (22-12)

To a solution of 22-11 (0.4 g, 0.75 mmol) in dry pyridine (10 ml) was added DMT-Cl (338 mg, 1 mmol) and the mixture was stirred at room temperature for 4 h. The reaction mixture was diluted with dichloromethane (100 ml) and washed with water (50 ml). Organic layer was dried over sodium sulfate and evaporated. The residue was coevaporated with toluene and purified by silica gel column chromatography using a gradient of 0-5% methanol in dichloromethane to give 0.57 g (90.7%) of pure product.

### Example 22.10

### Preparation of 3-(5′-O-Dimethoxytrityl-2′-fluoro-2′-deoxy-β-D-ribofuranosyl)-9-(2-N-trifluoroacetamido-ethoxy)-1,3-diaza-2-oxophenoxazine (22-13)

To a solution of 22-12 (0.4 g, 0.48 mmol) in a mixture of methanol (25 ml) and triethylamine (2 ml) was added 10% palladium on carbon (75 mg, Degussa type) and the mixture was stirred under a positive pressure of H₂ for 4 h. The reaction mixture was filtered and evaporated. The residue was coevaporated with dry pyridine. The residue was dissolved in pyridine (6 ml) and the reaction flask was cooled in an ice bath. To this cold solution trifluoroacetic anhydride (0.6 ml) was added dropwise. After stiffing the mixture for 3 h, diluted with dichloromethane and washed with water. Organic layer was dried over sodium sulfate and evaporated. The residue was coevaporated with toluene and purified by silica gel column chromatography using a gradient of 0-4% methanol in dichloromethane to give 0.21 g (55%) of pure 22-13.

¹H NMR (400 MHz, DMSO) δ 9.88 (s, 1H), 9.73 (s, 1H), 7.55-7.17 (m, 10H), 6.88 (dd, J=8.8, 4.8, 4H), 6.78 (t, J=8.3, 1H), 6.59 (d, J=8.3, 1H), 6.10 (d, J=8.1, 1H), 5.81 (d, J=18.9, 1H), 5.66 (d, J=6.9, 1H), 5.12-4.88 (m, 1H), 4.47-4.29 (m, 1H), 4.00 (dd, J=9.6, 5.9, 2H), 3.68, 3.70 (2 s, 6H), 3.63 (d, J=4.4, 2H), 3.47-3.29 (m, 1H), 3.25 (d, J=10.2, 1H). ¹⁹F-NMR: s, −77.1; m, −203.1.

### Example 22.11

### Preparation of 3-(5′-O-Dimethoxytrityl-2′-fluoro-2′-deoxy-β-D-ribofuranosyl)-9-(2-N-trifluoroacetamido-ethoxy)-1,3-diaza-2-oxophenoxazine 3′-O-(2-cyanoethyl) N,N-diisopropylphosphoramidite (22-14)

Compound 22-13 (1 mmol) on treatment with 2-cyanoethyl-N,N,N′,N″-tetraisopropylphosphorodiamidite (1.2 mmol) in dichloromethane in the presence of 3,4-dicyanoimidazole (0.9 mmol), work up of the reaction followed by silica gel column chromatography gives the corresponding 3′-phposphoramidite 22-14. Alternatively this compound can also be prepared by treating with 2-cyanoethyl-N,N-diisopropylchloro phosphoramidite in dichloromethane in the presence of Hunig's base.

### Example 23

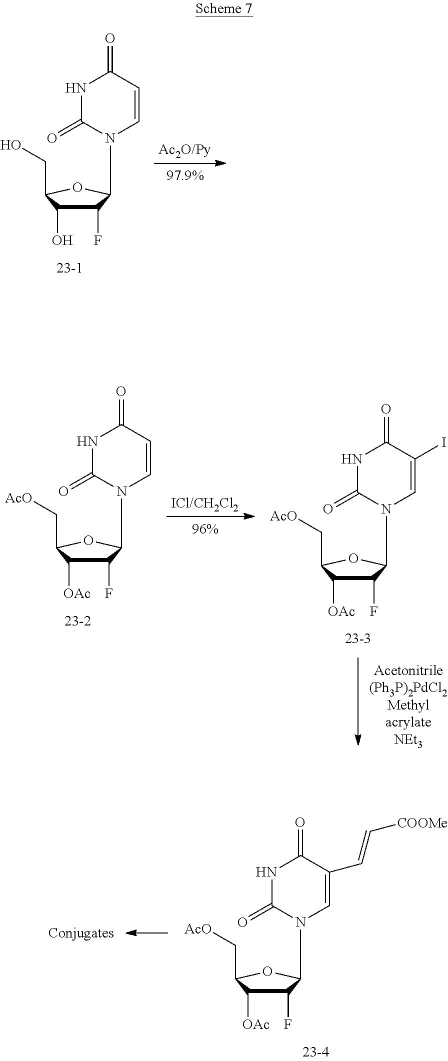

### Example 23.1

### Preparation of 3′,5′-Diacetyl-5-iodo-2′-fluoro-2′-deoxyuridine (23-3, J. shi, J. Du, T. Ma. K. W. Pankiewicz, S. E. Patterson, P. M. Tharnish, T. M. McBrayer, L. J. Stuyver, M. J. Otto, C. K. Chu, R. F. Schinazi, K. a. Watanabe, Bioorg. Med. Chem., 2005, 13, 1641-1652):

To a solution of 2′-fluorouridine (2.46 g, 10 mmol) in anhydrous pyridine (15 ml) was added acetic anhydride (5.67 ml, 60 mmol) and the mixture was stirred at room temperature for 18 h. The reaction was quenched by the addition of methanol (5 ml) and after 30 min at room temperature the reaction mixture was diluted with dichloromethane (150 ml) and washed with sat. sodium bicarbonate solution (250 ml). Organic layer was dried over sodium sulfate and evaporated. The residue was coevaporated with toluene and dried on a high vacuum pump to give 3.23 g (97.9%) of 23-2 as a colorless foam.

To a solution of 23-2 (1.65 g, 5 mmol) in dichloromethane (50 ml) was added iodine monochloride (1.5 g, 9.24 mmol) and the mixture was heated under reflux for 5 h. The reaction mixture was allowed to cool to room temperature and washed with 5% sodium bisulfite solution (100 ml) followed by sat. sodium bicarbonate solution (200 ml). Organic layer was dried and evaporated to give 2.2 g (96%) of 23-3.

¹H NMR (400 MHz, DMSO) δ 11.84 (s, 1H), 8.18 (s, 1H), 5.85 (dd, J=22.1, 1.5, 1H), 5.52 (ddd, J=52.5, 5.2, 1.7, 1H), 5.34-5.18 (m, 1H), 4.41-4.24 (m, 2H), 4.18 (dd, J=12.1, 5.2, 1H), 2.07, 2.10 (2 s, 6H).

¹⁹F-NMR: m, −200.75.

### Example 23.2

### Preparation of 3′,5′-Diacetyl-5-[2-(Methoxycarbonyl)ethenyl]-2′-fluoro-2′-deoxyuridine (23-4)

To a solution of 23-3 (1.82 g, 4 mmol) in dry acetonitrile (50 ml) was added triethylamine (1.2 ml), methyl acrylate (0.54 ml, 6 mmol) and dichlorobis(triphenylphosphine)palladium(II) (35 mg) and the mixture was heated at 90° C. for 18 h. The solvent was evaporated and the residue was purified by silica gel column chromatography to give 1.5 g (90.5%) of 23-4.

¹H NMR (400 MHz, DMSO) δ 11.83 (s, 1H), 8.24 (s, 1H), 7.37 (d, J=15.8, 1H), 6.87 (d, J=15.9, 1H), 5.90 (d, J=20, 1H), 5.53 (ddd, J=52.3, 5.4, 1.5, 1H), 5.34-5.18 (m, 1H), 4.42-4.20 (m, 3H), 3.67 (s, 3H), 2.11 (s, 3H), 2.03 (s, 3H).

¹⁹F-NMR: m, −200.8.

### Example 24

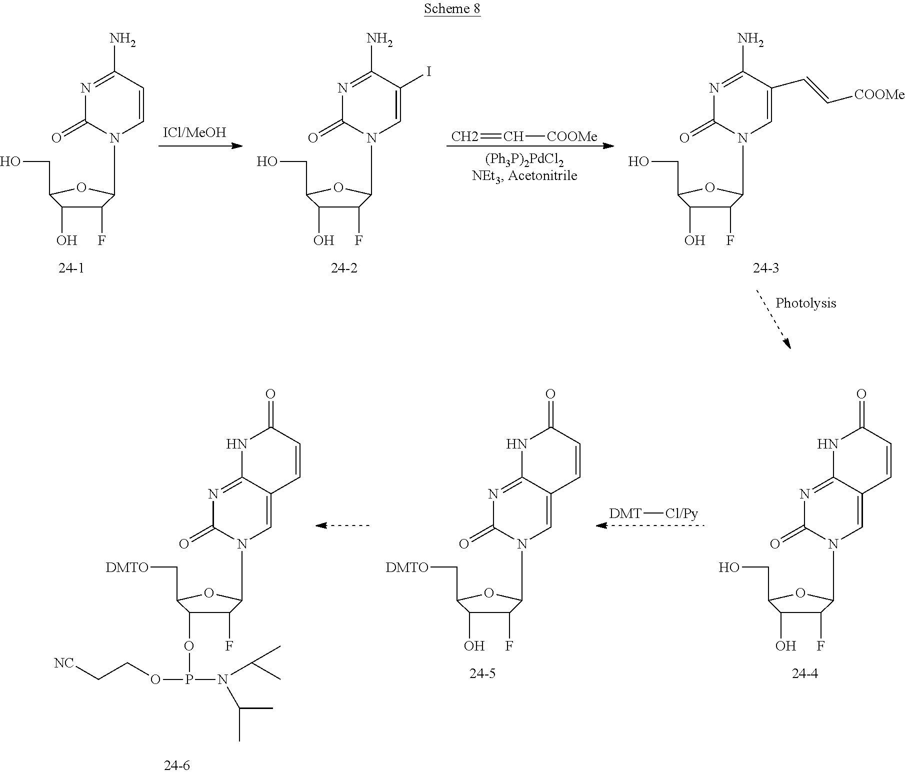

### Example 24.1

### Preparation of 5-Iodo-2′-fluoro-2-deoxycytidine (24-2, J. shi, J. Du, T. Ma. K. W. Pankiewicz, S. E. Patterson, P. M. Tharnish, T. M. McBrayer, L. J. Stuyver, M. J. Otto, C. K. Chu, R. F. Schinazi, K. a. Watanabe, Bioorg. Med. Chem., 2005, 13, 1641-1652):

To a suspension of 2′-Fluoro-2′-deoxycytidine (3.8 g, 15.45 mmol) in dry methanol (150 ml) was added iodine monochloride (5 g, 30.8 mmol) and the mixture was heated at 50° C. for 7 h. The reaction mixture was allowed to cool to room temperature and the solvent was evaporated. The residue was triturated with dichloromethane to remove excess reagent and the crude product was purified by silica gel column chromatography.

### Example 24.2

### Preparation of 5-[2-(Methoxycarbonyl)ethenyl]2′-Fluoro-2′-deoxycytidine (24-3)

To a solution of 24-2 (0.75 g, 2 mmol) in a mixture of dry acetonitrile (6 ml) and DMF (24 ml) was added triethylamine (0.6 ml), methyl acrylate (0.27 ml, 3 mmol) and dichlorobis(triphenylphosphine)palladium(II) (50 mg) and the mixture was heated at 90° C. for 26 h. The solvent was evaporated and the residue was purified by silica gel column chromatography to give 24-4.

### Example 24.3

### Preparation of 3-(2-Fluoro-2-deoxy-β-D-erythro-pentofuranosyl)pyrido[2,3-d]pyrimidine-2,7(8H)-dione (24-4)

Irradiation of a solution of 24-3 in water at 300 nm provides compound 24-4. This can be isolated by evaporation of the solvent and crystallization or by column chromatography.

### Example 24.4

### Preparation of 3-[5-O-(4,4′-Dimethoxytrityl)-(2-fluoro-2-deoxy-β-D-erythro-pentofuranosyl)pyrido[2,3-d]pyrimidine-2,7(8H)-dione 3′-O-(2-cyanoethyl) N,N-diisopropylphosphoramidite (24-6)

3-(2-Fluoro-2-deoxy-β-D-erythro-pentofuranosyl)pyrido[2,3-d]pyrimidine-2,7(8H)-dione 24-4 (μmol) on reaction with 4,4′-dimethoxytrityl chloride (1.25 mmol) in dry pyridine provides the dimethoxytrityl derivative 24-5 which on phosphitylation with 2-cyanoethyl-N,N,N′,N″-tetraisopropylphosphorodiamidite (1.2 eq.) in dichloromethane in the presence of 3,4-dicyanoimidazole (0.9 eq.), work up of the reaction followed by silica gel column chromatography gives the corresponding 3′-phposphoramidite 24-6. Alternatively this compound can also be prepared by treating 24-5 with 2-cyanoethyl-N,N-diisopropylchloro phosphoramidite in dichloromethane in the presence of Hunig's base.

### REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

### Compounds I-35

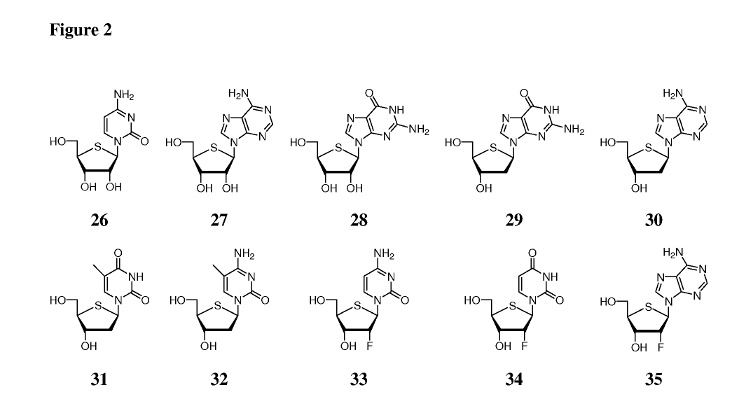

The references below in Table 5 are correlated with compounds described herein (see, for example, FIGS. 1 and 2).

TABLE 5
References Related to Compounds 1-35.

| Compound(s) | Reference |
|---|---|
| 1 | Cook et al. Synthesis of 2′-O-substituted pyrimidine nucleosides via |
| | nucleophilic alkylation in preparation of substituted oligoribonucleotide |
| | duplexes. U.S. Pat. No. 5,760,202 |
| | Codington et al. Nucleosides. XVIII. Synthesis of 2′-fluorothymidine, |
| | 2′fluorodeoxyuridine, and other 2′-halo-2′-deoxy nucleosides. J. Org. |
| | Chem. 1964, 29, 558-64. |
| 2 | An et al. Synthesis of novel 3′-C-methylene thymidine and 5- |
| | methyluridine/cytidine H-phosphonates and phosphonamidites for new |
| | backbone modification of oligonucleotides. J. Org. Chem. 2001, 66, 2789. |
| | Baker and Cowsert, Antisense oligonucleotide inhibition of human TNFR1 |
| | expression and its therapeutic uses U.S. Pat. No. 6,007,995. |
| 3 | Shi et al. Synthesis and in vitro anti-HCV activity of β-D- and L-2′-deoxy-2′- |
| | fluororibonucleosides. Nucleosides, Nucleotides & Nucleic Acids 2005, 24, |
| | 875. |
| | Miraglia et al. Antisense modulation of mdm2 expression. US publication |
| | 2003203862. |
| 4 | Ross et al. Kilo-scale synthesis process for 2′-O-(2-methoxyethyl)-pyrimidine |
| | derivatives. Nucleosides, nucleotides & Nucleic acids, 2005, 24, 815. |
| | Bhat et al. Modulation of human survivin expression by compounds |
| | comprising single and double-stranded oligonucleotides and uses for cancer |
| | therapy. WO 2005/002507. |
| 5 | Murakami et al. The mechanism of action of β-D-2′-deoxy-2′-fluoro-2′-C- |
| | methylcytidine involves a second metabolic pathway leading to β-D-2′- |
| | deoxy-2′-fluoro-2′-C-methyluridine 5′-triphosphate, a potent inhibitor of the |
| | hepatitis C virus RNA-dependent RNA polymerase. Antimicrobial |
| | Agents and Chemotherapy 2008, 52, 458. |
| | Clark et al. Design, Synthesis, and Antiviral Activity of 2′-Deoxy-2′-fluoro- |
| | 2′-C-methyl-cytidine, a Potent Inhibitor of Hepatitis C Virus Replication. J. |
| | Org. Chem. 2005, 48, 5504. |
| 6 | Benzaria et al. 2′-C-methyl branched pyrimidine ribonucleoside analogues: |
| | potent inhibitors of RNA virus replication. Antiviral Chemistry & |
| | Chemotherapy 2007, 18, 225. |
| | Roberts et al. Preparation of nucleoside derivatives for treating hepatitis C |
| | virus infection WO 2003/093290. |
| 7 | Johansen et al. Compositions and methods for treatment of viral diseases. |
| | WO 2008/033466. |
| | Clark et al. Design, Synthesis, and Antiviral Activity of 2′-Deoxy-2′-fluoro- |
| | 2′-C-methyl-cytidine, a Potent Inhibitor of Hepatitis C Virus Replication. J. |
| | Org. Chem. 2005, 48, 5504. |
| 8 | Hong et al. Preparation of sugar modified nucleosides as antiviral agents. |
| | WO 2003/062255. |
| 9 | Damhan and Peng 2′-Deoxy-2′-fluoro-β-D-arabinonucleoside 5′- |
| | triphosphates and their use in enzymic nucleic acid synthesis. WO |
| | 2008/022469. |
| 10 | Watanabe, K. Role of modified nucleosides in the translation function of |
| | tRNAs from extreme thermophilic bacteria and animal mitochondria. Bull. |
| | Chem. Soc. Japan 2007, 80, 1253. |
| | Baker et al. SiRNA having modified bases for binding to adenine and |
| | guanine and their use in gene modulation and disease treatment. WO |
| | 2004/044245. |
| 11 | Diop-Frimpong et al. Stabilizing contributions of sulfur-modified |
| | nucleotides: crystal structure of a DNA duplex with 2′-O-[2- |
| | (methoxy)ethyl]-2-thiothymidines. Nucleic Acids Res. 2005, 33, 5297. |
| | Rajeev et al. 2′-Modified-2-thiothymidine Oligonucleotides. Organic Lett. |
| | 2003, 5, 3005. |
| 12 | Hardee and Dellamary Compositions and methods for the treatment of |
| | severe acute respiratory syndrome (SARS). WO 2005/020885. |
| 13 | Sochacka et al. Nucleosides. 153. Synthesis of 1-methyl-5-(3-azido-2,3- |
| | dideoxy-β-D-erythro-pentofuranosyl)uracil and 1-methyl-5-(3-azido-2,3- |
| | dideoxy-2-fluoro-β-D-arabinofuranosyl)uracil. The C-nucleoside isostere |
| | of 3′-azido-3′-deoxythymidine and its 2′-“up”-fluoro analog. J. Med. Chem. |
| | 1990, 33, 1995. |
| 14 | Mokany et al. Multicomponent nucleic acid enzymes and methods for their |
| | use. WO 2007/041774. |
| | Ross et al. Synthesis and incorporation of 2′-O-methyl-pseudouridine into |
| | oligonucleotides. Nucleosides & Nucleotides, 1997), 16, 1547. |
| 15 | Wigler et al. Effects of 4-thiopseudouridine on the salvage of pseudouridine |
| | by Escherichia coli cells. J. Carbohydrates, Nucleosides, Nucleotides |
| | 1974, 1, 307. |
| 16 | Xia et al. Gene Silencing Activity of siRNAs with a Ribo-difluorotoluyl |
| | Nucleotide. ACS Chem. Biol. 2006, 1, 176. |
| | Manoharan et al. Single- and double-stranded oligonucleotides containing |
| | unnatural nucleobases for inhibition of gene expression and treatment of |
| | disease. US patent application publication 2006/035254. |
| 17 | Tuttle et al. Purine 2′-deoxy-2′-fluororibosides as antiinfluenza virus agents. |
| | J. Med. Chem. 1993, 36, 119. |
| | Tisdale et al. Preparation of 2′-deoxy-2′-fluororibonucleosides as medicinal |
| | virucides. EP 417999. |
| | Jin and Kim Pyrimidyl phosphonate antiviral compounds and methods of |
| | use. WO 2005/070901. |
| 18 | Ecker et al. Methods for identification of coronaviruses by PCR |
| | amplification of a RdRp orf-1b or nsp11 target region for diagnostic |
| | application. WO 2004/111187. |
| | Lin and Matteucci Preparation of phenoxazin-1-yl |
| | oligodeoxyribonucleotides as enzyme and gene expression inhibitors. WO |
| | 99/24452. |
| 19 | Holmes et al. Steric inhibition of human immunodeficiency virus type-1 Tat- |
| | dependent trans-activation in vitro and in cells by oligonucleotides |
| | containing 2′-O-methyl G-clamp ribonucleoside analogues. Ncleic Acids |
| | Research 2003, 31, 2759. |
| 20 | Lin et al., Tricyclic 2′-Deoxycytidine Analogs: Syntheses and Incorporation |
| | into Oligodeoxynucleotides Which Have Enhanced Binding to |
| | Complementary RNA. J. Am. Chem. Soc., 1995, 117, 3873. |
| | Sandin et al., Synthesis and oligonucleotide incorporation of fluorescent |
| | cytosine analogue tC: a promising nucleic acid probe. Nature Protocols, |
| | 2007, 2, 615. |
| 21 | Lin et al., Tricyclic 2′-Deoxycytidine Analogs: Syntheses and Incorporation |
| | into Oligodeoxynucleotides Which Have Enhanced Binding to |
| | Complementary RNA. J. Am. Chem. Soc., 1995, 117, 3873. |
| 22 | Alekseeva et al., Nucleosides with tricyclic aglycone. The ribonucleosides of |
| | condensed 1,2,4-triazine: synthesis and antiherpetic activity. Biopolimeri i |
| | Klitina, 2006, 22, 468. |
| 23 | Leydier et al. 4′-Thio-β-D-oligoribonucleotides: nuclease resistance and |
| | hydrogen bonding properties. Nucleosides & Nucleotides 1995, 14, 1027. |
| 24-25 | Bhat et al. Preparation of antisense 4′-thionucleosides-containing |
| | oligoribonucleotides duplexes for use in the RNA interference pathway of |
| | gene modulation. WO 2005/027962. |
| 26 | Yoshimura et al. A practical synthesis of 4′-thioribonucleosides. Tetrahedron |
| | Letters, 2006, 47, 591. |
| | Hoshika et al. Synthesis and physical and physiological properties of 4′-thio- |
| | RNA: application to post-modification of RNA aptamer toward NF-κ B. |
| | Nucleic Acids Res. 2004, 32, 3815. |
| 27 | Dande et al. Improving RNA Interference in Mammalian Cells by 4′-Thio- |
| | Modified Small Interfering RNA (siRNA): Effect on siRNA Activity and |
| | Nuclease Stability When Used in Combination with 2′-O-Alkyl |
| | Modifications. J. Med. Chem. 2006, 49, 1624 |
| 28 | Hoshika et al. Synthesis and physical and physiological properties of 4′-thio- |
| | RNA: application to post-modification of RNA aptamer toward NF-κ B. |
| | Nucleic Acids Res. 2004, 32, 3815. |
| | Matsuda et al. Preparation of 4′-thionucleotide and oligonucleotides |
| | containing them. WO 2004/018494. |
| 29-32 | Inoue et al. Practical Synthesis of 2′-Deoxy-4′-thioribonucleosides: |
| | Substrates for the Synthesis of 4′-ThioDNA. J. Org. Chem. 2005, 70, 8597. |
| 33-35 | Takahashi et al. Synthesis and crystal structure of 2′-deoxy-2′-fluoro-4′- |
| | thioribonucleosides: substrates for the synthesis of novel modified RNAs. |
| | Tetrahedron, 2008, 64, 4313. |

### EQUIVALENTS

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

We claim:

1. A nucleoside having the formula: 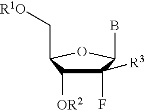
wherein:
R¹ represents —H, —P(O)(OM)₂, —P(O)(OM)-O—P(O)(OM)₂, —P(O)(Oalkyl)₂, —P(O)(Oalkyl)-O—P(O)(Oalkyl)₂, —PO₃H₂, —PO₃HM, —PO₃M₂, —PO₂SH₂, —PO₂SHM, —PO₂SM₂, —PO₃M, or —PO₂SM, a protecting group, a ligand, or a ligand carrying monomer;
R² represents R¹, —F, —(C₁-C₆)alkyl, —(C₂-C₆)allyl, —(C(R₃)₂)ₙOR³, —(C(R³)₂)ₙSR³, —(C(R³)₂)ₙN(R³)₂, —(C(R³)₂)ₙC(O)N(R³)₂, —(C(R³)₂)ₙO(C₁-C₆)alkyl, —(C(R³)₂)ₙS(C₁-C₆)alkyl, —(C(R³)₂)ₙO(C(R³)₂)ₙN((C₁-C₆)alkyl)₂, —(C(R³)₂)ₙON((C₁-C₆)alkyl)₂, —C(O)R³, —C(O)R³C(O)H, —C(O)R³C(O)OH, —C(O)R³C(O)R³, —C(O)R³C(O)NR³—PO₂, —P(OR³)₂, —P(N(R³)₂)₂, or —P(OR³)N(R³)₂;
R³ represents independently for each occurrence hydrogen or a substituted or unsubstituted C₁-C₄ alkyl;
M represents independently for each occurrence an alkali metal or a transition metal with an overall charge of +1;
n is an integer from 1-4; and
B is a nucleobase selected from the group consisting of:

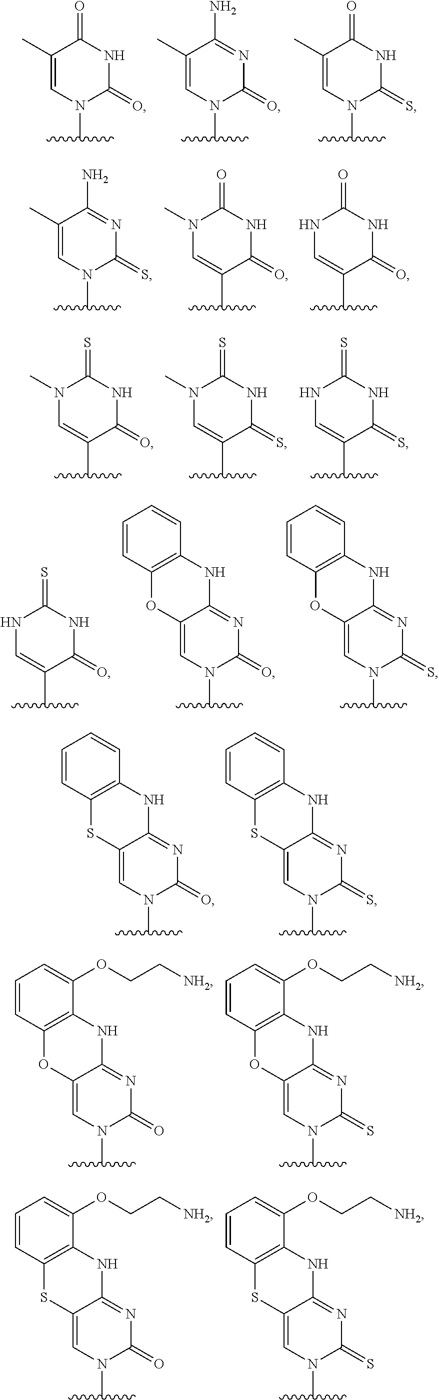

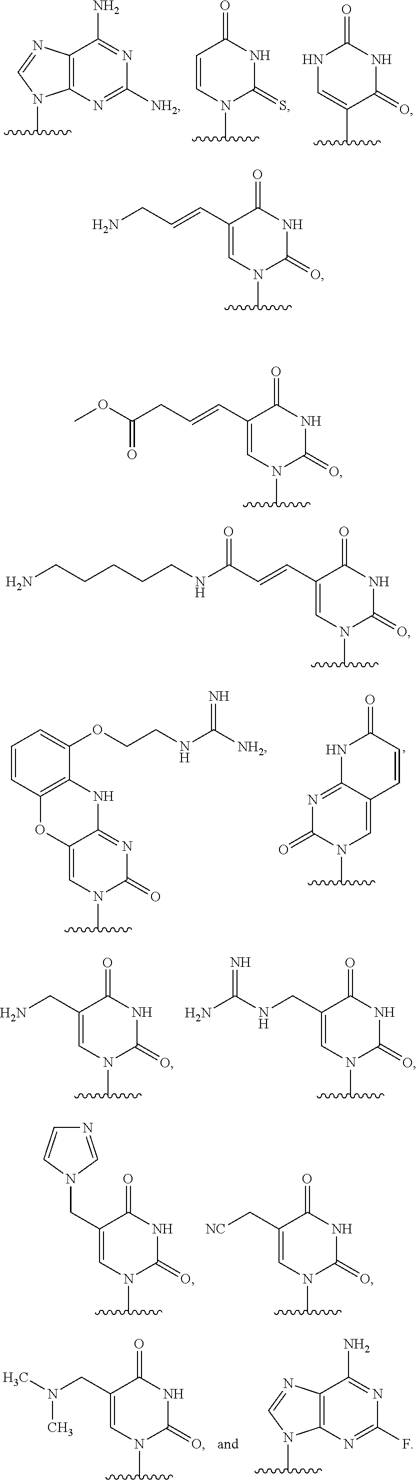

2. The nucleoside of claim 1, wherein said nucleobase is

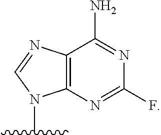

3. The nucleoside of claim 1, wherein said nucelobase is selected from the group consisting of:

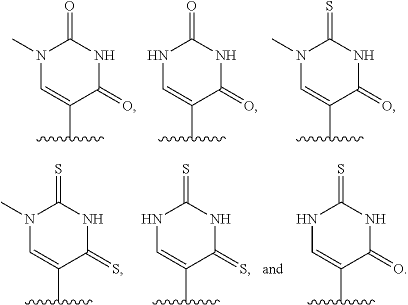

4. The nucleoside of claim 1, wherein said nucelobase is selected from the group consisting of:

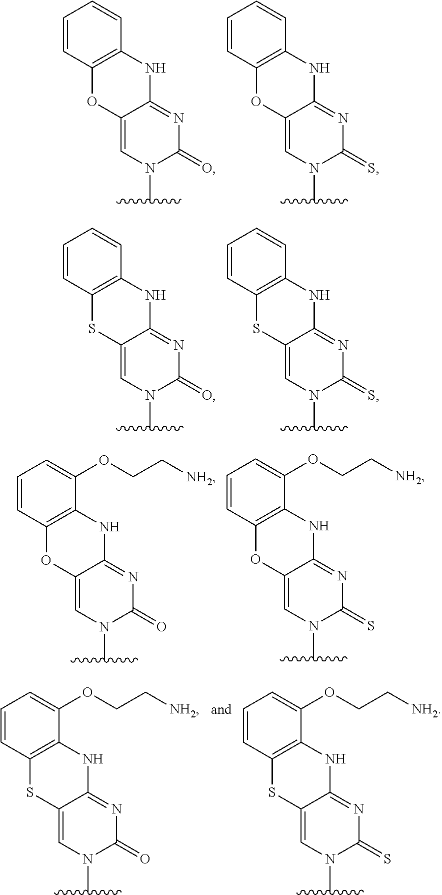

5. The nucleoside of claim 1, wherein R³ is an alkyl group and the nucelobase is selected from the group consisting of:

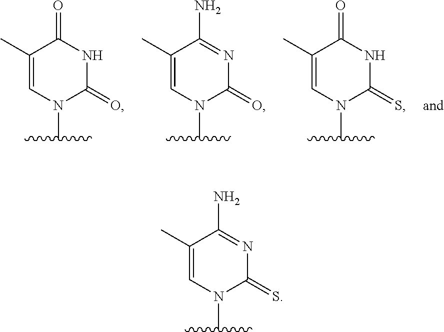

6. A oligonucleotide comprising a nucleoside of claim 1.

7. The oligonucleotide of claim 6, wherein said oligonucleotide comprises at least one non-phosphodiester backbone linkage.

8. The oligonucleotide of claim 7, wherein said non-phosphodiester backbone linkage is selected from a group consisting of phosphorothioate, phosphorodithioate, alkyl-phosphonate and phosphoramidate backbone linkage.

9. The oligonucleotide of claim 6, wherein said nucleoside comprises at least one ligand conjugate.

10. An RNAi agent comprising at least one nucleoside of claim 1.

11. The RNAi agent of claim 10, wherein said RNAi agent is double stranded.

12. The RNAi agent of claim 11, wherein only one strand comprises the nucleoside of claim 1.

13. The RNAi agent of claim 12, wherein the sense strand comprises the nucleoside of claim 1.

14. The RNAi agent of claim 12, wherein the antisense strand comprises the nucleoside of claim 1.

15. The RNAi agent of claim 11, wherein each strand comprise at least one nucleoside of claim 1.

16. The RNAi agent of claim 15, wherein said nucleoside is the same in the two strands.

17. The RNAi agent of claim 15, wherein said nucleoside is different in the two strands.

18. The RNAi agent of claim 10, wherein said RNAi agent is single stranded.

19. The RNAi agent of claim 10, wherein said RNAi agent has a hairpin structure.

20. The RNAi agent of claim 6, wherein said oligonucleotide is an RNAi agent, an antisense, an antagomir, a microRNA, a pre-microRNA, an antimir, a ribozyme or an aptamer.

21. A method of inhibiting the expression of a gene in a cell, the method comprising
a. contacting an RNAi agent of claim 10 with the cell; and
b. maintaining the cell from step (a) for a time sufficient to obtain degradation of the mRNA of the target gene.
